(19) United States
(12) Patent Application Publication
(10) Pub. No.: US 2026/0356891 A1
(43) Pub. Date: Nov. 12, 2026

(54) QUANTUM-INSPIRED LOGIC FOR ADVANCED TRANSCRIPTIONAL (BIOLOGICAL) PROGRAMMING

(71) Applicant: GEORGIA TECH RESEARCH CORPORATION, Atlanta, GA (US)

(72) Inventors: Corey J. WILSON, Atlanta, GA (US); Prasaad T. MILNER, Atlanta, GA (US); Dowan KIM, Atlanta, GA (US)

(21) Appl. No.: 19/659,166

(22) Filed: Apr. 27, 2026

Related U.S. Application Data
(60) Provisional application No. 63/794,758, filed on Apr. 25, 2025.

(51) Int. Cl. C07K 14/00

(52) U.S. Cl. CPC C07K 14/001

(57) ABSTRACT

The present disclosure relates to gene circuits capable of synthetic biological program operations, bi-directional promoters, and cells comprising the disclosed gene circuits and bi-directional promoters.

### CROSS-REFERENCE TO RELATED APPLICATIONS

This application claims the benefit of U.S. Provisional Application No. 63/794,758, filed on Apr. 25, 2025, which is incorporated herein by reference in its entirety.

### GOVERNMENT SUPPORT CLAUSE

This invention was made with government support under Grant No. 2319231, awarded by the National Science Foundation, and Grant No. R35GM153457, awarded by the National Institute of Health. The government has certain rights in the invention.

### REFERENCE TO SEQUENCE LISTING

The sequence listing submitted on Apr. 27, 2026, as an .XML file entitled, 10034-433US1_ST26.xml created on Apr. 21, 2026, and having a file size of 104,494 bytes is hereby incorporated by reference pursuant to 37 C.F.R. § 1.52(e)(5).

### FIELD

The present disclosure relates to gene circuits comprising quantum logic gates and bi-direction promoters capable of synthetic biological program operations.

### BACKGROUND

Synthetic biologists have made significant strides in engineering intelligent biological systems using gene regulatory networks. To date, synthetic biotic systems have drawn inspiration from classical computing, where sets of fundamental logical operations are engineered in single cells and modularly combined to create genetic circuits. These programs have shown great promise in biomanufacturing, metabolic engineering, biocontainment, agriculture, and therapeutic applications, among others.

However, as the application space for synthetic biological programs expands, there is a growing need for more resource-efficient biological programming structures that enable an increase in circuit complexity to meet point-of-use functionality better. This need is at least partially fulfilled by the present disclosure.

### SUMMARY

Disclosed herein are quantum-inspired gene circuits for a synthetic biological program, the circuit being capable of multiple potential computational states in parallel through stochastic, combinatorial, or probabilistic biochemical interactions that leverage and emulate an abstraction of reversible logic operations using the gene circuits in combination with bi-directional promoters.

In one aspect disclosed herein is a gene circuit capable of synthetic biological program operations comprising a quantum logic gate, wherein the quantum logic gate comprises at least one input, a bi-directional promoter, at least two synthetic transcription factors, and at least one cognate synthetic promoter. In some embodiments, the synthetic biological program operation is a biological QUBIT or PAULI-X logic gate. In a further embodiment, one input (such as, for example, isopropyl β-D-1-thiogalactopyranoside (IPTG), D-ribose, fructose, or cellobiose) results in two outputs. In an additional embodiment, the bi-directional promoter comprises SEQ ID NO: 9, SEQ ID NO: 64, SEQ ID NO: 66, or a sequence at least 90% identical to SEQ ID NO: 9, SEQ ID NO: 64, or SEQ ID NO: 66. In some embodiments, the at least two synthetic transcription factors comprise a repressor and an anti-repressor. In another embodiment, the gene circuit comprises at least one additional quantum logic gate layered to engineer additional synthetic biological program operations having quantum-inspired programming logic gates (such as, for example, FEYNMAN or TOFFOLI gate) of increasing complexity. In a further embodiment, two inputs (such as, for example, isopropyl β-D-1-thiogalactopyranoside (IPTG), D-ribose, fructose, or cellobiose) result in four outputs. In an even further embodiment, the gene circuit performs a swap-of-function memory operation, performed by at least one recombinase, wherein the regulatory control of the output channels is swapped.

In another aspect, disclosed herein is a bi-directional promoter comprising SEQ ID NO: 9, SEQ ID NO: 64, SEQ ID NO: 66, or a sequence at least 90% identical to SEQ ID NO: 9, SEQ ID NO: 64, or SEQ ID NO: 66.

In an additional aspect, disclosed herein, is a cell comprising a plurality of nucleic acid sequences, encoding a gene circuit comprising a bi-directional promoter, at least two synthetic transcription factors responsive to input, at least two cognate synthetic promoters, and at least two outputs. In some embodiments, the cell is a bacterial cell. In a further embodiment, the plurality of nucleic acid sequences are operably linked. In additional embodiments, the bi-directional promoter comprises SEQ ID NO: 9, SEQ ID NO: 64, SEQ ID NO: 66, or a sequence at least 90% identical to SEQ ID NO: 9, SEQ ID NO: 64, or SEQ ID NO: 66. In a further embodiment, the at least two synthetic transcription factors responsive to input are a repressor and an anti-repressor.

### BRIEF DESCRIPTION OF FIGURES

The accompanying drawings, which are incorporated and constitute part of this specification, illustrate several embodiments and together with the description illustrate the disclosed compositions and methods.

FIGS. 1A-1G show the design of a fully compressed biological QUBIT logic operation. FIGS. 1A-1B show complementary quantum logical operations for developing biological QUBIT logic. The biological QUBIT gate is comprised of (i) a logical BUFFER operation with the synthetic LacI transcription factor I⁺KSL regulating the OUTPUT RFP (FIG. 1A), and (ii) a logical NOT operation with the synthetic anti-LacI transcription factor I^{A(9)}YQR regulating the OUTPUT GFP (FIG. 1). Gate diagrams, genetic architectures, and truth tables are shown. FIG. 1C is a gate diagram showing the concatenation of BUFFER and NOT logic operations via INPUT coupling (i.e., combination of both operations in a single cell) to perform biological QUBIT logic. FIG. 1D is a gate diagram showing the concatenation of BUFFER and NOT logic operations via INPUT coupling to perform biological PAULI-X logic (i.e., antithetical to the QUBIT operation). FIGS. 1E-1F show the genetic architecture designs for biological QUBIT logic. FIG. 1E shows an inverter-based QUBIT circuit, while FIG. 1F shows a partially compressed QUBIT circuit that utilizes an anti-repressor to eliminate the inverter, and FIG. 1G shows a fully compressed QUBIT circuit that leverages an anti-repressor and a bi-directional promoter. The inverter-based QUBIT requires the most resources, 3 RNA polymerases+3 transcription factors. The partially compressed QUBIT requires fewer resources, 2 RNA polymerases+2 transcription factors. Notably, the fully compressed iteration of the QUBIT operation requires the fewest number of resources, 1 RNA polymerase+2 transcription factors. The design of the fully compressed QUBIT is composed of a single bi-directional promoter driving the transcription of GFP and RFP, which are regulated as described in FIGS. 1A-1B—both transcription factors recognize a single INPUT (i.e., IPTG) yet perform opposite regulatory phenotypes upon induction. The legend describes the iconography used in circuit designs.

FIGS. 2A-2D depict bi-directional promoter architectures and their regulatory performance. FIG. 2A shows DNA elements of engineered bi-directional promoters. Green regions represent promoter elements driving GFP expression, and red regions represent promoter elements driving RFP expression. Brown regions represent promoter elements driving both GFP and RFP expression. FIG. 2B shows the regulatory performance of each bi-directional promoter with (i) IPTG BUFFER logic via I⁺KSL regulation of RFP, (ii) IPTG NOT logic via I^{A(9)}YQR regulation of GFP, or (iii) constitutive expression with no transcription factor. Each box represents a GFP (green) or RFP (red) OUTPUT value for each promoter configuration, regulatory element, and ligand state. FIG. 2C shows the crystal structure of the wild-type LacI tetramer (PDB: 1LBI), used as a visual representation of I⁺KSL and I^{A(9)}YQR transcription factors, with mutations conferring the I^{A(9)}YQR phenotype highlighted in dark blue. Performance of the IPTG biological QUBIT logical operation. Both I⁺KSL and I^{A(9)}YQR were co-expressed and regulated the 2-OUTPUT reporter system with the P^{BD(35)}rep promoter configuration. Note that the OFF state of GFP is considerably leakier than the system in FIG. 2B with I^{A(9)}YQR only. FIG. 2D shows the crystal structures shown in FIG. 2C with I⁺KSL L251A and I^{A(9)}YQR L349* mutations highlighted in red, and performance of IPTG biological QUBIT with I⁺KSL L251A and IA(9)YQR L349* to restrict heterotetrameric and heterodimeric protein assemblies. The GFP and RFP OFF states decreased by 6.75× and 1.6× from those in FIG. 2C, respectively. In all experiments, GFP and RFP OUTPUT fluorescence measurements were normalized to maximum values of 15,000 R.F.U. and 300 R.F.U., respectively, and are shown here on a fractional scale. Crystal structure images were generated using 3D Protein Imaging.

FIGS. 3A-3D depict biological QUBIT and PAULI-X quantum logic gates. Gate diagram, truth table, genetic architecture, dose response, and state response of (FIG. 3A) IPTG biological QUBIT logical operation employing I⁺KSL L251A and I^{A(9)}YQR L349* transcription factors, (FIG. 3B) D-ribose biological QUBIT logical operation employing R⁺KSL and R^{A(2)}YQR transcription factors, (FIG. 3C) IPTG biological PAULI-X logical operation employing I^{A(6)}KSL L349* and I⁺YQR L251A transcription factors, and (FIG. 3D) D-ribose biological PAULI-X logical operation employing R^{A(2)}KSL and R⁺YQR transcription factors. The 2-OUTPUT reporter with the P^{BD(35)}rep promoter configuration was used for all quantum logic gates. The data represent six biological replicates per experiment. Dose-response and state-response data were obtained through separate experiments conducted on different days. Dose-response data were fit with a Hill function curve (parameters and statistics are given in the data herein).

FIGS. 4A-4F depict biological FEYNMAN and TOFFOLI quantum logic gates. FIG. 4A shows a biological IPTG-D-ribose (IR) FEYNMAN quantum logic gate diagram and truth table. The OUTPUT state is flipped when INPUT 1 (IPTG) is present. FIG. 4B shows the genetic architecture of the IR FEYNMAN quantum logic gate. IPTG-inducible transcription factors I⁺YQR L251A and I^{A(9)}HQN L349* regulate a D-ribose-inducible transcription factor layer comprising R+KSL and RA(2)KSL, driven by the P^{BD(35)}lac1 bi-directional promoter and regulated by D-ribose transcription factors that FEEDFORWARD to regulate the 2-OUTPUT P^{BD(35)}rep reporter. FIG. 4C shows the performance of the IR FEYNMAN quantum logic gate. INPUT conditions (rows) and OUTPUT expression (columns) are shown for GFP (green) and RFP (red). Numerical values represent the OUTPUT measurement on a fractional scale. FIG. 4D shows a biological Cellobiose-IPTG-D-ribose (EIR) TOFFOLI quantum logic gate diagram and truth table. The OUTPUT state is flipped when both INPUT 1 (Cellobiose) and INPUT 2 (IPTG) are present. FIG. 4E shows the genetic architecture of the EIR TOFFOLI quantum logic gate. The cellobiose-inducible repressor E⁺TAN regulates both I^{A(9)}HQN L349* and R^{A(2)}KSL anti-repressors, as well as a BFP (control) OUTPUT on the reporter. Regulated I^{A(9)}HQN L349* and I⁺YQR L251A regulate R⁺KSL and R^{A(2)}KSL, respectively, which FEEDFORWARD to regulate GFP and RFP. The P^{BD(35)}lac0 and P^{BD(35)}lac1 bi-directional promoters drive the transcription of IPTG-inducible transcription factors I⁺YQR L251A and IA(9)HQN L349, respectively, and D-ribose-inducible transcription factors R⁺KSL and RA(2)YQR. FIG. 4F shows the performance of the EIR TOFFOLI quantum logic gate. INPUT conditions (rows) and OUTPUT expression (columns) are shown for GFP (green), RFP (red), and BFP (blue).

FIGS. 5A-5D depict an engineered 4-OUTPUT circuit using biological QUBIT gates. FIG. 5A shows a diagram of a 4-OUTPUT circuit. IPTG and D-Ribose QUBIT gates are used to regulate four fluorescent reporter OUTPUTs—GFP, RFP, BFP, and YFP—with NOR, A NIMPLY B, B NIMPLY A, or AND logic, respectively. FIG. 5B shows a truth table, and FIG. 5C shows the genetic architecture of the 4-OUTPUT circuit. IPTG QUBIT gate transcription factors I⁺KSL L251A and I^{A(9)}YQR L349* and D-Ribose QUBIT gate transcription factors R^{A(2)}NAR and R⁺HQN are constitutively expressed and regulate each of the 4 OUTPUTs on the reporter. Each OUTPUT is expressed from P^{BD(35)}rep bi-directional promoters analogous to the QUBIT architectures shown previously, with the modification of orthogonal operator combinations upstream of each OUTPUT. This modification enables the full development of the abstract quantum logic gate scheme shown in FIG. 5A and the corresponding truth table given in FIG. 5B. FIG. 5D shows the performance of a 4-OUTPUT circuit. The fluorescence of each OUTPUT is indicated for each INPUT combination. Mean values are normalized to a scale of 1 for each OUTPUT and displayed on the plot.

FIGS. 6A-6E depict recombinase-based QUBIT to PAULI-X gate Swap-of-Function (SoF) memory operation. FIG. 6A shows an example of a conventional decision-making system comprised of a single logical BUFFER operation. When the INPUT (black hexagon) is added or removed, the OUTPUT state reversibly changes from ON to OFF, respectively. An example of a recombinase-mediated memory-only system composed of a single gain-of-function (GOF) operation is shown. When the INPUT (blue hexagon) is added to the system, the OUTPUT state permanently changes from OFF to ON via DNA recombination. FIG. 6B shows a diagram of the QUBIT to PAULI-X gate conversion operation, which offers cooperative decision-making and memory functions. A biological QUBIT gate, which performs reversible OUTPUT state change (i.e., GFP to RFP) upon addition of the decision-making INPUT, is transformed to a PAULI-X gate via the addition of a memory INPUT. The resulting PAULI-X gate performs the reverse OUTPUT state change (i.e., RFP to GFP) upon addition of the decision-making INPUT. FIG. 6C shows the genetic architecture of QUBIT to PAULI-X gate conversion memory operation. The transcription factor PhlF is expressed constitutively and regulates the recombinase A118. Additionally, a repressor (X⁺KSL) and an anti-repressor (XAYQR) regulate—i.e., X=I or R—the PBD(35)rep inducible promoter, which is flanked by A118 recombinase attB and attP attachment sites. Upon PhlF induction (with DAPG), A118 is produced and inverts the P^{BD(35)}rep promoter and operator DNA element, inverting the regulatory control of each OUTPUT and converting the biological QUBIT gate to a biological PAULI-X gate. FIG. 6D shows the performance of IPTG and D-ribose biological QUBIT gates. Each OUTPUT was measured with a plate reader (bars) and flow cytometry (histograms). Following these measurements, the memory operation was performed, and the circuits were reassayed under the same conditions. FIG. 6E shows the memory of biological PAULI-X logic (converted QUBIT gates) as described in FIG. 6D.

FIG. 7 depicts the genetic parts used in 2-INPUT FEYNMAN gates. Tables containing promoters, operators, insulators, RBSs, terminators, and plasmids used for expression cassettes of each gene in IPTG-D-ribose biological FEYNMAN and D-ribose-IPTG biological FEYNMAN quantum logic gates. Sequences of each genetic part are given in Tables 1-6.

FIGS. 8A-8C depict the putative interaction of RNA polymerase with the bidirectional promoter. RNA polymerase can bind to the bi-directional promoter in either of two directions: (FIG. 8A) the forward direction to transcribe GFP, or (FIG. 8B) the reverse direction to transcribe RFP. (FIG. 8C) Two or more RNA polymerase complexes are sterically hindered from simultaneously binding to the bidirectional promoter in forward and reverse directions.

FIG. 9 depicts maps of the INPUT states to the OUTPUT states in a 2-QUBIT 4-OUTPUT circuit. The 4-OUTPUT circuit contains the p15a plasmid for constitutive expression of four transcription factors (constituting IPTG and D-Ribose QUBIT gate regulators), as well as the pSC101 plasmid for regulated expression of four fluorescent OUTPUTs (GFP, RFP, BFP, and YFP). INPUT conditions are mapped to OUTPUT states with the putative interaction of regulators and reporter shown at the protein-DNA level. Colored dashed boxes highlight the OUTPUT in the ON state across each INPUT condition, wherein each regulator is unbound from its cognate DNA operator.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### DEFINITIONS

As used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, the “less than or equal to 10” as well as “greater than or equal to 10” is also disclosed. It is also understood that throughout the application, data is provided in a number of different formats, and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition, or activity. An increase can be any individual, median, or average increase in a condition, symptom, activity, or composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase, so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, or composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease, so long as the decrease is statistically significant.

“Inhibit,” “inhibiting,” and “inhibition” mean to decrease an activity, response, condition, disease, or other biological parameter. This can include, but is not limited to, the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between, as compared to native or control levels.

By “reduce” or other forms of the word, such as “reducing” or “reduction,” is meant the lowering of an event or characteristic (e.g., tumor growth). It is understood that this is typically in relation to some standard or expected value; in other words, it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control, as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Comprising” is intended to mean that the compositions, methods, etc., include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate-buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “promoter,” as used herein, refers to a sequence in DNA that mediates the initiation of transcription by an RNA polymerase. Transcription promoters may comprise one or more of a number of different sequence elements as follows: 1) sequence elements present at the site of transcription initiation; 2) sequence elements present upstream of the transcription initiation site; and 3) sequence elements downstream of the transcription initiation site. The individual sequence elements function as sites on the DNA where RNA polymerases and transcription factors that facilitate the positioning of RNA polymerases on the DNA bind. A “bidirectional promoter” is a regulatory DNA sequence that is capable of initiating transcription in two opposite (divergent) directions, thereby driving expression of two distinct genes.

A “transcription factor” refers to a sequence-specific DNA-binding protein that controls the rate of transcription of genetic information from DNA to messenger RNA by binding to a specific DNA sequence; this specific DNA sequence is referred to as the cognate binding sequence, and the transcription factor is called the cognate transcription factor.

As used herein, a “transcription terminator” or a “terminator” refers to a segment of a nucleic acid sequence that marks the end of a gene in genomic DNA during the transcription process, or gene expression. This sequence mediates or signals the end of transcription by providing signaling nucleotides in newly synthesized RNA transcripts that trigger an RNA polymerase to release the DNA and newly synthesized RNA.

The word “vector” refers to any vehicle that carries a polynucleotide into a cell for the expression of the polynucleotide in the cell. The vector may be, for example, a plasmid, a virus, a phage particle, or a nanoparticle. A “bacterial plasmid” is a small extrachromosomal DNA molecule that can be incorporated into another cell that is physically separated from the chromosomal DNA and is easily replicated. Once transformed into a suitable host, the vector may replicate and function independently of the host genome, or may, in some instances, integrate into the genome itself. In some embodiments, the vector is a DNA construct containing a DNA sequence which is operably linked to a suitable control sequence capable of affecting the expression of the DNA in a suitable host cell. Such control sequences can include a promoter to effect transcription, an optional operator sequence to control such transcription, a sequence encoding suitable mRNA ribosome binding sites, and sequences that control the termination of transcription and translation.

An “effective amount” is an amount sufficient to affect beneficial or desired results. An effective amount can be administered in one or more administrations, applications, or dosages.

“Genetic circuits” are comprised of a set of heterologous expression cassettes whose (generally protein) products regulate other expression cassettes in the set and/or regulate an ultimate output of the circuit. Genetic circuit components can be used to implement logic operations in living cells based on an input detected by the circuit. Individual components for particular program operations can be coupled to inputs and to one another in order to implement a genetic circuit that operates on a complex expression. Genetic circuits may connect logic variables representing the cues via logic operations. Quantum logic gates can be generated using expression cassettes that respond to biological inputs, wherein the expression cassettes are regulated using combinations of repressors and activators.

The terms “gate”, “logic gate”, or “quantum logic gate” are used to refer to a device or molecular mechanism that produces a particular (predetermined) output in response to one or more inputs. Quantum logic gates and their uses are well known to those of skill in the art (see, e.g., Horowitz and Hill (1990), The Art of Electronics, Cambridge University Press, Cambridge).

A “nucleotide” is a compound consisting of a nucleoside, which consists of a nitrogenous base and a 5-carbon sugar, linked to a phosphate group forming the basic structural unit of nucleic acids, such as DNA or RNA. The four types of nucleotides are adenine (A), cytosine (C), guanine (G), and thymine (T), each of which is bound together by a phosphodiester bond to form a nucleic acid molecule.

A “nucleic acid” is a chemical compound that serves as the primary information-carrying molecules in cells and makes up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA).

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences and, therefore, achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences,” which is used for direct pairwise comparison of two nucleotide sequences. “BLAST 2 Sequences” can be accessed and used interactively at the NCBI website. The “BLAST 2 Sequences” tool can be used for both Blastn and Blastp.

Percent identity may be measured over the length of an entire defined polynucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

A “full length” polynucleotide sequence is one containing at least a translation initiation codon (e.g., methionine) followed by an open reading frame and a translation termination codon. A “full length” polynucleotide sequence encodes a “full length” polypeptide sequence.

As used herein, “upstream” refers to the relative position of a genetic sequence, either DNA or RNA. Upstream relates to the 5′ to 3′ direction relative to the start site of transcription, wherein upstream is usually closer to the 5′ end of a genetic sequence.

As used herein, “downstream” refers to the relative position of a genetic sequence, either DNA or RNA. Downstream relates to the 5′ to 3′ direction relative to the start site of transcription, wherein downstream is usually closer to the 3′ end of a genetic sequence.

“Gene” includes a nucleic acid fragment that expresses a functional molecule such as, but not limited to, a specific protein, including regulatory sequences preceding (5′ noncoding sequences) and following (3′ non-coding sequences) the coding sequence. “Native gene” refers to a gene as found in its natural endogenous location with its own regulatory sequences.

By “domain” it is meant a contiguous stretch of nucleotides (that can be RNA, DNA, and/or RNA-DNA-combination sequence) or amino acids.

An “enhancer” is a DNA sequence that can stimulate promoter activity and may be an innate element of the promoter or a heterologous element inserted to enhance the level or tissue-specificity of a promoter. Promoters may be derived in their entirety from a native gene or be composed of different elements derived from different promoters found in nature and/or comprise synthetic DNA segments. It is understood by those skilled in the art that different promoters may direct the expression of a gene in different tissues or cell types, at different stages of development, or in response to different environmental conditions. It is further recognized that since in most cases the exact boundaries of regulatory sequences have not been completely defined, DNA fragments of some variation may have identical promoter activity.

As used herein, the term “operably linked” refers to a functional relationship between two or more nucleic acid sequences wherein the sequences are arranged relative to one another such that at least one of the sequences is capable of exerting a desired biological effect on another sequence.

### Biological Computation

Biological computation, at its core, is the ability to engineer and develop systems capable of converting information (inputs) into a programmable gene expression (output(s)). Gene regulation in biological systems can be viewed as a molecular computer. That is, gene expression can be modeled as on-off states of Boolean (digital) logic, which can integrate multiple digital inputs into a desired output. Furthermore, the present disclosure teaches that gene expression can also be modeled as quantum logic, wherein outcomes are context-dependent, non-binary states. Quantum gene logic allows multiple expression states.

Quantum computing refers to a computational paradigm in which informational processing is performed using system states that represent and evolve according to multiple concurrent logical configurations, with outcomes determined by superposition, interaction, and read-out. In the present disclosure, quantum computing in the context of gene circuits refers to an information-processing framework within or modeled by biological gene circuits, wherein genetic, epigenetic, or biochemical states are configured to function as multi-state computational units capable of representing, processing, and resolving multiple potential computational states in parallel through stochastic, combinatorial, or probabilistic biochemical interactions. Quantum computing is not necessarily quantum mechanical; instead, it leverages and emulates an abstraction of reversible logic operations in the form of QUBITs, where a system can represent not just one possibility, but multiple possibilities—commonly referred to as a superposition. In the context of a fundamental QUBIT operation, each INPUT state maps to a unique dual-state OUTPUT—i.e., where the OUTPUT vector is |0 [symbol] =[1,0] when the INPUT is 0, and is |1 [symbol] =[0,1] when the INPUT is 1.

Biological computation enables living or molecular systems to process information, make decisions, and control outcomes in applications spanning medicine, manufacturing, sensing, materials, data storage, and intelligent systems.

### Function as Memory State

A memory operation can alter an encoded functional (information-bearing) state. Loss-of-function, gain-of-function, and swap-of-function are memory operations applicable to gene circuits. In a loss-of-function memory operation, a previously encoded functional capability or state is reduced or eliminated, resulting in a partial or complete erasure of the function. Loss-of-function operations do not require the creation of new functionality. In a gain-of-function memory operation, a system acquires a new or enhanced function, thereby increasing the information content encoded in memory. A swap-of-function is an operation in which an existing functional state is replaced with a different functional state, such that the system's overall functional role is altered without a net change in the number of stored functions. In some embodiments, the disclosed gene circuit performs a swap-of-function memory operation. This swap-of-function memory operation is performed by at least one recombinase.

### Compositions

Disclosed are the components to be used to prepare the disclosed compositions. It is understood that when combinations, subsets, interactions, groups, etc. of these materials are disclosed, that while specific reference to each various individual and collective combinations and permutations of these compounds may not be explicitly disclosed, each is specifically contemplated and described herein.

Disclosed herein is a gene circuit capable of synthetic biological program operations comprising a quantum logic gate, wherein the quantum logic gate comprises at least one input, a bi-directional promoter, at least two synthetic transcription factors, and at least one cognate synthetic promoter. In some embodiments, one input (such as, for example, isopropyl 3-D-1-thiogalactopyranoside (IPTG), D-ribose, fructose, or cellobiose) results in two outputs. The synthetic biological program operation can be a biological QUBIT or PAULI-X quantum logic gate. In some embodiments, when at least two quantum logic gates are layered, wherein at least two inputs result in four outputs, the biological program operation can be a FEYNMAN or TOFFOLI gate. At least 2, 3, 4, 5, 6, 7, 8, 9, 10, or more quantum logic gates can be layered to engineer synthetic biological program operations of increasing complexity. The synthetic transcription factors can be responsive to input (such as, for example, isopropyl 3-D-1-thiogalactopyranoside (IPTG), D-ribose, fructose, or cellobiose) and can comprise repressors and anti-repressors.

Also disclosed herein is a bi-directional promoter comprising SEQ ID NO: 9, SEQ ID NO: 64, SEQ ID NO: 66, or a sequence at least 80%, 85%, 90%, 91%, 92%, 93%, 94%, 95%, 96%, 97%, 98%, or 99% identical to SEQ ID NO: 9, SEQ ID NO: 64, or SEQ ID NO: 66.

Further disclosed is a cell comprising a plurality of nucleic acid sequences, encoding the disclosed gene circuit. In some embodiments, the cell is a bacterial cell.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### EXAMPLES

The following examples are put forth so as to provide those of ordinary skill in the art with a complete disclosure and description of how the compounds, compositions, articles, devices, and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in °C or is at ambient temperature, and pressure is at or near atmospheric.

### Example 1: Quantum-Inspired Logic for Advanced Transcriptional Programming

One way to achieve greater programming efficiency is via circuit compression—i.e., reducing the number of parts used to construct a given genetic circuit or genetic operation. To this end, Transcriptional Programming (T-Pro) has emerged as the state-of-the-art regarding circuit compression [9], [10], [13]. T-Pro leverages modular sets of synthetic transcription factors and cognate synthetic promoters to achieve circuit compression via networked functions. A fundamental advantage of this toolkit is the use of two classes of synthetic transcription factors: (i) repressors and (ii) anti-repressors, which can be abstracted to BUFFER and NOT logic gates, respectively. When repressors and anti-repressors are employed to regulate a user-defined genetic architecture composed of cognate synthetic promoters, T-Pro can form complete sets of compressed Boolean logical operations—analogous to the fundamental decision-making units of a computer program.

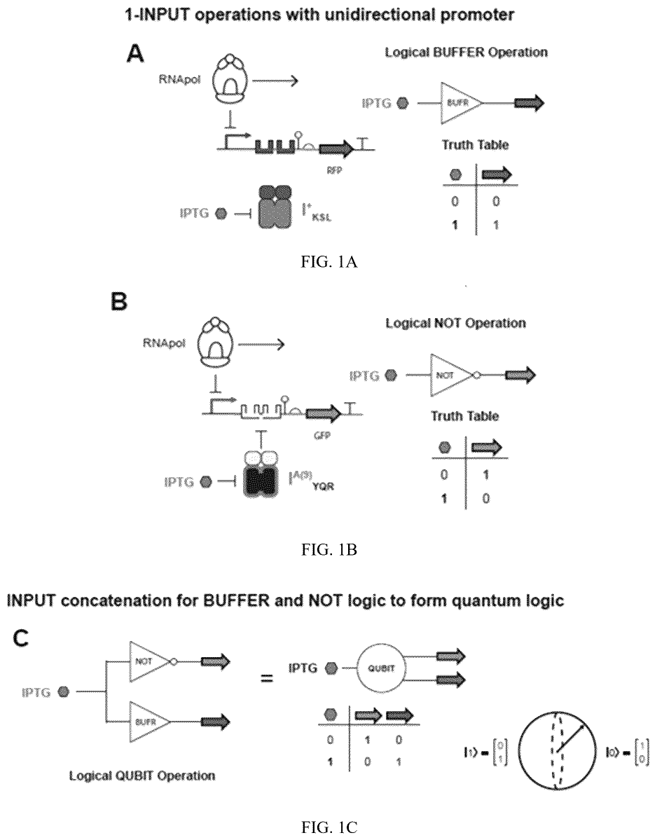

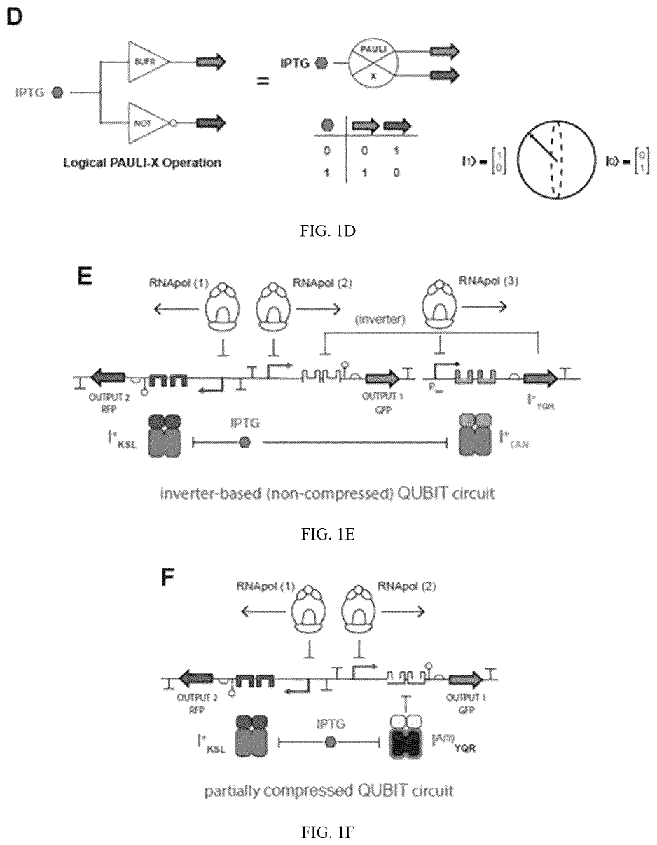

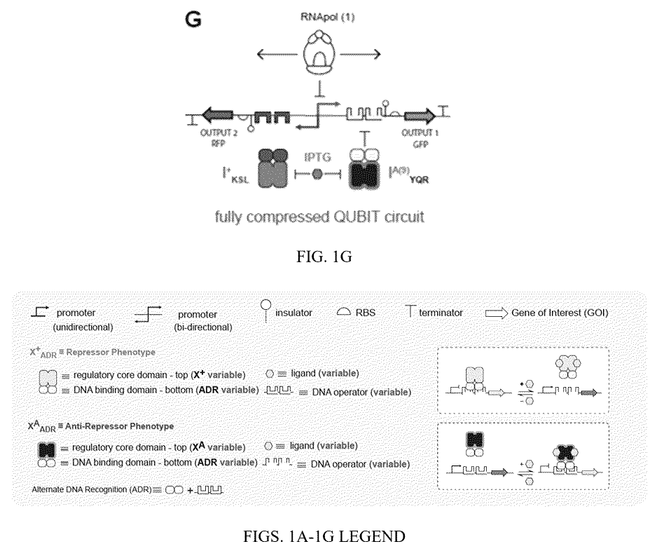

The study posits that T-Pro can be extended beyond standard Boolean logic to emulate information transfer observed in quantum computing systems [32]. A key advantage of quantum programming is logical reversibility, wherein each OUTPUT state can be mapped to a specific INPUT state and vice-versa—a distinguishing feature relative to classical bit (binary) programming. Quantum computing reversible logic should not be conflated with thermodynamic reversibility—see Note 1. Quantum computing is not necessarily quantum mechanical; instead, it leverages and emulates an abstraction of reversible logic operations in the form of QUBITs, where a system can represent not just one possibility, but multiple possibilities—commonly referred to as a superposition. In this study, the objective is to design analogous quantum logical operations in the context of biological circuits. Namely, the study aims to design a fundamental biological QUBIT logical operation in which each INPUT state maps to a unique dual-state OUTPUT—i.e., where the OUTPUT vector is |0 [symbol] =[1,0] when the INPUT is 0, and is |1 [symbol] =[0,1] when the INPUT is 1 (see FIGS. 1A-1C). Ideally, the resulting biological QUBIT is entangled with other biological unit operations—i.e., in the sense that multiple biological program operations are correlated with a high probability, enabling the systematic development of multi-INPUT multi-OUTPUT biological programs with more efficient INPUT economy.

In quantum computing systems, reversible logic is utilized to reduce calculation cost (i.e., the number of gates used to construct a program) and the number of OUTPUT states which are not the primary OUTPUT or that advance the computation [33], [34]. In practice, programs predicated on quantum reversible logic can modulate two or more OUTPUTs with fewer INPUTs compared to classical systems. Axiomatically, fundamental units of reversible logic can be layered (or entangled) to form operations of higher complexity. In principle, quantum (reversible) logic applied to biological systems has the potential to dramatically reduce the number of genetic parts required for a given circuit, analogous to abiotic systems. Said technology could be used in multi-product biomanufacturing, branched-pathway metabolic engineering applications, or any non-linear multistep biological process wherein multiple OUTPUT regulation benefits from efficient INPUT economy.

Many reversible logic gates have been developed abiotically using superconducting devices [35], via optics [36], [37], enzymatically [38], [39], and recently in living systems by way of a community-level platform [40], [41]. However, the biological (biotic) iterations of said logic gates do not operate at a single-cell level and require a population to measure, which is prone to instability due to community-level fluctuations. Only one example of a single-cell reversible gate has been reported [42]. This system is constructed with two duplicate GFP OUTPUTs to achieve an apparent FEYNMAN gate truth table; therefore, the operation cannot be technically classified as a reversible gate regarding the true transcriptional OUTPUTs. To facilitate open-ended synthesis of efficient and complex genetic circuits, a modular platform for constructing reversible logic is needed. The study posits that T-Pro can serve as a platform technology for this purpose.

The present disclosure presents a novel T-Pro biocomputing circuit inspired by quantum computing principles using Escherichia coli (E. coli) as the chassis cell. This technology is achieved via (i) engineering novel synthetic bi-directional promoters that facilitate transcription of a dual-state OUTPUT, and (ii) developing single-INPUT transcriptional control of the OUTPUT state via complementary synthetic repressor and anti-repressor transcription factors from the T-Pro toolkit. This study demonstrates that this platform is modular and scalable from 1-INPUT operations to 2-INPUT, 3-INPUT, and 4-INPUT reversible logic programs. Commensurate with the supposition, said operations increased information transfer in genetic circuits by purposing a greater portion of the INPUT permutation space for reversible logic OUTPUTs. Furthermore, the study demonstrated that this new platform technology seamlessly pairs with complementary memory operations [43], [44], demonstrated as truth table remapping between two fundamental biological program operations in situ. This platform technology represents a paradigm shift in biocomputing, advancing the current state of genetic programming technology by enabling and expansion of efficient computation in carbon that leverages the systemization of Transcriptional Programming.

### Materials & Methods

Cloning, Strains, and Media. Transcription factors and recombinases were expressed on the pLacI plasmid (Novagen) which contains the p15a origin (copy number 20-30/cell), and output genes (GFP=sfGFP, RFP=mCherry, BFP=tagBFP, YFP=phiYFP) were expressed on the pZS*22-sfGFP reporter plasmid which contains the pSC101 origin (copy number 3-5/cell). Chloramphenicol and kanamycin resistance genes were used as selection markers for transcription factor and reporter plasmids, respectively. Output genes were assembled into reporter plasmids using Golden Gate Assembly (BsaI-HF v2 Golden Gate Assembly Kit, NEB). Transcription factors were first subcloned into pUC19 vector carrying an ampicillin resistance gene (NEB) with BsmBI restriction sites, grown in LB with carbenicillin, sequence confirmed with Sanger DNA sequencing (Eurofins Genomics), and then assembled into the pLacI plasmid using Golden Gate Assembly (BsmBI-HF v2 Golden Gate Assembly Kit, NEB). A pLacI destination plasmid with PhlF and A118 was constructed using Gibson Assembly (NEBuilder HiFi DNA Assembly Master Mix, NEB) for generating the memory transcription factor plasmids. All promoter, operator, insulator, RBS, and terminator sequences were cloned using site-directed mutagenesis PCR (Q5 DNA Polymerase, NEB) with custom primers (Eurofins Genomics), followed by kinase, ligase, and DpnI reactions (KLD enzyme mix, NEB) prior to final construct assemblies. All promoter, operator, insulator, RBS, terminator, and coding sequences can be found in Tables 1-6. All cloning plasmids in this work were transformed into chemically competent DH5α cells (huA2 Δ(argF-lacZ)U169 phoA glnV44 φ80Δ(lacZ)M15 gyrA96 recA1 relA1 endA1 thi-1 hsdR17; New England Biolabs) and plated on LB agar with appropriate antibiotics. The resulting transformants were cultured overnight in LB Broth with the appropriate antibiotics and then miniprepped (Omega Bio-Tek) to yield each plasmid. The sequence was confirmed using NGS Whole Plasmid DNA sequencing (Eurofins Genomics).

Transcriptional Logic Gate Experiments. Transcription factor and corresponding reporter plasmids were double transformed into homemade chemically competent 3.32 E. coli cells (Genotype lacZ13(Oc), lacI22, LAM-, e14-, relA1, spoT1, and thiE1, Yale CGSC #5237) and six individual transformants (biological replicates) were precultured for 6 hours in LB media with chloramphenicol (25 μg/mL, VWR Life Sciences) and kanamycin (35 μg/mL, VWR Life Sciences) antibiotics. Precultures were then diluted into glucose (100 mM, Fisher Scientific) M9 minimal media supplemented with 0.2% (w/v) casamino acids (VWR Life Sciences), 1 mM thiamine HCl (Alfa Aesar), antibiotics, and respective inducers, and grown in a flat bottom 96-well microplate (Costar) for 16 hours (37 °C, 300 rpm). Microwell plates were sealed with Breathe-Easy membranes (Diversified Biotech) to prevent evaporation. The inducer concentrations used are as follows: isopropyl-β-D-thiogalactoside (IPTG, 10 mM), D-ribose (10 mM), and cellobiose (10 mM). Optical density (OD600), GFP fluorescence (*_{!″}=485 nm, *_{!#}=510 nm, Gain 400), RFP fluorescence (*_{!″}=585 nm, *_{!#}=610 nm, Gain 800), BFP fluorescence (*_{!″}=399 nm, *_{!#}=454 nm, Gain 400), and YFP fluorescence (*_{!″}=517 nm, *_{!#}=557 nm, Gain 400) were measured with a Spectramax M2e plate reader (Molecular Devices). The LacSTOP control plasmid was also assayed with each reporter construct to determine the maximum expression level of each genetic architecture. Measurements were corrected by subtracting values of blank media from sample values, and fluorescence values were normalized to optical density in Microsoft Excel (Microsoft) and divided by a global maximum (per fluorescent channel) to obtain the Fraction of Maximum Output (F.M.O). For 1-input and 2-input gate plots with a single y-axis scale, fluorescence values were divided by the maximum of the 2-output reporters: 15,000 relative fluorescence units (R.F.U.) for GFP and 300 R.F.U. for RFP. A built-in log(agonist) vs. normalized response model in GraphPad Prism was used to fit the experimental dose-response data, and the fitting parameters are provided in the data presented herein. For the 3-input TOFFOLI gate, fluorescence values were divided by the maximum values of the 3-output reporters: 6,000 R.F.U. for GFP, 200 R.F.U. for RFP, and 2,500 R.F.U. for BFP. For the 4-output gate, fluorescence values were divided by the reporter maximum of 1,500 R.F.U. for GFP, 100 R.F.U. for RFP, 1,500 R.F.U. for BFP, and 1,500 R.F.U. for YFP. The resulting data were plotted using GraphPad Prism.

Library Generation and Screening. Promoter and RBS libraries were generated using site-directed mutagenesis as described above with primers containing N (A, T, C, or G), R (A or G), or S (G or C) nucleotides (Eurofins Genomics) to constitute the resulting library space. Library mutagenesis reactions were performed on individual transcription factors subcloned in pUC19 and transformed into chemically competent DH5α cells as described above. The recovery was outgrown in LB with carbenicillin overnight and miniprepped. The resulting DNA library subclones were subsequently used in Golden Gate Assemblies to generate IR FEYNMAN, RI FEYNMAN, and EIR TOFFOLI gate libraries. Following assembly, the libraries were transformed, diluted, and plated to calculate library size. They were then outgrown in LB with chloramphenicol and subsequently miniprepped. The assembled DNA libraries were then double-transformed with the corresponding reporter plasmids into 3.32 E. coli as described above. Individual colonies were picked for screening and assayed. The top 7 performing clones were then streaked onto LB agar containing chloramphenicol and kanamycin antibiotics and reassayed with six biological replicates, as described previously. Top-performing variants were then grown in LB with antibiotics, miniprepped, and sequenced.

Memory (Recombinase) Experiments. Circuit plasmids containing A118, regulated by PhlF and either IPTG or D-ribose-inducible transcription factors, were transformed into 3.32 E. coli cells and plated on LB agar with 25 μg/mL chloramphenicol. A transformant containing each type of memory plasmid (i.e., IPTG QUBIT, IPTG PAULI-X, D-ribose QUBIT, or D-ribose PAULI-X) was cultured in LB with antibiotics and used to generate chemically competent cells. The resulting competent cells were transformed with the memory reporter plasmid, which contains A118 attachment sites. Six transformants (biological replicates) were then precultured for 6 hours in LB medium supplemented with chloramphenicol and kanamycin antibiotics. Precultures were diluted into glucose M9 minimal media and assayed as described above. Assay cultures were then diluted 1:100 into (1) PBS containing 10× kanamycin (350 μg/mL) for flow cytometry analysis, and (2) fresh LB medium containing 10 μM 2,4-diacetylphloroglucinol (DAPG) and the appropriate antibiotics. Cultures were grown in a 96-well microplate at 37 °C and 300 rpm, sealed with a Breathe-Easier membrane (Diversified Biotech), for 8 hours to allow for recombination. Cultures were then diluted into glucose M9 minimal media with appropriate inducer conditions and assayed again as described above. Assay cultures were diluted into PBS for flow cytometry analysis.

Flow cytometry. Flow cytometry analysis was performed with a Cytoflex S (Beckman Coulter) flow cytometer. After the memory (recombinase-based) experiments, cells were diluted 1:40 into PBS with 2 mg/mL of kanamycin and incubated for an hour at room temperature. Cells were then analyzed at a flow rate of 20-30 L/min and gated by forward scatter (FSC) versus side scatter (SSC) to eliminate cell debris. To gate the correct cell population, events were further gated by side scatter height vs. side scatter area. Fluorescence was monitored through the FITC channel for GFP expression and the ECD channel for RFP expression. More than 20,000 events were collected and analyzed by CytExpert (Version 2.5) software. All flow cytometry assays had 6 biological replicates.

### Results

Engineering a 2-OUTPUT synthetic promoter for QUBIT logic. Inspired by the function of the QUBIT operation used in quantum computing, in which a single INPUT maps to two OUTPUTs, the study posited that a genetic analog leveraging T-Pro synthetic transcription factors and cognate genetic elements can be engineered. To accomplish this, first the study constructed two fundamental T-Pro operations: (i) a logical BUFFER operation using the synthetic transcription factor I⁺KSL [9](repressor) mapped to the regulation of red fluorescent protein (RFP) (FIG. 1A), and (ii) an antithetical logical NOT operation via the synthetic transcription factor I^{A(9)}YQR (10,45) (anti-repressor) mapped to the regulation of green fluorescent protein (GFP) (FIG. 1). Notably, both synthetic transcription factors respond to the same INPUT—isopropyl β-D-1-thiogalactopyranoside (IPTG)—with orthogonal DNA binding functions. The study posited that a biological QUBIT logical operation can be designed (FIG. 1C)—and in turn an antithetical PAULI-X operation (FIG. 1D) using similar design rules—in which a 1-INPUT inducible system modulates two disparate OUTPUTs. The study posited that the QUBIT design goal shown in FIG. 1C could be accomplished via three disparate genetic circuits: (i) an inverter-based QUBIT circuit (FIG. 1E), (ii) a partially compressed QUBIT circuit that utilizes an anti-repressor to eliminate the inverter (FIG. 1F), or (iii) a fully compressed QUBIT circuit that leverages an anti-repressor and a bi-directional promoter (FIG. 1G). Prominently, all designs required the use of T-Pro synthetic transcription factors to facilitate single INPUT control over multiple OUTPUTs. The inverter-based QUBIT requires the most resources, 3 RNA polymerases+3 transcription factors. The partially compressed QUBIT requires fewer resources, 2 RNA polymerases+2 transcription factors. Notably, the fully compressed iteration of the QUBIT operation requires the fewest number of resources, 1 RNA polymerase+2 transcription factors. The use of two separate promoters would result in uncoupled transcription of the OUTPUTs due to independent RNA polymerase (RNAp) binding events (FIG. 1F). Accordingly, the study opted for a bi-directional promoter design which yields divergent transcription from a single RNA polymerase binding locus (FIG. 1G), and in principle can impose the smallest metabolic burden on the chassis cell. Although not commonly used in genetic circuits, bidirectional promoters have recently been found to comprise 19% of all detected transcriptional start sites in E. coli, and they also play an important role in gene co-regulation [46]; thus, the design goal given in FIG. 1G is plausible. As designed, the putative genetic QUBIT couples the expression of two OUTPUT states—GFP or RFP production—to the initiation of a single information transfer event.

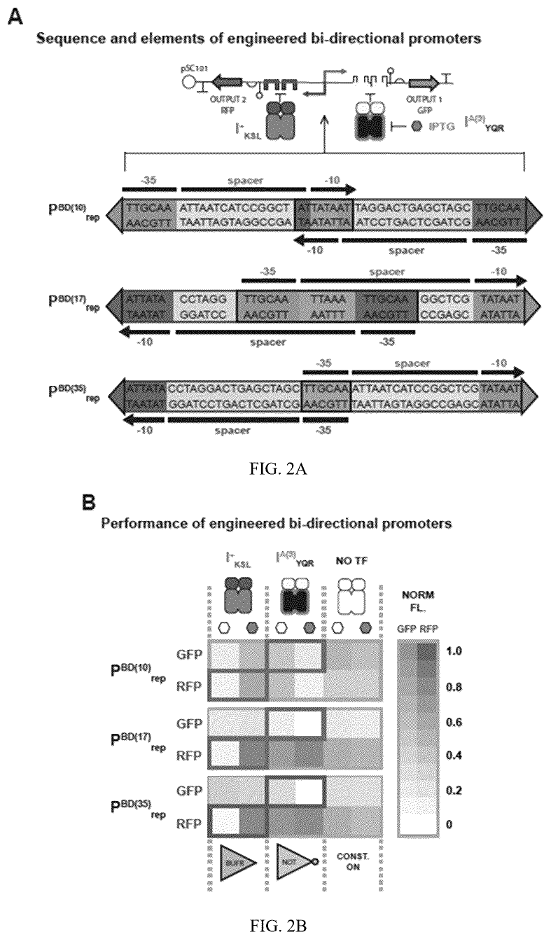

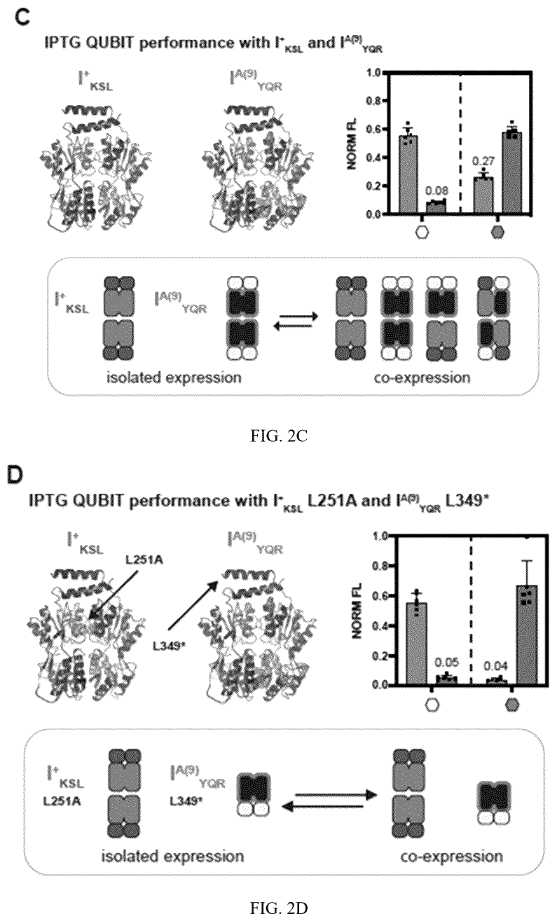

To achieve the fully compressed QUBIT design given in FIG. 1G, three iterations of synthetic bi-directional promoters designed using pTrc and pJ23119 promoter sequences driving GFP and RFP OUTPUTs, respectively (FIG. 2A), were engineered. Namely, the study systematically varied the placement of promoter elements to construct three overlapping and symmetric putative bi-directional promoter configurations: (i) P^{BD(10)}rep—convergent orientation with a single −10 hexamer, (ii) P^{BD(17)}rep—divergent orientation with overlapping spacer regions, and (iii) P^{BD(35)}rep—divergent orientation with a single −35 hexamer. Additionally, a reporter was built with independent unidirectional promoters as a control. To complete the design of the synthetic bi-directional promoters, the study incorporated tandem O^{agg} and O^{sym} synthetic operators, corresponding to cognate synthetic transcription factors I+KSL (repressor) and IA(9)YQR (anti-repressor), to facilitate the discrete regulation of RFP and GFP, respectively. In addition, the study included genetic insulators downstream of each hexamer set to normalize the production of each mRNA transcript (FIG. 2A and FIG. 7).

Next, the study experimentally tested each QUBIT synthetic promoter using single synthetic transcription factors to evaluate discrete INPUT to OUTPUT performance. The study's results indicated that the P^{BD(35)}rep synthetic promoter performed the best in the context of discrete regulation of GFP and RFP (FIG. 2B). The study speculated that the P^{BD(10)}rep configuration exhibited poor regulatory performance due to off-target distal regulation resulting from operator proximity to the −35 hexamers, evidenced by significant changes in expression of the unregulated GFP and RFP channels upon induction. In addition, it was observed that the P^{BD(10)}rep design demonstrated low GFP expression and greater leakiness in RFP regulation compared to P^{BD(35)}rep. To evaluate whether the engineered P^{BD(35)}rep synthetic promoter has the capacity to reduce cellular resource competition (e.g., RNAp [47]) compared to the two independent promoter configuration, an experiment was conducted analogous to the above using a ribose-inducible repressor-anti-repressor pair with the inclusion of an additional plasmid to simulate cellular burden, and measured the change in OUTPUT upon inducer titration. It was observed that for both configurations, the OUTPUT of each unregulated channel (i.e., GFP expression when only R⁺KSL is present, and RFP expression when only R^{A(2)}YQR is present) decreased as the opposing regulated channel was induced, implying a putative cellular resource competition. Moreover, the magnitude of this effect was lower with P^{BD(35)}rep compared to the independent promoter configuration, indicating that P^{BD(35)}rep provided an advantage with regard to mitigating cellular resource competition compared to conventional promoter designs in genetic circuits. Accordingly, the P^{BD(35)}rep synthetic promoter configuration was selected for all logical operations developed in this study.

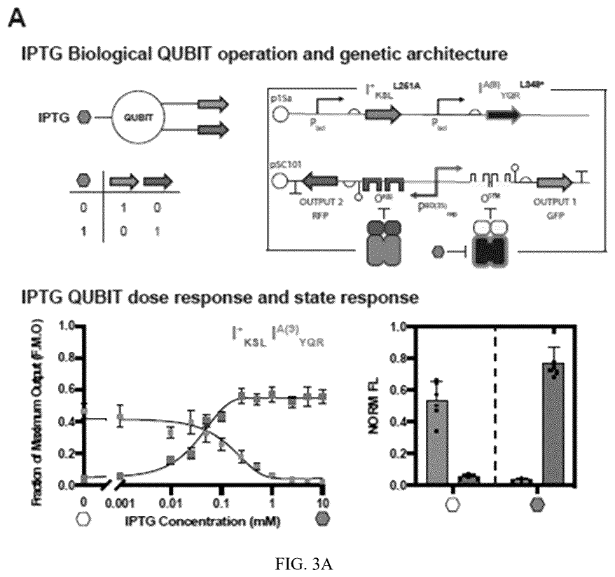

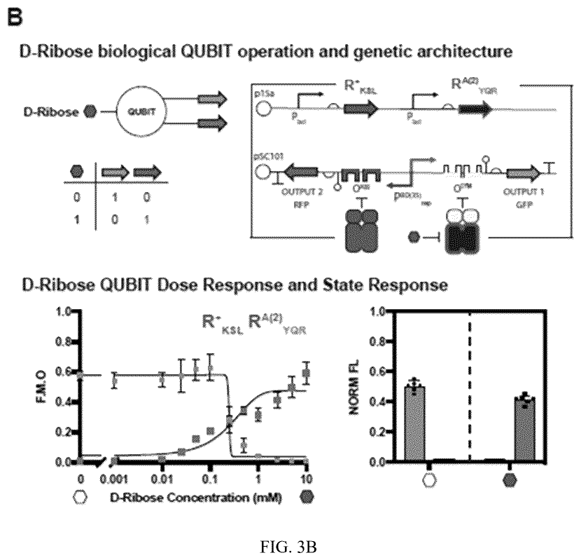

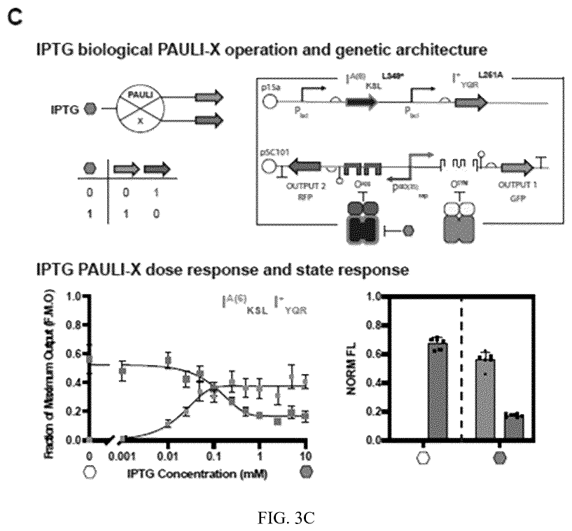

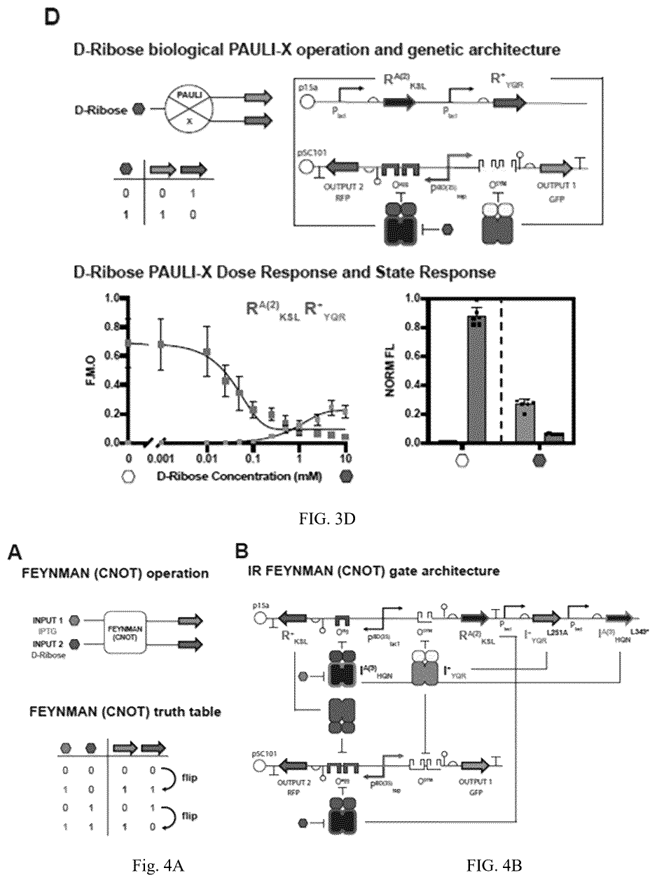

Building and testing a fully compressed biological QUBIT operation. Following the characterization of the discrete INPUT to OUTPUT performance of the P^{BD(35)}rep synthetic promoter, the study tested the said bi-directional promoter in the presence of co-expressed I+KSL (repressor) and IA(9)YQR (anti-repressor), with and without the cognate INPUT (IPTG). Qualitatively, the correct QUBIT phenotype (FIG. 2C) was observed. However, this first iteration QUBIT operation exhibited a significant reduction in regulatory performance relative to the single INPUT biosensor systems—particularly in the GFP anti-repression channel. Given that both synthetic transcription factors function as homotetramers, with homodimers as the fundamental functional unit, the study posited that I⁺KSL and I^{A(9)}YQR monomers formed non-functional heterodimers stabilized by the tetramerization domain (see FIG. 2C inset). To resolve the obfuscated function between the repressor and anti-repressor in the context of the QUBIT operation, the study first introduced an L251A mutation at the monomer-monomer interface of the I⁺KSL repressor. The said mutation in wild-type LacI restricts assembly and function to the tetrameric form of the protein [48]. In addition, 12 residues were truncated from the C-terminus of the I^{A(9)}YQR anti-repressor. This modification in wild-type LacI prevents tetramerization while maintaining the assembly of functional dimers [49]. In principle, the mutated repressor can only function as a tetramer, and the truncated anti-repressor is restricted to dimer formation. The study posited that this set of modifications should eliminate heterodimer and heterotetramer formation—i.e., biasing function to exclusive homogeneous functional assemblies during co-expression (FIG. 2D), resulting in a significant improvement in QUBIT performance. Consistent with the supposition, the study observed a marked increase in performance upon modification of both synthetic transcription factors (FIG. 2D and FIG. 3A).

Next, the study constructed D-ribose-inducible (FIG. 3B) and fructose-inducible biological QUBIT gates, analogous to the IPTG operation described above. Unlike the IPTG system, said operations did not require additional modifications to achieve the desired performance (also see Note 2). In summary, all biological unit operations (i.e., IPTG, D-ribose, and fructose inducible systems) demonstrated logic congruent with QUBIT gates used in quantum computing, where the OUTPUT vector [GFP, RFP] is |0 [symbol] =[1,0] when the INPUT (ligand) is 0, and is |1 [symbol] =[0,1] when the INPUT is 1. Furthermore, each gate exhibited a dose-dependent OUTPUT wherein fractional states of each OUTPUT between the values of 0 and 1 are possible, which is analogous to the properties of QUBITs used in quantum computing. Additionally, the study's quantum-inspired Transcriptional Programming technology can accommodate the regulation of coding and non-coding RNA. To illustrate the latter iteration, the study designed, built, and tested a CRISPRi QUBIT operation. This development expands the utility of this platform technology, and in principle, QUBIT operations can be used with any RNA-based technology that benefits from regulation in the context of a 1 INPUT, 2 OUTPUT operation.

Designing, building, and testing fully compressed biological PAULI-X operations. In quantum information theory, the PAULI-X logical operator performs a bit-flip relative to a QUBIT operation, resulting in an antithetical truth table [50], see relative abstractions given in FIGS. 1C-1D. In other words, the PAULI-X gate is the quantum equivalent of the NOT gate for classical binary computation. The study posited that a biological analogue of the PAULI-X logical operation could be designed using a similar design workflow to that developed for the biological QUBIT operations. The design of a biological PAULI-X logical operation leveraged the P^{BD(35)}rep synthetic promoter developed for the QUBIT operation (see FIG. 2A). Next, the study selected two new synthetic transcription factors that are antithetical in phenotype, while preserving the mapped orthogonal DNA-binding functions, for the set used in the original QUBIT operation. Namely, the study selected (i) the I⁺YQR repressor, opposed to the I^{A(9)}YQR anti-repressor, and (ii) the I^{A(6)}KSL anti-repressor, instead of the I⁺KSL repressor. Both synthetic transcription factors respond to the same INPUT (IPTG), with orthogonal DNA binding functions. In addition, the study carried over the mutations used in the previous section to prevent heterogeneous protein assemblies. In principle, the new set of synthetic T-Pro transcription factors can invert the INPUT to OUTPUT truth table, relative to the corresponding QUBIT operation. Objectively, said remapping of the protein DNA interactions should result in the desired PAULI-X logical operation (see FIG. 3C). The study tested the biological PAULI-X operation, and as expected, the circuit exhibited faithful qualitative performance.

Next, the study developed a second PAULI-X operation responsive to the INPUT D-ribose—see FIG. 3D. As with the previous design, the study used the PBD (35)rep synthetic promoter paired with synthetic transcription factors complementary to those used in the corresponding QUBIT operation, as shown in FIG. 3B. Upon testing the operation, the study's second iteration PAULI-X logic gate resulted in the correct qualitative performance, consistent with the study's general design goal. The ability to confer PAULI-X logic with the study's system demonstrates that the programming structure retains important features of the T-Pro toolkit, which allows for systematic building—i.e., repurposing the synthetic bi-directional promoter—while maintaining circuit compression [10], [13]. In addition, it was posited that QUBIT and PAULI-X operations can be leveraged to systematically engineer advanced biological programs of greater complexity with multi-OUTPUT decision-making.

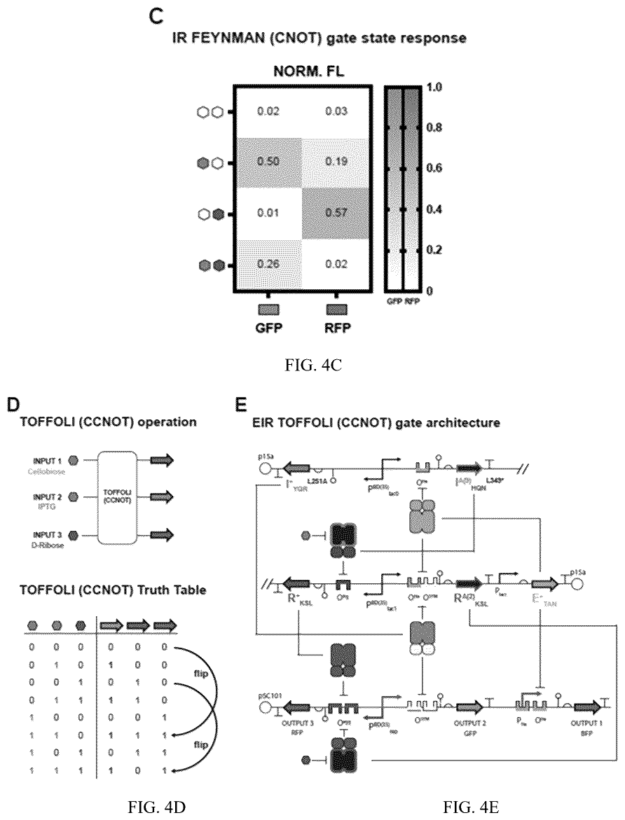

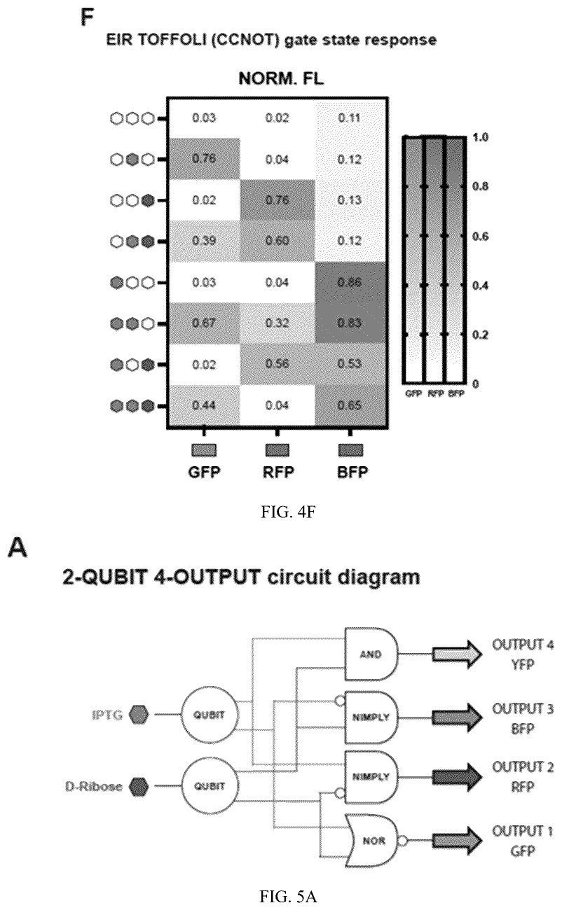

Programming 2-INPUT FEYNMAN (CNOT) logic. In principle, the study can systematically construct advanced reversible logic operations via modularly layering 1-INPUT units—i.e., implementing FEEDFORWARD functions. To demonstrate the ability to construct advanced quantum biocomputing operations via stacking QUBITs, the study designed a FEYNMAN (controlled-NOT, or CNOT) gate, which is the equivalent of a reversible XOR gate, using two-layered QUBIT gates. When combined with QUBITs, Feynman gates are particularly useful in quantum computation as they constitute a universal logic set for simulating any quantum computing circuit. AFEYNMAN gate is a 2-QUBIT operation where the first (control) QUBIT flips the second (target) QUBIT only if the control QUBIT is |1 [symbol] (FIGS. 4A-4B). The study's design started with an IPTG-inducible control QUBIT layer, which regulated R^{A(2)}KSL (anti-repressor) and RKSL (repressor) OUTPUTs—such that (i) an INPUT of 0 maps to R⁺KSL (repressor) OUTPUT, and (ii) an INPUT of 1 maps to R^{A(2)}KSL (anti-repressor) OUTPUT. The D-ribose-inducible OUTPUTs (i.e., synthetic transcription factors) from layer one map to a second QUBIT layer—i.e., the unit operation given in FIG. 3B—such that the terminal layer regulates a set of observable OUTPUTs (see FIG. 4B).

On the first layer, the study engineered a bi-directional promoter analogous to the P^{BD(35)}rep promoter to drive the expression of two transcription factors with a similar level to the individual p^{LacI} promoter while retaining the platform's architectural design. To accomplish this, the study created a small promoter library (1024 variants) on the 17 bp spacer region driving R⁺KSL transcription to reduce promoter secondary structure and tune expression, as well as an RBS/start codon library (64 variants) for the R⁺KSL OUTPUT to optimize circuit performance. Additionally, the study used the phl2 RBS [2] on I^{A(9)}HQN to achieve tighter regulation. Following assembly and phenotypic screening of each library—the study generated a variant which displays qualitative FEYNMAN gate logic (FIG. 4C). Next, the study designed an analogous FEYNMAN gate with the reverse INPUT configuration—i.e., a D-ribose-IPTG inducible system. In this design, the same genetic architecture used in the IPTG-D-ribose system was leveraged, however, the transcription factor regulatory core domains that are responsible for INPUT processing were swapped. This result demonstrated the scalability of the QUBIT programming system in the context of FEEDFORWARD operations, which is congruent with the study's design goal. It was posited that constructing a FEYNMAN gate via alternative strategies would require additional parts (specifically promoters) compared to the design presented in FIG. 4B. To illustrate the aforementioned, a summary of alternative FEYNMAN designs is provided. Briefly, an inverter-based (Cello) design requires 9 inducible promoters, and the apparent FEYNMAN gate designed by Srivastava et al. [42] requires 4 inducible promoters. Whereas the fully compressed design given in FIG. 4B only requires 1 inducible promoter (assuming a bi-directional promoter=1 promoter, which is reasonable given that said promoter can only initiate a single information transfer event). The study posits that this precept can hold for any circuit design that leverages fully compressed QUBIT/PAULI-X operations as fundamental building blocks.

Engineering 3-INPUT TOFFOLI (CCNOT) logic via gate layering. To further demonstrate the platform's layering capability, the study engineered a 3-INPUT, 3-OUTPUT TOFFOLI (controlled-controlled-NOT, or CCNOT) gate by adding a third layer (i.e., an additional control layer) to the IPTG-D-ribose FEYNMAN gate discussed in the previous section. The TOFFOLI gate is a universal reversible logic gate (meaning that any circuit can be constructed from TOFFOLI gates) which inverts the state of a target QUBIT depending on the state of two control QUBITs [51](FIGS. 4D-4E). In the study's design, the constitutively expressed I⁺_{YQR}^{L251A} and I^{A(9)}_{HQN}^{L349*} regulators used in the FEYNMAN gate were added to a third layer, which is regulated by the synthetic cellobiose repressor E⁺TAN. E⁺TAN is expressed constitutively and directly regulates BFP, which was inserted into the reporter downstream of GFP as the control OUTPUT. Additionally, a copy of O^{tla}, the cognate operator to E⁺TAN, was added on the second layer regulating R^{A(2)}KSL to achieve the desired gate operation. Here, cellobiose and IPTG regulated the control layers 1 and 2, respectively, which modulated expression of D-ribose-inducible transcription factors and the state of the target layer (i.e., the combined OUTPUT state) (FIG. 4E). As with the construction of the FEYNMAN gate, the study created and screened independent libraries comprised of 64 variable E⁺TAN and 64 variable R⁺KSL RBS/start codon sequences to optimize regulatory performance. From the study's screen, a variant with the desired TOFFOLI gate performance was identified (FIG. 4F). This result reinforces the study's supposition regarding the modularity and expandability of the quantum logic T-Pro programming structure in the context of FEEDFORWARD operations.

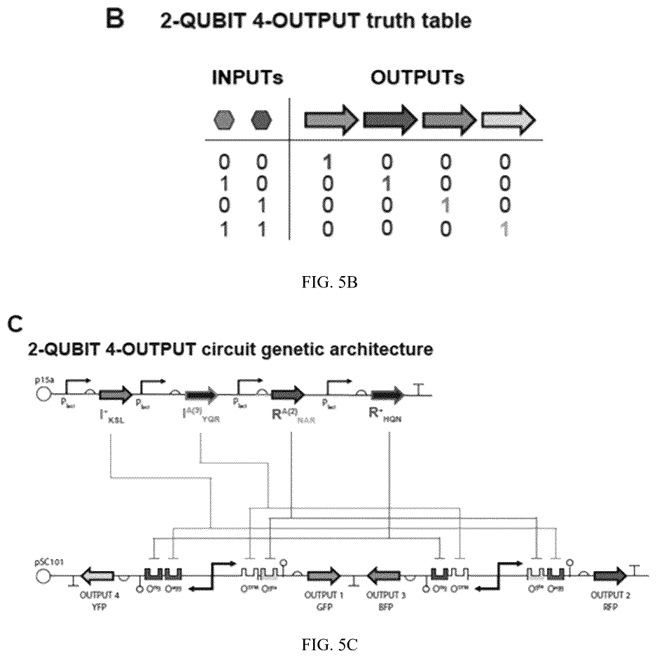

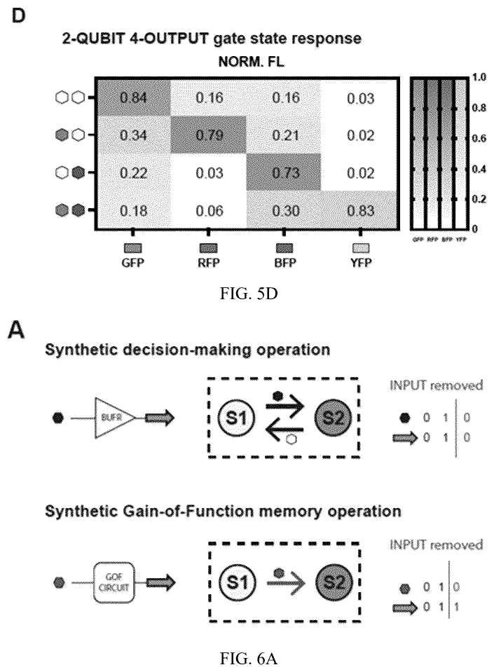

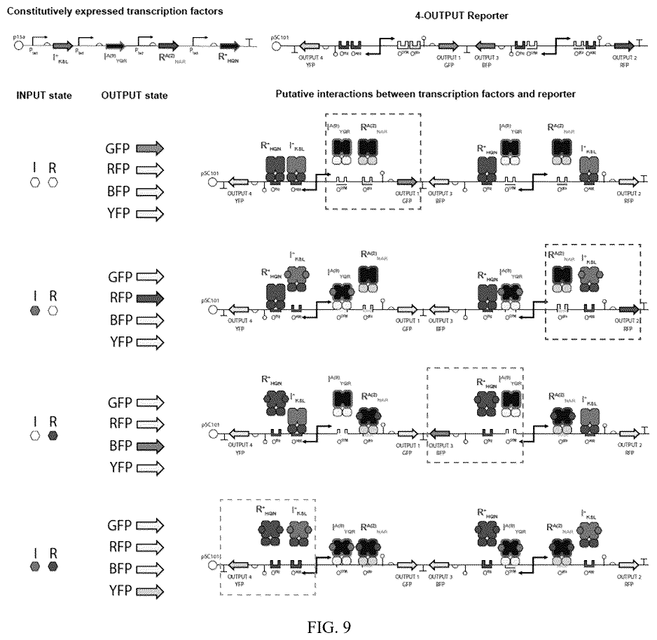

Engineering biological QUBIT logic to modulate 4 OUTPUTs via 2 INPUTs. To demonstrate the utility of the engineered biological QUBIT gates in genetic circuit design, the study built a 4-OUTPUT circuit modulated by two discrete INPUTs—IPTG and D-ribose. This circuit can activate each OUTPUT individually via a unique INPUT permutation, thereby utilizing 100% of the INPUT permutation space (FIGS. 5A-5B). The design is predicated on a single layer containing 2 QUBIT operations, coordinating 2 sets of synthetic transcription factors, such that each set responds to a different INPUT signal—i.e., IPTG or D-ribose (FIG. 5C). Notably, each synthetic transcription factor employs an orthogonal DNA-binding function. The given bi-QUBIT architecture coordinates the expression of 4 disparate OUTPUT reporters via sets of orthogonal tandem operator pairs. To accomplish this, the study re-designed the D-ribose QUBIT to accommodate additional DNA binding functions—i.e., via operators O^{gta} and Ong. Once the study validated the new binding operation, the final bi-QUBIT system was constructed, where QUBIT 1 is mapped to the regulation of YFP or GFP, QUBIT 2 is mapped to regulate BFP or RFP, as shown in the circuit design (FIG. 5C). Finally, the study tested the 2-QUBIT 4-OUTPUT circuit, which was faithful to the design goal—i.e., each INPUT set mapped to a unique OUTPUT—see FIG. 5D and FIG. 9.

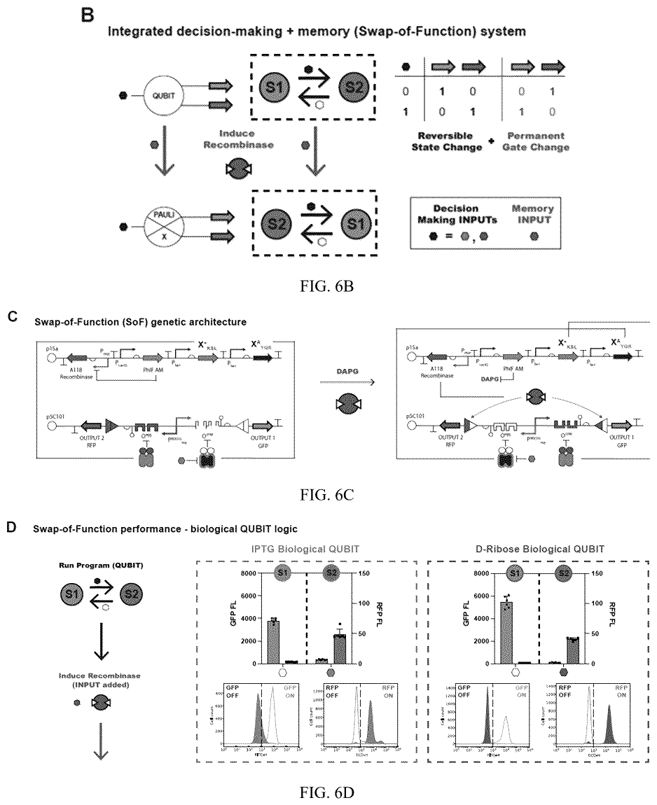

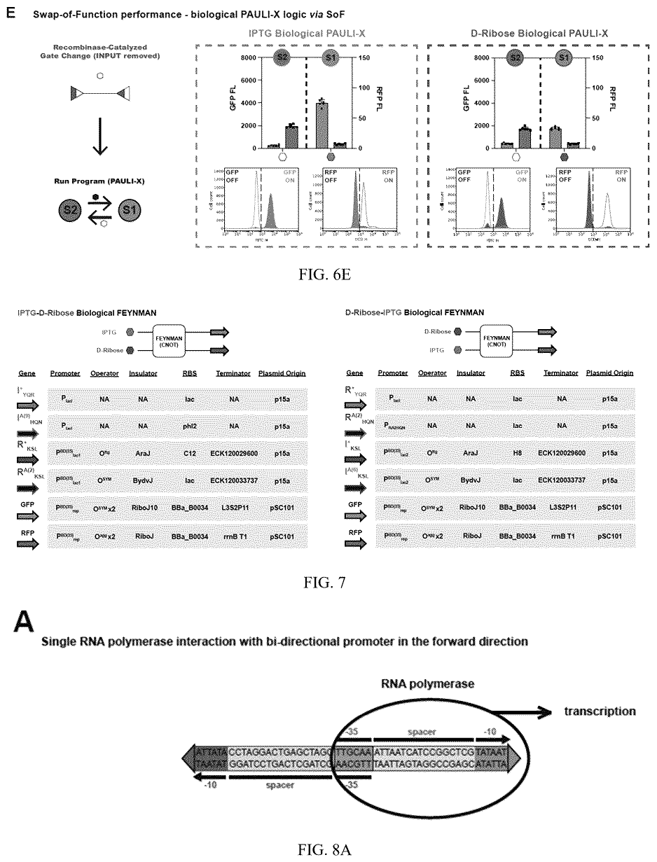

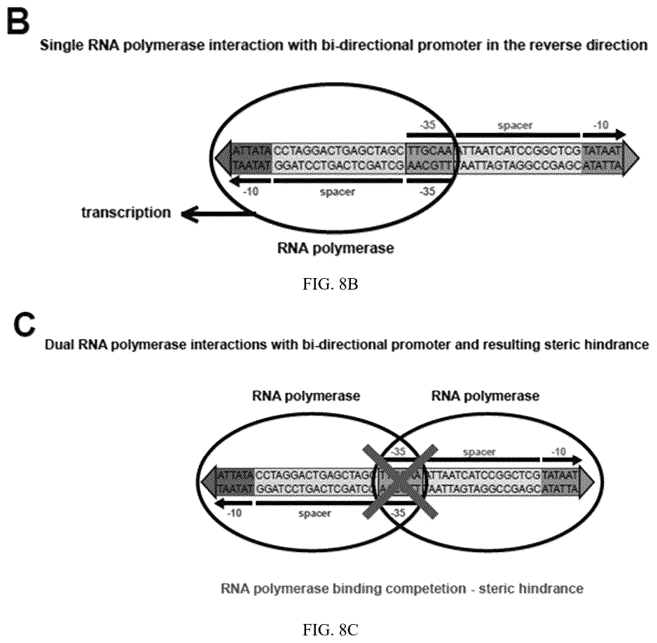

Engineering quantum Swap-of-Function (SoF) memory operations. Genetic memory operations use recombinases to invert or excise genetic elements, which facilitate permanent changes to DNA that are inheritable [26], [52], [53], [54]—see FIG. 6A. A hallmark of synthetic biological intelligence is the integration of decision-making programs with recombinase-based memory operations [43], [44]. Said memory operations can be broadly classified as gain-of-function (GoF) or loss-of-function (LoF) unit operations. The study posited that the bi-directional synthetic promoters and genetic programming structure developed in this work could be leveraged to engineer a third memory operation, in the form of a swap-of-function (SoF), between two fundamental reversible logic operations—FIG. 6B.

The study posited that an invertible synthetic promoter capable of directing two disparate synthetic transcription factors can be engineered, such that said element could be prompted to remap OUTPUT regulation in situ. To accomplish this, the study used the transcription factor PhlF [2], [55] to regulate the serine recombinase A118 [56], which interacts with a set of anti-aligned attachment sites (attB and attP—i.e., oriented to facilitate inversion) flanking the operator sequences of a given bi-directional synthetic promoter (FIG. 6C). In principle, induction of PhlF prompts the expression of recombinase A118, which catalyzes the inversion of the bi-directional promoter and operator sequences. Said recombination event effectively swaps the regulatory control of GFP and RFP OUTPUT channels—i.e., the study can induce the transformation of a biological QUBIT gate to a PAULI-X gate via a Swap-of-Function (SoF) memory operation.

The study built and tested two iterations of the said SoF (QUBIT to PAULI-X) memory operation—(i) one unit operation responsive to IPTG, and (ii) another responsive to D-ribose (FIGS. 6D-6E). In both cases, the SoF memory operation performed as expected, as observed by flow cytometry. This result demonstrates the successful convergence of memory with reversible logic, introducing the ability to engineer intelligent biological systems with expanded functionality and greater dynamic programming capabilities.

Discussion: This study presents a novel toolkit for constructing next-generation synthetic biological decision-making with advanced INPUT economy predicated on reversible logic—i.e., biological QUBIT operations that can be used as building blocks. The modular nature of this technology builds on the study's Transcriptional Programming platform by leveraging previously developed synthetic transcription factors, paired with novel bi-directional synthetic promoters developed in this study. Layering of biological QUBITs allows for the systematic expansion of reversible programs from 1-INPUT, 2-OUTPUT operations to more advanced multiple-INPUT, multiple-OUTPUT reversible logical operations. Commensurate with the tenets of intelligent biological systems, the quantum-inspired technology can be systematically paired with memory operations to form SoF units of inheritable function. Additionally, the use of bi-directional synthetic promoters allows for additional circuit compression—i.e., inherent (and complementary) to the original T-Pro toolkit—via consolidating transcription initiation of two disparate OUTPUTs to a single event.

This quantum-inspired platform for decision-making represents a significant advancement over current reversible gate technology in a single-chassis cell [42]. Namely, the state-of-the-art prior to this study demonstrated and is limited to an apparent FEYNMAN operation, which poses a limit to scalability and biocomputing capability. In contrast, the platform technology developed in this study is based on fundamental QUBIT building blocks that are scalable and resource-efficient (i.e., compressed), enabling unprecedented biocomputing capability and capacity in a single chassis cell. Barring resource limits of the chassis cell, a key limitation of the quantum-inspired wetware is the number of orthogonal synthetic transcription factors available for INPUT processing. The current design space for synthetic transcription factors is on the order of 108, with over 1000 putatively orthogonal INPUTs. In addition, said T-Pro biosensing functions can be expanded via established protein engineering workflows [57]. Accordingly, the study posits that the wetware has the potential of becoming a complete programming language. It is noted that the objective of biocomputing is not to displace abiotic computing—i.e., in silico and the like—rather, the goal is to leverage biocomputing to confer decision-making and synthetic intelligence in biological systems for next-generation biomanufacturing, living therapeutics, and similar biotechnologies.

Taken together, this study represents a paradigm shift in synthetic biology decision-making, enabling the expansion of Transcriptional Programming from Boolean logical operations to quantum-inspired logical operations. The study posits that this platform technology is useful in a myriad of applications that utilize both coding and non-coding RNA, as well as combinations thereof.

Note 1: Quantum Reversibility versus Thermodynamic Reversibility in Transcriptional Programming. Thermodynamic reversibility refers to the ability of a process to run both forward and backward in time without an increase in entropy of the universe. On the other hand, reversibility in the context of computing and quantum logic gates means that the physical implementation of the gate can be reversed without losing information—in other words, this axiom refers to the ability to uniquely determine the INPUT of a computational process from the OUTPUT states. Thus, logical (quantum) reversibility ensures that no information is lost during the computation, meaning each possible OUTPUT corresponds to a unique INPUT. This property is essential in quantum computing, where preserving information throughout the computational process is necessary. Likewise, Transcriptional Programs predicated on quantum reversibility INPUT states can be computed from OUTPUT states—e.g., QUBIT, PAULI-X, TOFFOLI, and the like (see FIGS. 1-4).

NOTE: Quantum computing is not necessarily quantum mechanical; instead, it leverages and emulates an abstraction of reversible logic operations in the form of QUBITs, where a system can represent not just one possibility, but multiple possibilities—commonly referred to as a superposition. In the context of a fundamental QUBIT operation, each INPUT state maps to a unique dual-state OUTPUT—i.e., where the OUTPUT vector is |0 [symbol] =[1,0] when the INPUT is 0, and is |1 [symbol] =[0,1] when the INPUT is 1. QUBITs can be implemented using various components—e.g., trapped ions, photons, atoms, superconducting circuits, defects in diamonds, or any material or system capable of emulating a superposition.

NOTE: QUBIT entanglement refers to a phenomenon where two (or more) QUBITs become linked such that the operations share outcomes. In general, entanglement is a distance-independent event that objectively requires one QUBIT to reliably influence the state of a second QUBIT (or multiple QUBITs concurrently).

Note 2: Characteristics and Features of T-Pro Synthetic Transcription Factors. LacI repressors and anti-repressors in this work contain the native LacI tetramerization domain, which allows for quaternary assembly of dimers into tetramers. In contrast, the RbsR and FruR repressors and anti-repressors used in this work do not contain a tetramerization domain, thereby restricting quaternary formation to dimers. Additionally, the mutations found to confer the LacI anti-repression phenotype by Rondon et al. [45] are different from those found to confer the RbsR and FruR anti-repression phenotypes by Groseclose et al. [10]. Most notably, the L96P mutation in R^{A(2)}YQR, which confers an anti-repression phenotype to the wild-type R+ repressor, is coincidentally positioned at the N-subdomain dimerization interface [10], [61]. The study posits that these properties, along with the absence of a tetramerization domain in both RbsR and FruR anti-repressors, mitigate heterodimerization/tetramerization and support the supposition that the D-ribose and fructose QUBIT gates do not require additional protein modifications.

NOTE: In addition to facilitating circuit compression, the use of T-Pro synthetic transcription factors enables response to two (or more) biosensors to the same INPUT with orthogonal DNA binding functions. This feature is integral to the development of compressed iterations of QUBIT gates. Moreover, said property enables the construction of QUBIT and PAULI-X gates that process the same INPUT that concurrently operate in a single cell.

TABLE 1
Promoters used in this study. Sequences of promoters and promoter sources are
shown. For each promoter, −35 and −10 hexamers are underlined.
For promoters with intercalated operators, the operator
sequence is shown in bold.

| Promoter | DNA Sequence | Source |
|---|---|---|
| placI | GCGCAAAACCTTTCGCGGTATGGCATGAT (SEQ ID NO: 1) | Native |
| | | promoter of |
| | | lacI in E. coli |
| placIQ | GTGCAAAACCTTTCGCGGTATGGCATGAT (SEQ ID NO: 2) | Calos et al. |
| | | [60] |
| PRA2HQN | CGCCAAAACCTTTCGCGGTATGGCATGAT (SEQ ID NO: 3) | This study |
| ptta | TTGACATTTTAAGCGCTTAAAATTATAAT (SEQ ID NO: 4) | Groseclose |
| | | et al. [10] |
| | | Rondon et al. |
| | | [9] |
| PmJ23119 | TGTACAGCTAGCTCAGTCCTAGGTATAAT (SEQ ID NO: 5) | This study |
| PTrc | TTGACAATTAATCATCCGGCTCGTATAATG (SEQ ID NO: 6) | Boer et al. |
| | | [61] |
| PPhlF | TTGACATGATACGAAACGTACCGTATCGT TAAGGT (SEQ ID | Meyer et al. |
| | NO: 7) | [12] |
| PdCas9 | TTGACAGATAGTTGCATCTGTGCTATAAT (SEQ ID NO: 8) | This study |
| PBD(35) | ATTATACCTAGGACTGAGCTAGCTTGCAAATTAATCATCCGGCTC | This study |
| rep | GTATAAT (SEQ ID NO: 9) | |
| PBD(35) | ATCATGCCATACCGCGAAAGGTTTGTACAAACCTTTCGCGGTAT | This study |
| lac0 | GGCATGAT (SEQ ID NO: 10) | |
| PBD(35) | ATCATGCCATATACTGAAAGGTTTGTACAAACCTTTCGCGGTAT | This study |
| lac1 | GGCATGAT (SEQ ID NO: 11) | |
| PBD(35) | ATCATGCCATAAAATTAAAGGTTTGTACAAACCTTTCGCGGTAT | This study |
| lac2 | GGCATGAT (SEQ ID NO: 12) | |

TABLE 2
T-Pro operators used in this study. Sequences of T-Pro operators and
sources are shown. For each operator, base pairs involved
in orthogonal DNA recognition are shown in bold.

| Operator | DNA Sequence | Source |
|---|---|---|
| OSYM | AATTGTGAGCGCTCACAA | Groseclose et al. [10] |
| | TT (SEQ ID NO: 13) | Rondon et al. |
| | | [9] |
| Oagg | AATTAGGAGCGCTCCTAA | Groseclose et al. [10] |
| | TT (SEQ ID NO: 14) | Rondon et al. |
| | | [9] |
| Ottg | AATTTTGAGCGCTCAAAA | Groseclose et al. [10] |
| | TT (SEQ ID NO: 15) | Rondon et al. |
| | | [9] |
| Otta | AATTTTAAGCGCTTAAAAT | Groseclose et al. [10] |
| | T (SEQ ID NO: 16) | Rondon et al. |
| | | [9] |
| Ogta | AATTGTAAGCGCTTACAA | Groseclose et al. [10] |
| | TT (SEQ ID NO: 17) | Rondon et al. [9] |

TABLE 3
RBSs used in this study. Sequences of RBSs and their sources are shown.

| RBS | DNA Sequence | Source |
|---|---|---|
| lac | GGAAGAGAGTCAATTCAGGGTGGTGAAT (SEQ ID | RBS from |
| | NO: 18) | E. coli |
| | | genome |
| BBa_ | AAAGAGGAGAAA (SEQ ID NO: 19) | Elowitz et al. [62] |
| B0034 | | |
| C12 | GCTGGGAAGGAATACGTAAGAATTA (SEQ ID NO: 20) | This study |
| ph12 | GGAAGAGAGTCAATTCATGGGGGTGAAT (SEQ ID | Meyer et al. |
| | NO: 21) | [12] |
| H8 | GGAAGAGAGTCAATTCATGGGGGTGAAT (SEQ ID | This study |
| | NO: 22) | |
| E21 | GCTGGGAAGGAATCCGTATGAATTAGCTGGGAAGG | This study |
| | AATGCGTAAGAATTA (SEQ ID NO: 23) | |
| F9 | CCTGGGAAGGAATGCGTAGGAATTA (SEQ ID NO: 24) | This study |
| a118 | CAACCATCGTATTGAAGGTGGCTAAC (SEQ ID NO: | This study |
| | 25) | |
| prgJ | CTTTAATTTAACGTAAATAAGGAAGTCATT (SEQ ID | Salis et. al |
| | NO: 26) | [63] |
| eng1 | AACAAAATGAGGAGGTACTGAG (SEQ ID NO: 27) | Salis et. al |
| | | [63] |
| eng3 | ACAACTTAAGGAGGTATTC (SEQ ID NO: 28) | Salis et. al |
| | | [63] |
| eng5 | CTGGTGAGGAGGTGAGA (SEQ ID NO: 29) | Salis et. al |
| | | [63] |
| eng6 | AAAGTAAGGAGGCGCGGCT (SEQ ID NO: 30) | Salis et. al |
| | | [63] |

TABLE 4
Genetic insulators used in this study. DNA sequences of insulators are shown.

| Insulator | DNA Sequence | Source |
|---|---|---|
| RiboJ | AGCTGTCACCGGATGTGCTTTCCGGTCTGATGAGTCCGT | Lou et al. [64] |
| | GAGGAC GAAACAGCCTCTACAAATAATTTTGTTTAA | Nielsen et al. |
| | (SEQ ID NO: 31) | [5] |
| RiboJ10 | AGCGCTCAACGGGTGTGCTTCCCGTTCTGATGAGTCCG | Nielsen et al. [5] |
| | TGAGGA CGAAAGCGCCTCTACAAATAATTTTGTTTAA | |
| | (SEQ ID NO: 32) | |
| AraJ | AGTGGTCGTGATCTGAAACTCGATCACCTGATGAGCTC | Dufour et al. [65] |
| | AAGGCAGAGCGAAACCACCTCTACAAATAATTTTGTTTA | Nielsen et al. [5] |
| | A (SEQ ID NO: 33) | |
| BydvJ | AGGGTGTCTCAAGGTGCGTACCTTGACTGATGAGTCCG | Khvorova et al. [66] |
| | AAAGGACGAAACACCCCTCTACAAATAATTTTGTTTAA | Nielsen et al. [5] |
| | (SEQ ID NO: 34) | |
| ElvJ | AGCCCCATAGGGTGGTGTGTACCACCCCTGATGAGTCC | Dufour et al. [65] |
| | AAAAGGACGAAATGGGGCCTCTACAAATAATTTTGTTTA | Nielsen et al. [5] |
| | A (SEQ ID NO: 35) | |
| PlmJ | AGTCATAAGTCTGGGCTAAGCCCACTGATGAGTCGCTG | Lou et al. [64] |
| | AAATGCGACGAAACTTATGACCTCTACAAATAATTTTGT | Nielsen et al. [5] |
| | TTAA (SEQ ID NO: 36) | |
| CchJ | AGTTCCAGTCGAGACCTGAAGTGGGTTTCCTGATGAGG | Khvorova et al. [66] |
| | CTGTGGAGAGAGCGAAAGCTTTACTCCCGCACAAGCCG | Nielsen et al. [5] |
| | AAACTGGAACCTCTACAAATAATTTTGTTTAA (SEQ ID | |
| | NO: 37) | |
| HH1 | CATGAGCTGATGAGTCCGTGAGGACGAAACGAGTAAGC | Pley et al. [67] |
| | TCGTC (SEQ ID NO: 38) | Gao et al. |
| | | [68] |
| HH2 | GCAGTAAGCTGCCTGAAGAGCTGAGTGCAGCGAAAAA | Pley et al. [67] |
| | AA (SEQ ID NO: 39) | Gao et al. [68] This |
| | | study |
| HH3 | GTACCACTGATGAGTCCGTGAGGACGAAACGAGTAAGC | Pley et al. [67] |
| | TCGTC (SEQ ID NO: 40) | Gao et al. |
| | | [68] |
| HDV | GGCCGGCATGGTCCCAGCCTCCTCGCTGGCGCCGGCTG | Ferre-D'Amare et |
| | GGCAAC ATGCTTCGGCATGGCGAATGGGAC (SEQ ID | al. |
| | NO: 41) | [69] |
| | | Gao et al. [68] |

TABLE 5
Transcriptional terminators used in this study. DNA sequences
and sources of transcriptional terminators are shown.

| Terminator | DNA Sequence | Source |
|---|---|---|
| rrnB T1 | CAAATAAAACGAAAGGCTCAGTCGAAAGACTGGGCCTTTC | Terminator |
| | GTTTTAT CTGTTGTTTGTCGGTGAACGCTCTC | from rrnB |
| | (SEQ ID NO: 42) | gene in |
| | | E. coli |
| L3S2P11 | CTCGGTACCAAATTCCAGAAAAGAGACGCTTTCGAGCGTC | Chen et al. |
| | TTTTTTCGTTTTGGTCC (SEQ ID NO: 43) | [70] |
| ECK120029600 | TTCAGCCAAAAAACTTAAGACCGCCGGTCTTGTCCACTACC | Chen et al. |
| | TTGCAGTAATGCGGTGGACAGGATCGGCGGTTTTCTTTTCT | [70] |
| | CTTCTCAA (SEQ ID NO: 44) | |
| ECK120033737 | GGAAACACAGAAAAAAGCCCGCACCTGACAGTGCGGGCT | Chen et al. |
| | TTTTTTTTCGACCAAAGG (SEQ ID NO: 45) | [70] |
| ECK120033736 | AACGCATGAGAAAGCCCCCGGAAGATCACCTTCCGGGGGC | Chen et al. |
| | TTTTTTA TTGCGC (SEQ ID NO: 46) | [70] |
| ECK120010818 | GTCAGTTTCACCTGTTTTACGTAAAAACCCGCTTCGGCGGG | Chen et al. |
| | TTTTTACTTTTGG (SEQ ID NO: 47) | [70] |

TABLE 6
Coding and (non-coding) sgRNA sequences used in this study.
DNA sequences are shown.

| Coding Sequence | DNA Sequence |
|---|---|
| sfGFP (GFP) | ATGAGCAAAGGAGAAGAACTTTTCACTGGAGTTGTCCC |
| | AATTCTTGTTGAATTAGATGGTGATGTTAATGGGCACAA |
| | ATTTTCTGTCCGTGGAGAGGGTGAAGGTGATGCTACAA |
| | ACGGAAAGCTTACCCTTAAATTTATTTGCACTACTGGAA |
| | AACTACCTGTTCCATGGCCAACACTTGTCACTACTCTCA |
| | CTTATGGTGTTCAATGCTTTTCCCGTTATCCGGATCATAT |
| | GAAACGGCATGACTTTTTCAAGAGTGCCATGCCCGAAG |
| | GTTATGTACAGGAACGCACTATATCTTTCAAAGATGACG |
| | GGACTTACAAGACGCGTGCTGAAGTCAAGTTTGAAGGT |
| | GATACCCTTGTTAATCGTATCGAGTTAAAAGGTATTGATT |
| | TTAAAGAAGATGGAAACATTCTCGGACACAAACTTGAG |
| | TACAACTTTAACTCACACAATGTATACATCACGGCAGAC |
| | AAACAAAAGAATGGAATCAAAGCTAACTTCAAAATTCG |
| | CCACAACGTTGAAGATGGATCCGTTCAACTAGCAGACC |
| | ATTATCAACAAAATACTCCAATTGGCGATGGCCCTGTCC |
| | TTTTACCAGACAACCATTACCTGTCGACACAATCTGTCC |
| | TTTCGAAAGATCCCAACGAAAAGCGTGACCACATGGTC |
| | CTTCTTGAGTTTGTAACTGCTGCTGGGATTACACATGGC |
| | ATGGATGAGCTCTACAAA (SEQ ID NO: 48) |
| mCherry (RFP) | ATGGTTAGCAAGGGCGAGGAAGACAACATGGCGATCAT |
| | CAAAGAGTTCATGCGTTTTAAGGTTCACATGGAAGGTTC |
| | TGTTAACGGTCACGAGTTCGAAATCGAAGGTGAAGGCG |
| | AAGGCCGTCCGTACGAAGGTACCCAGACCGCGAAACTC |
| | AAGGTTACCAAAGGTGGTCCGCTGCCGTTCGCGTGGGA |
| | CATCCTCTCTCCGCAATTCATGTACGGTTCTAAAGCGTA |
| | CGTTAAACATCCGGCAGATATCCCGGACTACCTGAAACT |
| | GTCTTTCCCGGAAGGCTTCAAATGGGAACGTGTTATGA |
| | ACTTTGAAGACGGCGGTGTTGTTACCGTTACCCAGGAC |
| | TCTTCTCTGCAGGACGGTGAATTTATCTATAAGGTTAAA |
| | CTCCGTGGTACTAACTTCCCGTCTGACGGTCCGGTTATG |
| | CAGAAAAAGACCATGGGTTGGGAAGCGTCTTCTGAACG |
| | TATGTACCCGGAGGATGGTGCGCTGAAAGGCGAGATCA |
| | AACAGCGTCTCAAACTGAAAGACGGTGGTCACTACGAC |
| | GCGGAAGTAAAAACCACCTACAAAGCTAAAAAGCCAG |
| | TTCAGCTGCCGGGTGCGTACAACGTGAACATTAAACTG |
| | GACATCACCTCTCACAACGAAGACTACACCATTGTTGA |
| | ACAGTACGAACGTGCGGAGGGTCGTCACTCTACCGGTG |
| | GTATGGACGAACTGTACAAATAA (SEQ ID NO: 49) |
| tagBFP2 (BFP) | ATGAGCGAGCTGATTAAGGAGAACATGCACATGAAGCT |
| | GTACATGGAGGGCACCGTGGACAACCATCACTTCAAGT |
| | GCACATCCGAGGGCGAAGGCAAGCCCTACGAGGGCAC |
| | CCAGACCATGAGAATCAAGGTGGTCGAGGGCGGCCCTC |
| | TCCCCTTCGCCTTCGACATCCTGGCTACTAGCTTCCTCTA |
| | CGGCAGCAAGACCTTCATCAACCACACCCAGGGCATCC |
| | CCGACTTCTTCAAGCAGTCCTTCCCTGAGGGCTTCACAT |
| | GGGAGAGAGTCACCACATACGAAGACGGGGGCGTGCT |
| | GACCGCTACCCAGGACACCAGCCTCCAGGACGGCTGCC |
| | TCATCTACAACGTCAAGATCAGAGGGGTGAACTTCACA |
| | TCCAACGGCCCTGTGATGCAGAAGAAAACACTCGGCTG |
| | GGAGGCCTTCACCGAGACGCTGTACCCCGCTGACGGCG |
| | GCCTGGAAGGCAGAAACGACATGGCCCTGAAGCTCGT |
| | GGGCGGGAGCCATCTGATCGCAAACATCAAGACCACAT |
| | ATAGATCCAAGAAACCCGCTAAGAACCTCAAGATGCCT |
| | GGCGTCTACTATGTGGACTACAGACTGGAAAGAATCAA |
| | GGAGGCCAACAACGAGACCTACGTCGAGCAGCACGAG |
| | GTGGCAGTGGCCAGATACTGCGACCTCCCTAGCAAACT |
| | GGGGCACTAA (SEQ ID NO: 50) |
| phi YFP (YFP) | ATGAGCTCCGGCGCGCTGCTGTTTCACGGTAAGATCCCG |
| | TACGTGGTTGAGATGGAGGGCAACGTTGACGGTCATAC |
| | CTTCTCCATCCGTGGTAAAGGTTATGGCGACGCTAGCGT |
| | AGGTAAAGTGGACGCGCAGTTCATCTGCACTACTGGTG |
| | ACGTGCCGGTTCCGTGGTCTACTCTGGTGACTACCCTGA |
| | CCTATGGTGCCCAGTGCTTCGCCAAATACGGTCCAGAAC |
| | TGAAAGATTTCTACAAAAGCTGCATGCCTGAAGGCTATG |
| | TTCAGGAACGTACCATTACCTTCGAAGGCGACGGTGTG |
| | TTCAAAACCCGCGCGGAAGTAACCTTTGAAAACGGCTC |
| | CGTTTACAACCGCGTGAAACTGAACGGTCAAGGCTTCA |
| | AAAAAGACGGTCACGTGCTGGGCAAGAACCTGGAATT |
| | CAACTTTACCCCGCACTGCCTGTACATCTGGGGTGACCA |
| | GGCGAACCATGGCCTGAAATCTGCGTTCAAGATCATGC |
| | ACGAGATCACTGGCTCTAAGGAAGACTTTATCGTGGCC |
| | GACCACACTCAGATGAACACGCCAATTGGCGGTGGTCC |
| | GGTTCACGTGCCGGAATATCACCACATCACGTACCACGT |
| | TACCCTGTCCAAAGACGTGACTGATCACCGTGACAACA |
| | TGTCTCTGGTTGAAACTGTTCGTGCGGTGGATTGCCGTA |
| | AAACGTACCTGTAA (SEQ ID NO: 51) |
| I⁺YQR | ATGAAACCGGTAACGCTTTACGACGTAGCCGAATACGC |
| Note: The DNA- binding | CGGGGTATCCTACCAGACCGTGAGTCGTGTCGTAAATCA |
| domain YQR is swapped by | GGCCTCCCATGTAAGCGCTAAGACAAGAGAGAAAGTGG |
| mutating positions Y17, Q18, | AAGCGGCTATGGCAGAGTTGAATTACATCCCCAACCGT |
| and R22 to confer alternate | GTCGCCCAACAGCTAGCGGGTAAACAGTCATTACTTATC |
| DNA-binding | GGCGTTGCGACAAGCTCTTTGGCGTTACACGCCCCCAG |
| (e.g., I⁺KSL would be Y17K, | TCAGATAGTTGCAGCCATCAAGAGTAGGGCTGACCAGC |
| Q18S, and R22L) | TAGGCGCAAGTGTCGTAGTGTCAATGGTTGAGAGATCA |
| | GGGGTCGAGGCTTGTAAGACGGCTGTGCATAACTTACTT |
| | GCCCAAAGAGTCTCTGGTTTAATAATAAATTACCCATTG |
| | GATGACCAAGATGCGATAGCGGTTGAGGCAGCTTGCAC |
| | AAATGTCCCCGCCCTTTTCTTAGACGTGTCCGATCAAAC |
| | GCCGATCAACTCCATTATCTTCTCTCACGAGGACGGCAC |
| | TAGATTAGGCGTGGAACATCTTGTAGCGCTGGGGCACC |
| | AGCAAATCGCATTGCTAGCGGGACCGCTATCTTCCGTTT |
| | CAGCTCGTCTAAGACTTGCGGGTTGGCATAAGTATTTGA |
| | CAAGAAATCAAATTCAGCCGATAGCCGAGCGTGAGGGG |
| | GATTGGTCAGCAATGTCAGGTTTCCAGCAGACGATGCA |
| | GATGCTGAACGAAGGGATTGTCCCAACTGCCATGCTTG |
| | TCGCTAACGACCAGATGGCGCTGGGCGCCATGCGTGCT |
| | ATAACCGAGTCAGGCTTAAGGGTGGGCGCGGACATCTC |
| | AGTTGTGGGATACGACGATACAGAGGATTCATCCTGTTA |
| | CATACCGCCTTTGACCACGATTAAGCAAGACTTTCGTCT |
| | ATTAGGACAGACGTCAGTAGACAGGTTATTGCAGCTGA |
| | GTCAGGGACAGGCAGTCAAAGGTAACCAGCTTCTACCT |
| | GTCTCTCTAGTCAAGAGAAAGACGACCCTGGCTCCGAA |
| | TACGCAGACAGCCTCACCGCGTGCTCTTGCGGACTCAC |
| | TTATGCAGCTGGCCAGGCAGGTTTCCAGACTAGAAAGC |
| | GGACAATGA (SEQ ID NO: 52) |
| I⁺YQR L241A | ATGAAACCGGTAACGCTTTACGACGTAGCCGAATACGC |
| Note: The DNA-binding | CGGGGTATCCTACCAGACCGTGAGTCGTGTCGTAAATCA |
| domain YQR is swapped by | GGCCTCCCATGTAAGCGCTAAGACAAGAGAGAAAGTGG |
| mutating positions Y17, Q18, | AAGCGGCTATGGCAGAGTTGAATTACATCCCCAACCGT |
| and R22 to confer alternate | GTCGCCCAACAGCTAGCGGGTAAACAGTCATTACTTATC |
| DNA-binding | GGCGTTGCGACAAGCTCTTTGGCGTTACACGCCCCCAG |
| (e.g., I⁺KSL would be Y17K, | TCAGATAGTTGCAGCCATCAAGAGTAGGGCTGACCAGC |
| Q18S, and R22L) | TAGGCGCAAGTGTCGTAGTGTCAATGGTTGAGAGATCA |
| | GGGGTCGAGGCTTGTAAGACGGCTGTGCATAACTTACTT |
| | GCCCAAAGAGTCTCTGGTTTAATAATAAATTACCCATTG |
| | GATGACCAAGATGCGATAGCGGTTGAGGCAGCTTGCAC |
| | AAATGTCCCCGCCCTTTTCTTAGACGTGTCCGATCAAAC |
| | GCCGATCAACTCCATTATCTTCTCTCACGAGGACGGCAC |
| | TAGATTAGGCGTGGAACATCTTGTAGCGCTGGGGCACC |
| | AGCAAATCGCATTGCTAGCGGGACCGCTATCTTCCGTTT |
| | CAGCTCGTCTAAGACTTGCGGGTTGGCATAAGTATTTGA |
| | CAAGAAATCAAATTCAGCCGATAGCCGAGCGTGAGGGG |
| | GATTGGTCAGCAATGTCAGGTTTCCAGCAGACGATGCA |
| | GATGCTGAACGAAGGGATTGTCCCAACTGCCATGCTTG |
| | TCGCTAACGACCAGATGGCGGCGGGCGCCATGCGTGCT |
| | ATAACCGAGTCAGGCTTAAGGGTGGGCGCGGACATCTC |
| | AGTTGTGGGATACGACGATACAGAGGATTCATCCTGTTA |
| | CATACCGCCTTTGACCACGATTAAGCAAGACTTTCGTCT |
| | ATTAGGACAGACGTCAGTAGACAGGTTATTGCAGCTGA |
| | GTCAGGGACAGGCAGTCAAAGGTAACCAGCTTCTACCT |
| | GTCTCTCTAGTCAAGAGAAAGACGACCCTGGCTCCGAA |
| | TACGCAGACAGCCTCACCGCGTGCTCTTGCGGACTCAC |
| | TTATGCAGCTGGCCAGGCAGGTTTCCAGACTAGAAAGC |
| | GGACAATGA (SEQ ID NO: 53) |
| IA(9)YQR | GTGAAACCAGTAACGTTATACGATGTCGCAGAGTATGCC |
| Note: The DNA-binding | GGTGTCTCTTATCAGACCGTTTCCCGCGTGGTGAACCAG |
| domain YQR is swapped by | GCCAGCCACGTTTCTGCGAAAACGCGGGAAAAAGTGG |
| mutating positions Y17, Q18, | AAGCGGCGATGGCGGAGCTGAATTACATTCCCAACCGC |
| and R22 to confer alternate | GTGGCACAACAACTGGCGGGCAAACAGTCGTTGCTGAT |
| DNA-binding | TGGCGTTGCCACCTCCAGTCTGGCCCTGCACGCGCTGT |
| (e.g., JA(9)KSL would be Y17K, | CGCAAATTGTCGCGGCGATTAAATCTCGCGCCTATCAAC |
| Q18S, and R22L) | TGGGTGCCAGCGTGTTCGTGTCGATGGTAGAACGAAGC |
| | GGCATCGAAGCCTGTAAAACGGCGGTGCACAATCTTCT |
| | CGCGCAACGCGTCAGTGGGCTGATCATTAACTATCCGCT |
| | GGATAACCAGGATGCCATTGCTGTGGAAGCTGCCTGCA |
| | CTAATGTTCCGGCGTTATTTCTTGATGTCTCTGACCAGAC |
| | ACCCATCAACAGTATTATTTTCTCCCATGAAGACGGTAC |
| | GCGACTGGGCGTGGAGCATCTGGTCGCATTGGGTCACC |
| | AGCAAATCGCGCTGTTAGCGGGCCCATTAAGTTCTGTCT |
| | CGGCGCGTCTGCGTCTGGCTGGCTGGCATAAATATCTCA |
| | CTCGCAATCAAATTCAGCCGATAGCGGAACGGGAAGGC |
| | GACTGGAGTGCCATGTCCGGTTTTCAACAAACCATGCA |
| | AATGCTGAATGAGGGCATCGTTCCCACTGCGATGCTGGT |
| | TGCCAACGATCAGATGGCGCTGGGCGCAATGCGCGCCA |
| | TTACCGAGACCGGGCTGCGCGTTGGTGCGGATATCTCG |
| | GTAGTGGGATACGACGATACCGAAGACAGCTCATGTTAT |
| | ATCCCGCCGTTAACCACCATCAAACAGGATTTTCGCCTG |
| | CTGGGGCAAACCAGCGTGGACCGCTTGCTGCAACTCTC |
| | TCAGGGCCAGGCGGTGAAGGGCAATCAGCTGTTGCCCG |
| | TCTCACTGGTGAAAAGAAAAACCACCCTGGCGCCCAAT |
| | ACGCAAACCGCCTCTCCCCGCGCGTTGGCCGATTCATTA |
| | ATGCAGCTGGCACGACAGGTTTCCCGACTGGAAAGCGG |
| | GCAGTGA (SEQ ID NO: 54) |
| IA(9)YQR L349* | ATGAAACCAGTAACGTTATACGATGTCGCAGAGTATGCC |
| Note: The DNA- binding | GGTGTCTCTTATCAGACCGTTTCCCGCGTGGTGAACCAG |
| domain YQR is swapped by | GCCAGCCACGTTTCTGCGAAAACGCGGGAAAAAGTGG |
| mutating positions Y17, Q18, | AAGCGGCGATGGCGGAGCTGAATTACATTCCCAACCGC |
| and R22 to confer alternate | GTGGCACAACAACTGGCGGGCAAACAGTCGTTGCTGAT |
| DNA-binding | TGGCGTTGCCACCTCCAGTCTGGCCCTGCACGCGCTGT |
| (e.g., IA(9)KSL would be Y17K, | CGCAAATTGTCGCGGCGATTAAATCTCGCGCCTATCAAC |
| Q18S, | TGGGTGCCAGCGTGTTCGTGTCGATGGTAGAACGAAGC |
| and R22L) | GGCATCGAAGCCTGTAAAACGGCGGTGCACAATCTTCT |
| | CGCGCAACGCGTCAGTGGGCTGATCATTAACTATCCGCT |
| | GGATAACCAGGATGCCATTGCTGTGGAAGCTGCCTGCA |
| | CTAATGTTCCGGCGTTATTTCTTGATGTCTCTGACCAGAC |
| | ACCCATCAACAGTATTATTTTCTCCCATGAAGACGGTAC |
| | GCGACTGGGCGTGGAGCATCTGGTCGCATTGGGTCACC |
| | AGCAAATCGCGCTGTTAGCGGGCCCATTAAGTTCTGTCT |
| | CGGCGCGTCTGCGTCTGGCTGGCTGGCATAAATATCTCA |
| | CTCGCAATCAAATTCAGCCGATAGCGGAACGGGAAGGC |
| | GACTGGAGTGCCATGTCCGGTTTTCAACAAACCATGCA |
| | AATGCTGAATGAGGGCATCGTTCCCACTGCGATGCTGGT |
| | TGCCAACGATCAGATGGCGCTGGGCGCAATGCGCGCCA |
| | TTACCGAGACCGGGCTGCGCGTTGGTGCGGATATCTCG |
| | GTAGTGGGATACGACGATACCGAAGACAGCTCATGTTAT |
| | ATCCCGCCGTTAACCACCATCAAACAGGATTTTCGCCTG |
| | CTGGGGCAAACCAGCGTGGACCGCTTGCTGCAACTCTC |
| | TCAGGGCCAGGCGGTGAAGGGCAATCAGCTGTTGCCaG |
| | TaTCACTGGTGAAAAGAAAAACCACCCTGGCGCCCAAT |
| | ACGCAAACCGCCTCTCCCCGCGCGTTGGCCGATTCATTA |
| | ATGCAGTAA (SEQ ID NO: 55) |
| R⁺YQR | ATGAAACCGGTAACTTTGTACGACGTAGCTGAATACGCT |
| Note: The DNA-binding | GGTGTATCTTACCAGACTGTATCTCGTGTAGTAAACCAG |
| domain YQR is swapped by | GCTTCTCACGTATCTGCTAAAACTCGTGAAAAAGTAGA |
| mutating positions Y17, Q18, | AGCTGCTATGGCTGAATTGAACTACATCCCGAACCGTGT |
| and R22 to confer alternate | AGCTCAGCAGTTGGCTGGTAAAGCTTCTCACACTATCG |
| DNA-binding | GTATGTTGATCACTGCTTCTACTAACCCGTTCTACTCTGA |
| (e.g., R⁺KSL would be Y17K, | ATTGGTACGTGGTGTAGAACGTTCTTGTTTCGAACGTGG |
| Q18S, | TTACTCTTTGGTATTGTGTAACACTGAAGGTGACGAACA |
| and R22L) | GCGTATGAACCGTAACTTGGAAACTTTGATGCAGAAAC |
| | GTGTAGACGGTTTGTTGTTGTTGTGTACTGAAACTCACC |
| | AGCCGTCCCGTGAAATCATGCAGCGTTACCCGACTGTAC |
| | CGACTGTAATGATGGACTGGGCTCCGTTCGACGGTGAC |
| | TCTGACTTGATCCAGGACAACTCTTTGTTGGGTGGTGAC |
| | TTGGCTACTCAGTACTTGATCGACAAAGGTCACACTCGT |
| | ATCGCTTGTATCACTGGTCCGTTGGACAAAACTCCGGCT |
| | CGTTTGCGTTTGGAAGGTTACCGTGCTGCTATGAAACGT |
| | GCTGGTTTGAACATCCCGGACGGTTACGAAGTAACTGG |
| | TGACTTCGAATTCAACGGTGGTTTCGACGCTATGCGTCA |
| | GTTGTTGTCTCACCCGTTGCGTCCGCAGGCTGTATTCAC |
| | TGGTAACGACGCTATGGCTGTAGGTGTATACCAGGCTTT |
| | GTACCAGGCTGAATTGCAGGTACCGCAGGACATCGCTG |
| | TAATCGGTTACGACGACATCGAATTGGCTTCTTTCATGA |
| | CTCCGCCGTTGACTACTATCCACCAGCCGAAAGACGAA |
| | TTGGGTGAATTGGCTATCGACGTATTGATCCACCGTATCA |
| | CTCAGCCGACTTTGCAGCAGCAGCGTTTGCAGTTGACT |
| | CCGATCTTGATGGAACGTGGTTCTGCTTAA (SEQ ID NO: |
| | 56) |
| RA(2)YQR | GTGAAACCAGTAACGTTATACGATGTCGCAGAGTATGCC |
| Note: The DNA-binding | GGTGTCTCTTATCAGACCGTTTCCCGCGTGGTGAACCAG |
| domain YQR is swapped by | GCCAGCCACGTTTCTGCGAAAACGCGGGAAAAAGTGG |
| mutating positions Y17, Q18, | AAGCGGCGATGGCGGAGCTCAATTACATTCCCAACCGC |
| and R22 to confer alternate | GTGGCACAACAACTGGCGGGCAAAGCGTCGCATACCAT |
| DNA-binding | TGGCATGTTGATCACTGCCAGTACCAATCCTTTCTATTCA |
| (e.g., RA(2)KSL | GAACTGGTGCGTGGCGTTGAACGCAGCTGCTTCGAACG |
| would be Y17K, Q18S, and | CGGTTATAGTCTCGTCCCTTGCAATACCGAAGGCGATGA |
| R22L) | ACAGCGGATGAATCGCAATCTGGAAACGCTGATGCAAA |
| | AACGCGTTGATGGCTTGCTGTTACTGTGCACCGAAACG |
| | CATCAACCTTCGCGTGAAATCATGCAACGTTATCCGACA |
| | GTGCCTACTGTGATGATGGACTGGGCTCCGTTCGATGGC |
| | GACAGCGATCTTATTCAGGATAACTCGTTGCTGGGCGGA |
| | GACTTAGCAACGCAATATCTGATCGATAAAGGTCATACC |
| | CGTATCGCCTGTATTACCGGCCCGCTGGATAAAACTCCG |
| | GCGCGCCTGCGGTTGGAAGGTTATCGGGCGGCGATGAA |
| | ACGTGCGGGTCTCAACATTCCTGATGGCTATGAAGTCAC |
| | TGGTGATTTTGAATTTAACGGCGGGTTTGACGCTATGCG |
| | CCAACTGCTATCACATCCGCTGCGTCCTCAGGCCGTCTT |
| | TACCGGAAATGACGCTATGGCTGTTGGCGTTTACCAGGC |
| | GTTATATCAGGCAGAGTTACAGGTTCCGCAGGATATCGC |
| | GGTGATTGGCTATGACGATATCGAACTGGCAAGCTTTAT |
| | GACGCCACCATTAACCACTATCCACCAACCGAAAGATG |
| | AACTGGGGGAGCTGGCGATTGATGTACTCATCCATCGGA |
| | TAACCCAGCCGACCCTTCAGCAACAACGATTACAACTT |
| | ACTCCGATTCTGATGGAACGCGGTTCGGCTTAGATGAAA |
| | CCAGTAACGTTATACGATGTCGCAGAGTATGCCGGTGTC |
| | TCTACCGCGACCGTTTCCAACGTGGTGAACCAGGCCAG |
| | CCACGTTTCTGCGAAAACGCGGGAAAAAGTGGAAGCG |
| | GCGATCAAAGAGCTGGGCTACGTGCCGAACCGCGCAGC |
| | CCGCACCC (SEQ ID NO: 57) |
| E⁺TAN | TGGTCACCCGACGTACCGACACCGTAGCCCTGGTGGTG |
| | TCGGAAAACAACCAGAAGCTCTTCGCCGAACCCTTCTA |
| | TGCCGGGATCGTGCTCGGCGTGGGGGTTGCTCTGTCCG |
| | AACGGGGATTCCAGTTCGTCCTGGCCACGGGCCGCTCC |
| | GGGATAGAGCATGAGCGGCTGGGCGGCTACCTGGCCGG |
| | ACAGCACGTCGACGGGGTCCTCCTGCTGTCGCTCCACC |
| | GCGACGACCCGCTGCCGCAGATGCTGGACGAGGCCGG |
| | GGTGCCGTACGTCTACGGCGGCCGTCCGCTCGGCGTCC |
| | CCGAAGAACAGGTGTCCTATGTCGATATCGACAACATCG |
| | GCGGGGGACGCCAGGCCACCCAGCGGCTGATCGAGAC |
| | CGGGCACCGGCGGATCGCTACGATCGCGGGCCCGCAGG |
| | ACATGGTCGCTGGTGTGGAACGCCTCCAGGGGTATCGC |
| | GAAGCACTGCTCGCCGCGGGGATGGAGTACGACGAAA |
| | CACTGGTGAGCTACGGTGACTTCACCTACGACAGCGGG |
| | GTGGCCGCGATGCGGGAGCTGCTGGATCGGGCCCCCGA |
| | CGTGGACGCCGTGTTCGCGGCCTCCGACTTGATGGGGC |
| | TGGCCGCGCTGCGGGTGCTGCGTGCTTCGGGACGCCGC |
| | GTGCCCGAGGATGTGGCGGTGGTCGGCTACGACGACTC |
| | GACCGTAGCCGAGCACGCCGAACCGCCGATGACCAGCG |
| | TCAACCAGCCCACCGAGCTGATGGGCCGGGAGATGGCC |
| | CGGCTGCTCGTGGACCGGATCACCGGGGAGACCACCGA |
| | ACCGGTGCGGCTGGTGCTGGAGACCCATTTGATGGTGC |
| | GGGAATCCGGGTGA (SEQ ID NO: 58) |
| PhIFAM | ATGGCACGTACCCCGAGCCGTAGCAGCATTGGTAGCCT |
| | GCGTAGTCCGCATACCCATAAAGCAATTCTGACCAGCAC |
| | CATTGAAATCCTGAAAGAATGTGGTTATAGCGGTCTGAG |
| | CATTGAAAGCGTGGCACGTCGCGCCGGTGCAGGCAAAC |
| | CGACCATTTATCGTTGGTGGACCAACAAAGCAGCACTG |
| | ATTGCCGAAGTGTATGAAAATGAAATCGAACAGGTACG |
| | TAAATTTCCGGATTTGGGTAGCTTTAAAGCCGATCTGGA |
| | TTTTCTGCTGCATAATCTGTGGAAAGTTTGGCGTGAAAC |
| | CATTTGTGGTGAAGCATTTCGTTGTGTTATTGCAGAAGC |
| | ACAGTTGGACCCTGTAACCCTGACCCAACTGAAAGATC |
| | AGTTTATGGAACGTCGTCGTGAGATACCGAAAAAACTG |
| | GTTGAAGATGCCATTAGCAATGGTGAACTGCCGAAAGA |
| | TATCAATCGTGAACTGCTGCTGGATATGATTTTTGGTTTT |
| | TGTTGGTATCGCCTGCTGACCGAACAGTTGACCGTTGA |
| | ACAGGATATTGAAGAATTTACCTTCCTGCTGATTAATGGT |
| | GTTTGTCCGGGTACACAGTGTTGA (SEQ ID NO: 59) |
| A118 Recombinase | GTGAAGGCAGCTATTTATATACGTGTTTCTACTCAAGAG |
| | CAAGTAGAAAATTATTCAATACAAGCTCAAACTGAAAA |
| | ACTAACAGGATTGTGCCGCTCGAAGGACTGGGACGTAT |
| | ACGATATTTTCATTGACGGCGGATACTCCGGCTCAAATAT |
| | GAATCGTCCCGCATTAAATGAAATGCTAAGTAAACTACA |
| | CGAAATTGATGCTGTAGTCGTATATCGATTAGACAGACT |
| | ATCCCGCTCACAAAGAGACACAATAACGCTTATTGAAG |
| | AATACTTCTTAAAAAACAATGTAGAGTTTGTTAGTTTGT |
| | CTGAAACGCTTGATACTAGTTCCCCTTTCGGTCGTGCAA |
| | TGATTGGTATATTATCAGTATTCGCACAGCTAGAGCGCG |
| | AAACAATCCGAGATCGTATGGTGATGGGGAAAATTAAG |
| | CGTATTGAAGCAGGTCTTCCGTTAACAACTGCGAAAGG |
| | TAGAACGTTCGGCTATGATGTTATAGATACAAAATTATAC |
| | ATTAATGAAGAAGAAGCAAAACAGTTACAACTGATTTAT |
| | GATATTTTCGAAGAAGAACAAAGTATAACTTTTTTACAG |
| | AAAAGACTAAAAAAATTAGGCTTTAAAGTTAGAACATAT |
| | AATCGCTATAACAACTGGCTAACTAATGATTTGTATTGTG |
| | GTTATGTTTCATATAAAGATAAAGTTCATGTAAAAGGTAT |
| | TCATGAACCTATCATCAGTGAAGAGCAATTCTATAGAGT |
| | TCAAGAAATATTTACTCGTATGGGTAAAAATCCGAACAT |
| | GAATAGAGATTCAGCATCGTTGCTAAATAATTTAGTAGTT |
| | TGTAGTAAATGCGGGTTAGGCTTTGTTCATCGTAGAAAA |
| | GATACAATGTCGCGTGGTAAAAAATATCATTATAGATATT |
| | ATAGTTGCAAGACTTATAAACATACTCATGAACTCGAAA |
| | AATGCGGGAATAAAATTTGGAGAGCTGACAAACTTGAA |
| | GAATTAATTATTAATCGTGTGAATAATTATAGTTTCGCTTC |
| | CAGAAATGTAGATAAAGAAGATGAATTAGATAGCTTAAA |
| | TGAAAAGCTTAAAATAGAACATGCAAAGAAAAAACGAT |
| | TATTTGATTTATATATAAATGGCTCGTATGAAGTTTCAGA |
| | ACTTGATTCTATGATGAATGATATTGATGCTCAAATTAAT |
| | TATTATGAATCACAAATAGAAGCTAACGAAGAATTGAAG |
| | AAAAACAAAAAGATACAAGAAAATTTAGCTGATTTAGC |
| | AACAGTTGATTTTGACTCTTTAGAGTTCAGAGAAAAGC |
| | AACTTTATTTAAAATCACTAATAAACAAAATTTATATTGA |
| | TGGTGAACAAGTTACTATTGAATGGCTC (SEQ ID NO: 60) |
| dCas9 | ATGGATAAGAAATACTCAATAGGCTTAGCTATCGGCACA |
| | AATAGCGTCGGATGGGCGGTGATCACTGATGAATATAAG |
| | GTTCCGTCTAAAAAGTTCAAGGTTCTGGGAAATACAGA |
| | CCGCCACAGTATCAAAAAAAATCTTATAGGGGCTCTTTT |
| | ATTTGACAGTGGAGAGACAGCGGAAGCGACTCGTCTCA |
| | AACGGACAGCTCGTAGAAGGTATACACGTCGGAAGAAT |
| | CGTATTTGTTATCTACAGGAGATTTTTTCAAATGAGATGG |
| | CGAAAGTAGATGATAGTTTCTTTCATCGACTTGAAGAGT |
| | CTTTTTTGGTGGAAGAAGACAAGAAGCATGAACGTCAT |
| | CCTATTTTTGGAAATATAGTAGATGAAGTTGCTTATCATG |
| | AGAAATATCCAACTATCTATCATCTGCGAAAAAAATTGG |
| | TAGATTCTACTGATAAAGCGGATTTGCGCTTAATCTATTT |
| | GGCCTTAGCGCATATGATTAAGTTTCGTGGTCATTTTTTG |
| | ATTGAGGGAGATTTAAATCCTGATAATAGTGATGTGGAC |
| | AAACTATTTATCCAGTTGGTACAAACCTACAATCAATTAT |
| | TTGAAGAAAACCCTATTAACGCAAGTGGAGTAGATGCT |
| | AAAGCGATTCTTTCTGCACGATTGAGTAAATCAAGACG |
| | ATTAGAAAATCTCATTGCTCAGCTCCCCGGTGAGAAGA |
| | AAAATGGCTTATTTGGGAATCTCATTGCTTTGTCATTGGG |
| | TTTGACCCCTAATTTTAAATCAAATTTTGATTTGGCAGAA |
| | GATGCTAAATTACAGCTTTCAAAAGATACTTACGATGAT |
| | GATTTAGATAATTTATTGGCGCAAATTGGAGATCAATATG |
| | CTGATTTGTTTTTGGCAGCTAAGAATTTATCAGATGCTAT |
| | TTTACTTTCAGATATCCTAAGAGTAAATACTGAAATAACT |
| | AAGGCTCCCCTATCAGCTTCAATGATTAAACGCTACGAT |
| | GAACATCATCAAGACTTGACTCTTTTAAAAGCTTTAGTT |
| | CGACAACAACTTCCAGAAAAGTATAAAGAAATCTTTTTT |
| | GATCAATCAAAAAACGGATATGCAGGTTATATTGATGGG |
| | GGAGCTAGCCAAGAAGAATTTTATAAATTTATCAAACCA |
| | ATTTTAGAAAAAATGGATGGTACTGAGGAATTATTGGTG |
| | AAACTAAATCGTGAAGATTTGCTGCGCAAGCAACGGAC |
| | CTTTGACAACGGCTCTATTCCCCATCAAATTCACTTGGG |
| | TGAGCTGCATGCTATTTTGAGAAGACAAGAAGACTTTTA |
| | TCCATTTTTAAAAGACAATCGTGAGAAGATTGAAAAAAT |
| | CTTGACTTTTCGAATTCCTTATTATGTTGGTCCATTGGCG |
| | CGTGGCAATAGTCGTTTTGCATGGATGACTCGGAAGTCT |
| | GAAGAAACAATTACCCCATGGAATTTTGAAGAAGTTGT |
| | CGATAAAGGTGCTTCAGCTCAATCATTTATTGAACGCAT |
| | GACAAACTTTGATAAAAATCTTCCAAATGAAAAAGTAC |
| | TACCAAAACATAGTTTGCTTTATGAGTATTTTACGGTTTA |
| | TAACGAATTGACAAAGGTCAAATATGTTACTGAAGGAAT |
| | GCGAAAACCAGCATTTCTTTCAGGTGAACAGAAGAAAG |
| | CCATTGTTGATTTACTCTTCAAAACAAATCGAAAAGTAA |
| | CCGTTAAGCAATTAAAAGAAGATTATTTCAAAAAAATAG |
| | AATGTTTTGATAGTGTTGAAATTTCAGGAGTTGAAGATA |
| | GATTTAATGCTTCATTAGGTACCTACCATGATTTGCTAAA |
| | AATTATTAAAGATAAAGATTTTTTGGATAATGAAGAAAA |
| | TGAAGATATCTTAGAGGATATTGTTTTAACATTGACCTTA |
| | TTTGAAGATAGGGAGATGATTGAGGAAAGACTTAAAAC |
| | ATATGCTCACCTCTTTGATGATAAGGTGATGAAACAGCT |
| | TAAACGTCGCCGTTATACTGGTTGGGGACGTTTGTCTCG |
| | AAAATTGATTAATGGTATTAGGGATAAGCAATCTGGCAA |
| | AACAATATTAGATTTTTTGAAATCAGATGGTTTTGCCAAT |
| | CGCAATTTTATGCAGCTGATCCATGATGATAGTTTGACAT |
| | TTAAAGAAGACATTCAAAAAGCACAAGTGTCTGGACAA |
| | GGCGATAGTTTACATGAACATATTGCAAATTTAGCTGGT |
| | AGCCCTGCTATTAAAAAAGGTATTTTACAGACTGTAAAA |
| | GTTGTTGATGAATTGGTCAAAGTAATGGGGGGCATAAG |
| | CCAGAAAATATCGTTATTGAAATGGCACGTGAAAATCAG |
| | ACAACTCAAAAGGGCCAGAAAAATTCGCGAGAGCGTAT |
| | GAAACGAATCGAAGAAGGTATCAAAGAATTAGGAAGTC |
| | AGATTCTTAAAGAGCATCCTGTTGAAAATACTCAATTGC |
| | AAAATGAAAAGCTCTATCTCTATTATCTCCAAAATGGAA |
| | GAGACATGTATGTGGACCAAGAATTAGATATTAATCGTT |
| | TAAGTGATTATGATGTCGATGCCATTGTTCCACAAAGTTT |
| | CCTTAAAGACGATTCAATAGACAATAAGGTCTTAACGCG |
| | TTCTGATAAAAATCGTGGTAAATCGGATAACGTTCCAAG |
| | TGAAGAAGTAGTCAAAAAGATGAAAAACTATTGGAGAC |
| | AACTTCTAAACGCCAAGTTAATCACTCAACGTAAGTTTG |
| | ATAATTTAACGAAAGCTGAACGTGGTGGTTTGAGTGAA |
| | CTTGATAAAGCTGGTTTTATCAAACGCCAATTGGTTGAA |
| | ACTCGCCAAATCACTAAGCATGTGGCACAAATTTTGGAT |
| | AGTCGCATGAATACTAAATACGATGAAAATGATAAACTT |
| | ATTCGAGAGGTTAAAGTGATTACCTTAAAATCTAAATTA |
| | GTTTCTGACTTCCGAAAAGATTTCCAATTCTATAAAGTA |
| | CGTGAGATTAACAATTACCATCATGCCCATGATGCGTATC |
| | TAAATGCCGTCGTTGGAACTGCTTTGATTAAGAAATATC |
| | CAAAACTTGAATCGGAGTTTGTCTATGGTGATTATAAAG |
| | TTTATGATGTTCGTAAAATGATTGCTAAGTCTGAGCAAG |
| | AAATAGGCAAAGCAACCGCAAAATATTTCTTTTACTCTA |
| | ATATCATGAACTTCTTCAAAACAGAAATTACACTTGCAA |
| | ATGGAGAGATTCGCAAACGCCCTCTAATCGAAACTAATG |
| | GGGAAACTGGAGAAATTGTCTGGGATAAAGGGCGAGAT |
| | TTTGCCACAGTGCGCAAAGTATTGTCCATGCCCCAAGTC |
| | AATATTGTCAAGAAAACAGAAGTACAGACAGGCGAATT |
| | CTCCAAGGAGTCAATTTTACCAAAAAGAAATTCGGACA |
| | AGCTTATTGCTCGTAAAAAAGACTGGGATCCAAAAAAA |
| | TATGGTGGTTTTGATAGTCCAACGGTAGCTTATTCAGTCC |
| | TAGTGGTTGCTAAGGTGGAAAAAGGGAAATCGAAGAA |
| | GTTAAAATCCGTTAAAGAGTTACTAGGGATCACAATTAT |
| | GGAAAGAAGTTCCTTTGAAAAAAATCCGATTGACTTTT |
| | TAGAAGCTAAAGGATATAAGGAAGTTAAAAAAGACTTA |
| | ATCATTAAACTACCTAAATATAGTCTTTTTGAGTTAGAAA |
| | ACGGTCGTAAACGGATGCTGGCTAGTGCCGGAGAATTA |
| | CAAAAAGGAAATGAGCTGGCTCTGCCAAGCAAATATGT |
| | GAATTTTTTATATTTAGCTAGTCATTATGAAAAGTTGAAG |
| | GGTAGTCCAGAAGATAACGAACAAAAACAATTGTTTGT |
| | GGAGCAGCATAAGCATTATTTAGATGAGATTATTGAGCA |
| | AATCAGTGAATTTTCTAAGCGTGTTATTTTAGCAGATGCC |
| | AATTTAGATAAAGTTCTTAGTGCATATAACAAACATAGA |
| | GACAAACCAATACGTGAACAAGCAGAAAATATTATTCAT |
| | TTATTTACGTTGACGAATCTTGGAGCTCCCGCTGCTTTTA |
| | AATATTTTGATACAACAATTGATCGTAAACGATATACGTC |
| | TACAAAAGAAGTTTTAGATGCCACTCTTATCCATCAATC |
| | CATCACTGGTCTTTATGAAACACGCATTGATTTGAGTCA |
| | GCTAGGAGGTGACTGA (SEQ ID NO: 61) |
| engRBS3 sgRNA (non-coding) | CTCATGAATACCTCCTTAAGGTTTTAGAGCTAGAAATAG |
| | CAAGTTAAAATAAGGCTAGTCCGTTATCAACTTGAAAAA |
| | GTGGCACCGAGTCGGTGCTTTTTT (SEQ ID NO: 62) |
| BBa_B0034 | TGGTACCTTTCTCCTCTTTAGTTTTAGAGCTAGAAATAGC |
| sgRNA (non-coding) | AAGTTAAAATAAGGCTAGTCCGTTATCAACTTGAAAAA |
| | GTGGCACCGAGTCGGTGCTTTTTT (SEQ ID NO: 63 |

### REFERENCES

[1]. Guet, C. C., Elowitz, M. B., Hsing, W. H. and Leibler, S. (2002) Combinatorial synthesis of genetic networks. Science, 296, 1466-1470.
[2]. Meyer, A. J., Segall-Shapiro, T. H., Glassey, E., Zhang, J., and Voigt, C. A. (2019). Escherichia coli “Marionette” strains with 12 highly optimized small-molecule sensors. Nat Chem Biol, 15, 196-+.
[3]. Chen, Y, Zhang, S. Y, Young, E. M., Jones, T. S., Densmore, D. and Voigt, C. A. (2020) Genetic circuit design automation for yeast. Nat Microbiol, 5, 1349-+.
[4]. Taketani, M., Zhang, J., Zhang, S., Triassi, A. J., Huang, Y J., Griffith, L. G. and Voigt, C. A. (2020) Genetic circuit design automation for the gut resident species Bacteroides thetaiotaomicron. Nat Biotechnol, 38, 962-969.
[5]. Nielsen, A. A., Der, B. S., Shin, J., Vaidyanathan, P., Paralanov, V., Strychalski, E. A., Ross, D., Densmore, D. and Voigt, C. A. (2016) Genetic circuit design automation. Science, 352, aac7341.
[6]. Auslander, D., Auslander, S., Pierrat, X., Hellmann, L., Rachid, L. and Fussenegger, M. (2018) Programmable full-adder computations in communicating three-dimensional cell cultures. Nat Methods, 15, 57-60.
[7]. Zhou, Z., Liu, Y, Feng, Y, Klepin, S., Tsimring, L. S., Pillus, L., Hasty, J. and Hao, N. (2023) Engineering longevity-design of a synthetic gene oscillator to slow cellular aging. Science, 380, 376-381.
[8]. Wu, Y, Li, Y, Jin, K., Zhang, L., Li, J., Liu, Y, Du, G., Lv, X., Chen, J., Ledesma-Amaro, R. et al. (2023) CRISPR-dCas12a-mediated genetic circuit cascades for multiplexed pathway optimization. Nat Chem Biol, 19, 367-377.
[9]. Rondon, R. E., Groseclose, T. M., Short, A. E. and Wilson, C. J. (2019) Transcriptional programming using engineered systems of transcription factors and genetic architectures. Nat Commun, 10.
[10]. Groseclose, T. M., Rondon, R. E., Herde, Z. D., Aldrete, C. A. and Wilson, C. J. (2020) Engineered systems of inducible anti-repressors for the next generation of biological programming. Nat Commun, 11.
[11]. Milner, P. T., Zhang, Z. Q., Herde, Z. D., Vedire, N. R., Zhang, F. M., Realff, M. J. and Wilson, C. J. (2023) Performance Prediction of Fundamental Transcriptional Programs. Acs Synth Biol, 12, 1094-1108.
[12]. Shin, J., Zhang, S., Der, B. S., Nielsen, A. A. and Voigt, C. A. (2020) Programming Escherichia coli to function as a digital display. Mol Syst Biol, 16, e9401.
[13]. Huang, B. D., Groseclose, T. M. and Wilson, C. J. (2022) Transcriptional programming in a Bacteroides consortium. Nat Commun, 13, 3901.
[14]. Zhu, R., Del Rio-Salgado, J. M., Garcia-Ojalvo, J. and Elowitz, M. B. (2022) Synthetic multistability in mammalian cells. Science, 375, eabg9765.
[15]. Park, J. H., Bassalo, M. C., Lin, G. M., Chen, Y, Doosthosseini, H., Schmitz, J., Roubos, J. A. and Voigt, C. A. (2023) Design of Four Small-Molecule-Inducible Systems in the Yeast Chromosome, Applied to Optimize Terpene Biosynthesis. Acs Synth Biol, 12, 1119-1132.
[16]. Zhang, F., Carothers, J. M. and Keasling, J. D. (2012) Design of a dynamic sensor-regulator system for production of chemicals and fuels derived from fatty acids. Nat Biotechnol, 30, 354-359.
[17]. Mannan, A. A. and Bates, D. G. (2021) Designing an irreversible metabolic switch for scalable induction of microbial chemical production. Nat Commun, 12, 3419.
[18]. Xu, X., Li, X., Liu, Y, Zhu, Y, Li, J., Du, G., Chen, J., Ledesma-Amaro, R. and Liu, L. (2020) Pyruvate-responsive genetic circuits for dynamic control of central metabolism. Nat Chem Biol, 16, 1261-1268.
[19]. Moser, F., Espah Borujeni, A., Ghodasara, A. N., Cameron, E., Park, Y and Voigt, C. A. (2018) Dynamic control of endogenous metabolism with combinatorial logic circuits. Mol Syst Biol, 14, e8605.
[20]. Kang, C. W., Lim, H. G., Won, J., Cha, S., Shin, G., Yang, J. S., Sung, J. and Jung, G. Y (2022) Circuit-guided population acclimation of a synthetic microbial consortium for improved biochemical production. Nat Commun, 13, 6506.
[21]. Chan, C. T. Y, Lee, J. W., Cameron, D. E., Bashor, C. J. and Collins, J. J. (2016)‘Deadman’ and ‘Passcode’ microbial kill switches for bacterial containment. Nat Chem Biol, 12, 82-+.
[22]. Stirling, F., Bitzan, L., O'Keefe, S., Redfield, E., Oliver, J. W. K., Way, J. and Silver, P. A. (2017) Rational Design of Evolutionarily Stable Microbial Kill Switches. Mol Cell, 68, 686-697 e683.
[23]. Xue, Y B., Du, P., Shendi, A. A. I. and Yu, B. (2022) Mercury bioremediation in the aquatic environment by genetically modified bacteria with self-controlled biosecurity circuit. J Clean Prod, 337.
[24]. Brophy, J. A. N., Magallon, K. J., Duan, L., Zhong, V., Ramachandran, P., Kniazev, K. and Dinneny, J. R. (2022) Synthetic genetic circuits as a means of reprogramming plant roots. Science, 377, 747-751.
[25]. Ryu, M. H., Zhang, J., Toth, T., Khokhani, D., Geddes, B. A., Mus, F., Garcia-Costas, A., Peters, J. W., Poole, P. S., Ane, J. M. et al. (2020) Control of nitrogen fixation in bacteria that associate with cereals. Nat Microbiol, 5, 314-330.
[26]. Lloyd, J. P. B., Ly, F., Gong, P., Pflueger, J., Swain, T., Pflueger, C., Fourie, E., Khan, M. A., Kidd, B. N. and Lister, R. (2022) Synthetic memory circuits for stable cell reprogramming in plants. Nat Biotechnol, 40, 1862-1872.
[27]. Triassi, A. J., Fields, B. D., Monahan, C. E., Means, J. M., Park, Y, Doosthosseini, H., Padmakumar, J. P., Isabella, V. M. and Voigt, C. A. (2023) Redesign of an Escherichia coli Nissle treatment for phenylketonuria using insulated genomic landing pads and genetic circuits to reduce burden. Cell Syst, 14, 512-524 e512.
[28]. Abedi, M. H., Yao, M. S., Mittelstein, D. R., Bar-Zion, A., Swift, M. B., Lee-Gosselin, A., Barturen-Larrea, P., Buss, M. T. and Shapiro, M. G. (2022) Ultrasound-controllable engineered bacteria for cancer immunotherapy. Nat Commun, 13, 1585.
[29]. Deng, F., Pan, J., Liu, Z., Zeng, L. and Chen, J. (2023) Programmable DNA biocomputing circuits for rapid and intelligent screening of SARS-CoV-2 variants. Biosens Bioelectron, 223, 115025.
[30]. Li, H. S., Israni, D. V, Gagnon, K. A., Gan, K. A., Raymond, M. H., Sander, J. D., Roybal, K. T., Joung, J. K., Wong, W. W. and Khalil, A. S. (2022) Multidimensional control of therapeutic human cell function with synthetic gene circuits. Science, 378, 1227-1234.
[31]. Xie, M. and Fussenegger, M. (2018) Designing cell function: assembly of synthetic gene circuits for cell biology applications. Nat Rev Mol Cell Biol, 19, 507-525.
[32]. Grozinger, L., Amos, M., Gorochowski, T. E., Carbonell, P., Oyarzun, D. A., Stoof, R., Fellermann, H., Zuliani, P., Tas, H. and Goni-Moreno, A. (2019) Pathways to cellular supremacy in biocomputing. Nat Commun, 10.
[33]. Thapliyal, H. and Ranganathan, N. (2010) Design of reversible sequential circuits optimizing quantum cost, delay, and garbage outputs. ACM Journal on Emerging Technologies in Computing Systems, 6, 31.
[34]. Peres, A. (1985) Reversible logic and quantum computers. Phys Rev A Gen Phys, 32, 3266-3276.
[35]. Takeuchi, N., Yamanashi, Y and Yoshikawa, N. (2014) Reversible logic gate using adiabatic superconducting devices. Sci Rep, 4, 6354.
[36]. Rao, D. G. S., Swarnakar, S. and Kumar, S. (2020) Design of all-optical reversible logic gates using photonic crystal waveguides for optical computing and photonic integrated circuits. Appl Opt, 59, 11003-11012.
[37]. Choudhary, K. and Kumar, S. (2021) Optimized plasmonic reversible logic gate for low loss communication. Appl Opt, 60, 4567-4572.
[38]. Orbach, R., Remacle, F., Levine, R. D. and Willner, I. (2012) Logic reversibility and thermodynamic irreversibility demonstrated by DNAzyme-based Toffoli and Fredkin logic gates. Proc Natl Acad Sci USA, 109, 21228-21233.
[39]. Fratto, B. E. and Katz, E. (2016) Controlled Logic Gates-Switch Gate and Fredkin Gate Based on Enzyme-Biocatalyzed Reactions Realized in Flow Cells. Chemphyschem, 17, 1046-1053.
[40]. Sarkar, K., Bonnerjee, D., Srivastava, R. and Bagh, S. (2021) A single layer artificial neural network type architecture with molecular engineered bacteria for reversible and irreversible computing. Chem Sci, 12, 15821-15832.
[41]. Srivastava, R. and Bagh, S. (2023) A Logically Reversible Double Feynman Gate with Molecular Engineered Bacteria Arranged in an Artificial Neural Network-Type Architecture. Acs Synth Biol, 12, 51-60.
[42]. Srivastava, R., Sarkar, K., Bonnerjee, D. and Bagh, S. (2022) Synthetic Genetic Reversible Feynman Gate in a Single E. coli Cell and Its Application in Bacterial to Mammalian Cell Information Transfer. Acs Synth Biol, 11, 1040-1048.
[43]. Short, A. E., Kim, D., Milner, P. T. and Wilson, C. J. (2023) Next generation synthetic memory via intercepting recombinase function. Nat Commun, 14, 5255.
[44]. Huang, B. D., Kim, D., Yu, Y and Wilson, C. J. (2024) Engineering intelligent chassis cells via recombinase-based MEMORY circuits. Nat Commun, 15, 2418.
[45]. Rondon, R. E. and Wilson, C. J. (2019) Engineering a New Class of Anti-Lac Transcription Factors with Alternate DNA Recognition. Acs Synth Biol, 8, 307-317.
[46]. Warman, E. A., Forrest, D., Guest, T., Haycocks, J., Wade, J. T. and Grainger, D. C. (2021) Widespread divergent transcription from bacterial and archaeal promoters is a consequence of DNA-sequence symmetry. Nat Microbiol, 6, 746-756.
[47]. Davey, J. A. and Wilson, C. J. (2020) Engineered signal-coupled inducible promoters: measuring the apparent RNA-polymerase resource budget. Nucleic Acids Res, 48, 9995-10012.
[48]. Dong, F., Spott, S., Zimmermann, O., Kisters-Woike, B., Muller-Hill, B. and Barker, A. (1999) Dimerisation mutants of Lac repressor. I. A monomeric mutant, L251A, that binds Lac operator DNA as a dimer. J Mol Biol, 290, 653-666.
[49]. Chen, J. and Matthews, K. S. (1992) Deletion of lactose repressor carboxyl-terminal domain affects tetramer formation. J Biol Chem, 267, 13843-13850.
[50]. Marinescu, D. C. and Marinescu, G. M. (2012) Classical and quantum information. Academic Press, Burlington, M A.
[51]. Fedorov, A., Steffen, L., Baur, M., da Silva, M. P. and Wallraff, A. (2011) Implementation of a Toffoli gate with superconducting circuits. Nature, 481, 170-172.
[52]. Siuti, P., Yazbek, J. and Lu, T. K. (2013) Synthetic circuits integrating logic and memory in living cells. Nat Biotechnol, 31, 448-452.
[53]. Yang, L., Nielsen, A. A., Fernandez-Rodriguez, J., McClune, C. J., Laub, M. T., Lu, T. K. and Voigt, C. A. (2014) Permanent genetic memory with >1-byte capacity. Nat Methods, 11, 1261-1266.
[54]. Guiziou, S., Mayonove, P. and Bonnet, J. (2019) Hierarchical composition of reliable recombinase logic devices. Nat Commun, 10, 456.
[55]. Stanton, B. C., Nielsen, A. A. K., Tamsir, A., Clancy, K., Peterson, T. and Voigt, C. A. (2014) Genomic mining of prokaryotic repressors for orthogonal logic gates. Nat Chem Biol, 10, 99-105.
[56]. Loessner, M. J., Inman, R. B., Lauer, P. and Calendar, R. (2000) Complete nucleotide sequence, molecular analysis and genome structure of bacteriophage A118 of Listeria monocytogenes: implications for phage evolution. Mol Microbiol, 35, 324-340.
[57]. Hersey, A. N., Kay, V. E., Lee, S., Realff, M. J. and Wilson, C. J. (2023) Engineering allosteric transcription factors guided by the LacI topology. Cell Syst, 14, 645-655.
[58]. Tomasello, G., Armenia, I. and Molla, G. (2020) The Protein Imager: a full-featured online molecular viewer interface with server-side H Q-rendering capabilities. Bioinformatics, 36, 2909-2911.
[59]. Zhan, H., Camargo, M. and Matthews, K. S. (2010) Positions 94-98 of the lactose repressor Nsubdomain monomer-monomer interface are critical for allosteric communication. Biochemistry, 49, 8636-8645.
[60]. Calos, M. P. (1978) DNA sequence for a low-level promoter of the lac repressor gene and an ‘up’ promoter mutation. Nature, 274, 762-765.
[61]. de Boer, H. A., Comstock, L. J. and Vasser, M. (1983) The tac promoter: a functional hybrid derived from the trp and lac promoters. Proc Natl Acad Sci USA, 80, 21-25.
[62]. Elowitz, M. B. and Leibler, S. (2000) A synthetic oscillatory network of transcriptional regulators. Nature, 403, 335-338.
[63]. Salis, H. M., Mirsky, E. A. and Voigt, C. A. (2009) Automated design of synthetic ribosome binding sites to control protein expression. Nat Biotechnol, 27, 946-950.
[64]. Lou, C., Stanton, B., Chen, Y J., Munsky, B. and Voigt, C. A. (2012) Ribozyme-based insulator parts buffer synthetic circuits from genetic context. Nat Biotechnol, 30, 1137-1142.
[65]. Dufour, D., de la Pena, M., Gago, S., Flores, R. and Gallego, J. (2009) Structure-function analysis of the ribozymes of Chrysanthemum chlorotic mottle viroid: a loop-loop interaction motif conserved in most natural hammerheads. Nucleic Acids Res, 37, 368-381.
[66]. Khvorova, A., Lescoute, A., Westhof, E. and Jayasena, S. D. (2003) Sequence elements outside the hammerhead ribozyme catalytic core enable intracellular activity. Nat Struct Biol, 10, 708-712.
[67]. Pley, H. W., Flaherty, K. M. and McKay, D. B. (1994) Three-dimensional structure of a hammerhead ribozyme. Nature, 372, 68-74.
[68]. Gao, Y and Zhao, Y (2014) Self-processing of ribozyme-flanked RNAs into guide RNAs in vitro and in vivo for CRISPR-mediated genome editing. J Integr Plant Biol, 56, 343-349.
[69]. Ferre-D'Amare, A. R., Zhou, K. and Doudna, J. A. (1998) Crystal structure of a hepatitis delta virus ribozyme. Nature, 395, 567-574.
[70]. Chen, Y J., Liu, P., Nielsen, A. A., Brophy, J. A., Clancy, K., Peterson, T. and Voigt, C. A. (2013) Characterization of 582 natural and synthetic terminators and quantification of their design constraints. Nat Methods, 10, 659-664.

SEQUENCES
PlacI
SEQ ID NO: 1
GCGCAAAACCTTTCGCGGTATGGCATGAT
PlacIQ
SEQ ID NO: 2
GTGCAAAACCTTTCGCGGTATGGCATGAT
PRA2HQN
SEQ ID NO: 3
CGCCAAAACCTTTCGCGGTATGGCATGAT
Pta
SEQ ID NO: 4
TTGACATTTTAAGCGCTTAAAATTATAAT
PmJ23119
SEQ ID NO: 5
TGTACAGCTAGCTCAGTCCTAGGTATAAT
PTrc
SEQ ID NO: 6
TTGACAATTAATCATCCGGCTCGTATAATG
PPh1F
SEQ ID NO: 7
TTGACATGATACGAAACGTACCGTATCGTTAAGGT
PdCas9
SEQ ID NO: 8
TTGACAGATAGTTGCATCTGTGCTATAAT
PBD(35)rep
SEQ ID NO: 9
ATTATACCTAGGACTGAGCTAGCTTGCAAATTAATCATCCGGCTCGTATAAT
PBD(35)lac0
SEQ ID NO: 10
ATCATGCCATACCGCGAAAGGTTTGTACAAACCTTTCGCGGTATGGCATGAT
PBD(35)lac1
SEQ ID NO: 11
ATCATGCCATATACTGAAAGGTTTGTACAAACCTTTCGCGGTATGGCATGAT
PBD(35)lac2
SEQ ID NO: 12
ATCATGCCATAAAATTAAAGGTTTGTACAAACCTTTCGCGGTATGGCATGAT
OSYM
SEQ ID NO: 13
AATTGTGAGCGCTCACAATT
Oagg
SEQ ID NO: 14
AATTAGGAGCGCTCCTAATT
Ottg
SEQ ID NO: 15
AATTTTGAGCGCTCAAAATT
Otta
SEQ ID NO: 16
AATTTTAAGCGCTTAAAATT
Ogta
SEQ ID NO: 17
AATTGTAAGCGCTTACAATT
lac
SEQ ID NO: 18
GGAAGAGAGTCAATTCAGGGTGGTGAAT
BBa_B0034
SEQ ID NO: 19
AAAGAGGAGAAA
C12
SEQ ID NO: 20
GCTGGGAAGGAATACGTAAGAATTA
ph12
SEQ ID NO: 21
GGAAGAGAGTCAATTCATGGGGGTGAAT
H8
SEQ ID NO: 22
GGAAGAGAGTCAATTCATGGGGGTGAAT
E21
SEQ ID NO: 23
GCTGGGAAGGAATCCGTATGAATTAGCTGGGAAGGAATGCGTAAGAATTA
F9
SEQ ID NO: 24
CCTGGGAAGGAATGCGTAGGAATTA
a118
SEQ ID NO: 25
CAACCATCGTATTGAAGGTGGCTAAC
prgJ
SEQ ID NO: 26
CTTTAATTTAACGTAAATAAGGAAGTCATT
eng1
SEQ ID NO: 27
AACAAAATGAGGAGGTACTGAG
eng3
SEQ ID NO: 28
ACAACTTAAGGAGGTATTC
eng5
SEQ ID NO: 29
CTGGTGAGGAGGTGAGA
eng6
SEQ ID NO: 30
AAAGTAAGGAGGCGCGGCT
RiboJ
SEQ ID NO: 31
AGCTGTCACCGGATGTGCTTTCCGGTCTGATGAGTCCGTGAGGACGAAACAGCCTCT
ACAAATAATTTTGTTTAA
RiboJ10
SEQ ID NO: 32
AGCGCTCAACGGGTGTGCTTCCCGTTCTGATGAGTCCGTGAGGACGAAAGCGCCTCT
ACAAATAATTTTGTTTAA
AraJ
SEQ ID NO: 33
AGTGGTCGTGATCTGAAACTCGATCACCTGATGAGCTCAAGGCAGAGCGAAACCACC
TCTACAAATAATTTTGTTTAA
BydvJ
SEQ ID NO: 34
AGGGTGTCTCAAGGTGCGTACCTTGACTGATGAGTCCGAAAGGACGAAACACCCCTC
TACAAATAATTTTGTTTAA
ElvJ
SEQ ID NO: 35
AGCCCCATAGGGTGGTGTGTACCACCCCTGATGAGTCCAAAAGGACGAAATGGGGCC
TCTACAAATAATTTTGTTTAA
PlmJ
SEQ ID NO: 36
AGTCATAAGTCTGGGCTAAGCCCACTGATGAGTCGCTGAAATGCGACGAAACTTATG
ACCTCTACAAATAATTTTGTTTAA
CchJ
SEQ ID NO: 37
AGTTCCAGTCGAGACCTGAAGTGGGTTTCCTGATGAGGCTGTGGAGAGAGCGAAAG
CTTTACTCCCGCACAAGCCGAAACTGGAACCTCTACAAATAATTTTGTTTAA
HH1
SEQ ID NO: 38
CATGAGCTGATGAGTCCGTGAGGACGAAACGAGTAAGCTCGTC
HH2
SEQ ID NO: 39
GCAGTAAGCTGCCTGAAGAGCTGAGTGCAGCGAAAAAAA
HH3
SEQ ID NO: 40
GTACCACTGATGAGTCCGTGAGGACGAAACGAGTAAGCTCGTC
HDV
SEQ ID NO: 41
GGCCGGCATGGTCCCAGCCTCCTCGCTGGCGCCGGCTGGGCAACATGCTTCGGCATG
GCGAATGGGAC
rrnB T1
SEQ ID NO: 42
CAAATAAAACGAAAGGCTCAGTCGAAAGACTGGGCCTTTCGTTTTATCTGTTGTTTGT
CGGTGAACGCTCTC
L3S2P11
SEQ ID NO: 43
CTCGGTACCAAATTCCAGAAAAGAGACGCTTTCGAGCGTCTTTTTTCGTTTTGGTCC
ECK120029600
SEQ ID NO: 44
TTCAGCCAAAAAACTTAAGACCGCCGGTCTTGTCCACTACCTTGCAGTAATGCGGTG
GACAGGATCGGCGGTTTTCTTTTCTCTTCTCAA
ECK120033737
SEQ ID NO: 45
GGAAACACAGAAAAAAGCCCGCACCTGACAGTGCGGGCTTTTTTTTTCGACCAAAG
G
ECK120033736
SEQ ID NO: 46
AACGCATGAGAAAGCCCCCGGAAGATCACCTTCCGGGGGCTTTTTTATTGCGC
ECK120010818
SEQ ID NO: 47
GTCAGTTTCACCTGTTTTACGTAAAAACCCGCTTCGGCGGGTTTTTACTTTTGG
sfGFP (GFP)
SEQ ID NO: 48
ATGAGCAAAGGAGAAGAACTTTTCACTGGAGTTGTCCCAATTCTTGTTGAATTAGATG
GTGATGTTAATGGGCACAAATTTTCTGTCCGTGGAGAGGGTGAAGGTGATGCTACAA
ACGGAAAGCTTACCCTTAAATTTATTTGCACTACTGGAAAACTACCTGTTCCATGGCC
AACACTTGTCACTACTCTCACTTATGGTGTTCAATGCTTTTCCCGTTATCCGGATCATAT
GAAACGGCATGACTTTTTCAAGAGTGCCATGCCCGAAGGTTATGTACAGGAACGCAC
TATATCTTTCAAAGATGACGGGACTTACAAGACGCGTGCTGAAGTCAAGTTTGAAGG
TGATACCCTTGTTAATCGTATCGAGTTAAAAGGTATTGATTTTAAAGAAGATGGAAAC
ATTCTCGGACACAAACTTGAGTACAACTTTAACTCACACAATGTATACATCACGGCAG
ACAAACAAAAGAATGGAATCAAAGCTAACTTCAAAATTCGCCACAACGTTGAAGATG
GATCCGTTCAACTAGCAGACCATTATCAACAAAATACTCCAATTGGCGATGGCCCTGT
CCTTTTACCAGACAACCATTACCTGTCGACACAATCTGTCCTTTCGAAAGATCCCAAC
GAAAAGCGTGACCACATGGTCCTTCTTGAGTTTGTAACTGCTGCTGGGATTACACATG
GCATGGATGAGCTCTACAAA
mCherry (RFP)
SEQ ID NO: 49
ATGGTTAGCAAGGGCGAGGAAGACAACATGGCGATCATCAAAGAGTTCATGCGTTTT
AAGGTTCACATGGAAGGTTCTGTTAACGGTCACGAGTTCGAAATCGAAGGTGAAGGC
GAAGGCCGTCCGTACGAAGGTACCCAGACCGCGAAACTCAAGGTTACCAAAGGTGG
TCCGCTGCCGTTCGCGTGGGACATCCTCTCTCCGCAATTCATGTACGGTTCTAAAGCG
TACGTTAAACATCCGGCAGATATCCCGGACTACCTGAAACTGTCTTTCCCGGAAGGCT
TCAAATGGGAACGTGTTATGAACTTTGAAGACGGCGGTGTTGTTACCGTTACCCAGG
ACTCTTCTCTGCAGGACGGTGAATTTATCTATAAGGTTAAACTCCGTGGTACTAACTTC
CCGTCTGACGGTCCGGTTATGCAGAAAAAGACCATGGGTTGGGAAGCGTCTTCTGAA
CGTATGTACCCGGAGGATGGTGCGCTGAAAGGCGAGATCAAACAGCGTCTCAAACTG
AAAGACGGTGGTCACTACGACGCGGAAGTAAAAACCACCTACAAAGCTAAAAAGCC
AGTTCAGCTGCCGGGTGCGTACAACGTGAACATTAAACTGGACATCACCTCTCACAA
CGAAGACTACACCATTGTTGAACAGTACGAACGTGCGGAGGGTCGTCACTCTACCGG
TGGTATGGACGAACTGTACAAATAA
tagBFP2 (BFP)
SEQ ID NO: 50
ATGAGCGAGCTGATTAAGGAGAACATGCACATGAAGCTGTACATGGAGGGCACCGTG
GACAACCATCACTTCAAGTGCACATCCGAGGGCGAAGGCAAGCCCTACGAGGGCAC
CCAGACCATGAGAATCAAGGTGGTCGAGGGCGGCCCTCTCCCCTTCGCCTTCGACAT
CCTGGCTACTAGCTTCCTCTACGGCAGCAAGACCTTCATCAACCACACCCAGGGCATC
CCCGACTTCTTCAAGCAGTCCTTCCCTGAGGGCTTCACATGGGAGAGAGTCACCACA
TACGAAGACGGGGGCGTGCTGACCGCTACCCAGGACACCAGCCTCCAGGACGGCTG
CCTCATCTACAACGTCAAGATCAGAGGGGTGAACTTCACATCCAACGGCCCTGTGAT
GCAGAAGAAAACACTCGGCTGGGAGGCCTTCACCGAGACGCTGTACCCCGCTGACG
GCGGCCTGGAAGGCAGAAACGACATGGCCCTGAAGCTCGTGGGCGGGAGCCATCTG
ATCGCAAACATCAAGACCACATATAGATCCAAGAAACCCGCTAAGAACCTCAAGATG
CCTGGCGTCTACTATGTGGACTACAGACTGGAAAGAATCAAGGAGGCCAACAACGAG
ACCTACGTCGAGCAGCACGAGGTGGCAGTGGCCAGATACTGCGACCTCCCTAGCAAA
CTGGGGCACTAA
phi YFP (YFP)
SEQ ID NO: 51
ATGAGCTCCGGCGCGCTGCTGTTTCACGGTAAGATCCCGTACGTGGTTGAGATGGAG
GGCAACGTTGACGGTCATACCTTCTCCATCCGTGGTAAAGGTTATGGCGACGCTAGCG
TAGGTAAAGTGGACGCGCAGTTCATCTGCACTACTGGTGACGTGCCGGTTCCGTGGT
CTACTCTGGTGACTACCCTGACCTATGGTGCCCAGTGCTTCGCCAAATACGGTCCAGA
ACTGAAAGATTTCTACAAAAGCTGCATGCCTGAAGGCTATGTTCAGGAACGTACCATT
ACCTTCGAAGGCGACGGTGTGTTCAAAACCCGCGCGGAAGTAACCTTTGAAAACGG
CTCCGTTTACAACCGCGTGAAACTGAACGGTCAAGGCTTCAAAAAAGACGGTCACG
TGCTGGGCAAGAACCTGGAATTCAACTTTACCCCGCACTGCCTGTACATCTGGGGTG
ACCAGGCGAACCATGGCCTGAAATCTGCGTTCAAGATCATGCACGAGATCACTGGCT
CTAAGGAAGACTTTATCGTGGCCGACCACACTCAGATGAACACGCCAATTGGCGGTG
GTCCGGTTCACGTGCCGGAATATCACCACATCACGTACCACGTTACCCTGTCCAAAGA
CGTGACTGATCACCGTGACAACATGTCTCTGGTTGAAACTGTTCGTGCGGTGGATTGC
CGTAAAACGTACCTGTAA
I⁺YQR
SEQ ID NO: 52
ATGAAACCGGTAACGCTTTACGACGTAGCCGAATACGCCGGGGTATCCTACCAGACC
GTGAGTCGTGTCGTAAATCAGGCCTCCCATGTAAGCGCTAAGACAAGAGAGAAAGTG
GAAGCGGCTATGGCAGAGTTGAATTACATCCCCAACCGTGTCGCCCAACAGCTAGCG
GGTAAACAGTCATTACTTATCGGCGTTGCGACAAGCTCTTTGGCGTTACACGCCCCCA
GTCAGATAGTTGCAGCCATCAAGAGTAGGGCTGACCAGCTAGGCGCAAGTGTCGTAG
TGTCAATGGTTGAGAGATCAGGGGTCGAGGCTTGTAAGACGGCTGTGCATAACTTAC
TTGCCCAAAGAGTCTCTGGTTTAATAATAAATTACCCATTGGATGACCAAGATGCGATA
GCGGTTGAGGCAGCTTGCACAAATGTCCCCGCCCTTTTCTTAGACGTGTCCGATCAAA
CGCCGATCAACTCCATTATCTTCTCTCACGAGGACGGCACTAGATTAGGCGTGGAACA
TCTTGTAGCGCTGGGGCACCAGCAAATCGCATTGCTAGCGGGACCGCTATCTTCCGTT
TCAGCTCGTCTAAGACTTGCGGGTTGGCATAAGTATTTGACAAGAAATCAAATTCAGC
CGATAGCCGAGCGTGAGGGGGATTGGTCAGCAATGTCAGGTTTCCAGCAGACGATGC
AGATGCTGAACGAAGGGATTGTCCCAACTGCCATGCTTGTCGCTAACGACCAGATGG
CGCTGGGCGCCATGCGTGCTATAACCGAGTCAGGCTTAAGGGTGGGCGCGGACATCT
CAGTTGTGGGATACGACGATACAGAGGATTCATCCTGTTACATACCGCCTTTGACCAC
GATTAAGCAAGACTTTCGTCTATTAGGACAGACGTCAGTAGACAGGTTATTGCAGCTG
AGTCAGGGACAGGCAGTCAAAGGTAACCAGCTTCTACCTGTCTCTCTAGTCAAGAGA
AAGACGACCCTGGCTCCGAATACGCAGACAGCCTCACCGCGTGCTCTTGCGGACTCA
CTTATGCAGCTGGCCAGGCAGGTTTCCAGACTAGAAAGCGGACAATGA
I⁺YQR L241A
SEQ ID NO: 53
ATGAAACCGGTAACGCTTTACGACGTAGCCGAATACGCCGGGGTATCCTACCAGACC
GTGAGTCGTGTCGTAAATCAGGCCTCCCATGTAAGCGCTAAGACAAGAGAGAAAGTG
GAAGCGGCTATGGCAGAGTTGAATTACATCCCCAACCGTGTCGCCCAACAGCTAGCG
GGTAAACAGTCATTACTTATCGGCGTTGCGACAAGCTCTTTGGCGTTACACGCCCCCA
GTCAGATAGTTGCAGCCATCAAGAGTAGGGCTGACCAGCTAGGCGCAAGTGTCGTAG
TGTCAATGGTTGAGAGATCAGGGGTCGAGGCTTGTAAGACGGCTGTGCATAACTTAC
TTGCCCAAAGAGTCTCTGGTTTAATAATAAATTACCCATTGGATGACCAAGATGCGATA
GCGGTTGAGGCAGCTTGCACAAATGTCCCCGCCCTTTTCTTAGACGTGTCCGATCAAA
CGCCGATCAACTCCATTATCTTCTCTCACGAGGACGGCACTAGATTAGGCGTGGAACA
TCTTGTAGCGCTGGGGCACCAGCAAATCGCATTGCTAGCGGGACCGCTATCTTCCGTT
TCAGCTCGTCTAAGACTTGCGGGTTGGCATAAGTATTTGACAAGAAATCAAATTCAGC
CGATAGCCGAGCGTGAGGGGGATTGGTCAGCAATGTCAGGTTTCCAGCAGACGATGC
AGATGCTGAACGAAGGGATTGTCCCAACTGCCATGCTTGTCGCTAACGACCAGATGG
CGGCGGGCGCCATGCGTGCTATAACCGAGTCAGGCTTAAGGGTGGGCGCGGACATCT
CAGTTGTGGGATACGACGATACAGAGGATTCATCCTGTTACATACCGCCTTTGACCAC
GATTAAGCAAGACTTTCGTCTATTAGGACAGACGTCAGTAGACAGGTTATTGCAGCTG
AGTCAGGGACAGGCAGTCAAAGGTAACCAGCTTCTACCTGTCTCTCTAGTCAAGAGA
AAGACGACCCTGGCTCCGAATACGCAGACAGCCTCACCGCGTGCTCTTGCGGACTCA
CTTATGCAGCTGGCCAGGCAGGTTTCCAGACTAGAAAGCGGACAATGA
IA(9)YQR
SEQ ID NO: 54
GTGAAACCAGTAACGTTATACGATGTCGCAGAGTATGCCGGTGTCTCTTATCAGACCG
TTTCCCGCGTGGTGAACCAGGCCAGCCACGTTTCTGCGAAAACGCGGGAAAAAGTG
GAAGCGGCGATGGCGGAGCTGAATTACATTCCCAACCGCGTGGCACAACAACTGGCG
GGCAAACAGTCGTTGCTGATTGGCGTTGCCACCTCCAGTCTGGCCCTGCACGCGCTG
TCGCAAATTGTCGCGGCGATTAAATCTCGCGCCTATCAACTGGGTGCCAGCGTGTTCG
TGTCGATGGTAGAACGAAGCGGCATCGAAGCCTGTAAAACGGCGGTGCACAATCTTC
TCGCGCAACGCGTCAGTGGGCTGATCATTAACTATCCGCTGGATAACCAGGATGCCAT
TGCTGTGGAAGCTGCCTGCACTAATGTTCCGGCGTTATTTCTTGATGTCTCTGACCAG
ACACCCATCAACAGTATTATTTTCTCCCATGAAGACGGTACGCGACTGGGCGTGGAGC
ATCTGGTCGCATTGGGTCACCAGCAAATCGCGCTGTTAGCGGGCCCATTAAGTTCTGT
CTCGGCGCGTCTGCGTCTGGCTGGCTGGCATAAATATCTCACTCGCAATCAAATTCAG
CCGATAGCGGAACGGGAAGGCGACTGGAGTGCCATGTCCGGTTTTCAACAAACCATG
CAAATGCTGAATGAGGGCATCGTTCCCACTGCGATGCTGGTTGCCAACGATCAGATG
GCGCTGGGCGCAATGCGCGCCATTACCGAGACCGGGCTGCGCGTTGGTGCGGATATC
TCGGTAGTGGGATACGACGATACCGAAGACAGCTCATGTTATATCCCGCCGTTAACCA
CCATCAAACAGGATTTTCGCCTGCTGGGGCAAACCAGCGTGGACCGCTTGCTGCAAC
TCTCTCAGGGCCAGGCGGTGAAGGGCAATCAGCTGTTGCCCGTCTCACTGGTGAAAA
GAAAAACCACCCTGGCGCCCAATACGCAAACCGCCTCTCCCCGCGCGTTGGCCGATT
CATTAATGCAGCTGGCACGACAGGTTTCCCGACTGGAAAGCGGGCAGTGA
IA(9)YQR L349*
SEQ ID NO: 55
ATGAAACCAGTAACGTTATACGATGTCGCAGAGTATGCCGGTGTCTCTTATCAGACCG
TTTCCCGCGTGGTGAACCAGGCCAGCCACGTTTCTGCGAAAACGCGGGAAAAAGTG
GAAGCGGCGATGGCGGAGCTGAATTACATTCCCAACCGCGTGGCACAACAACTGGCG
GGCAAACAGTCGTTGCTGATTGGCGTTGCCACCTCCAGTCTGGCCCTGCACGCGCTG
TCGCAAATTGTCGCGGCGATTAAATCTCGCGCCTATCAACTGGGTGCCAGCGTGTTCG
TGTCGATGGTAGAACGAAGCGGCATCGAAGCCTGTAAAACGGCGGTGCACAATCTTC
TCGCGCAACGCGTCAGTGGGCTGATCATTAACTATCCGCTGGATAACCAGGATGCCAT
TGCTGTGGAAGCTGCCTGCACTAATGTTCCGGCGTTATTTCTTGATGTCTCTGACCAG
ACACCCATCAACAGTATTATTTTCTCCCATGAAGACGGTACGCGACTGGGCGTGGAGC
ATCTGGTCGCATTGGGTCACCAGCAAATCGCGCTGTTAGCGGGCCCATTAAGTTCTGT
CTCGGCGCGTCTGCGTCTGGCTGGCTGGCATAAATATCTCACTCGCAATCAAATTCAG
CCGATAGCGGAACGGGAAGGCGACTGGAGTGCCATGTCCGGTTTTCAACAAACCATG
CAAATGCTGAATGAGGGCATCGTTCCCACTGCGATGCTGGTTGCCAACGATCAGATG
GCGCTGGGCGCAATGCGCGCCATTACCGAGACCGGGCTGCGCGTTGGTGCGGATATC
TCGGTAGTGGGATACGACGATACCGAAGACAGCTCATGTTATATCCCGCCGTTAACCA
CCATCAAACAGGATTTTCGCCTGCTGGGGCAAACCAGCGTGGACCGCTTGCTGCAAC
TCTCTCAGGGCCAGGCGGTGAAGGGCAATCAGCTGTTGCCaGTaTCACTGGTGAAAA
GAAAAACCACCCTGGCGCCCAATACGCAAACCGCCTCTCCCCGCGCGTTGGCCGATT
CATTAATGCAGTAA
R⁺YQR
SEQ ID NO: 56
ATGAAACCGGTAACTTTGTACGACGTAGCTGAATACGCTGGTGTATCTTACCAGACTG
TATCTCGTGTAGTAAACCAGGCTTCTCACGTATCTGCTAAAACTCGTGAAAAAGTAGA
AGCTGCTATGGCTGAATTGAACTACATCCCGAACCGTGTAGCTCAGCAGTTGGCTGGT
AAAGCTTCTCACACTATCGGTATGTTGATCACTGCTTCTACTAACCCGTTCTACTCTGA
ATTGGTACGTGGTGTAGAACGTTCTTGTTTCGAACGTGGTTACTCTTTGGTATTGTGTA
ACACTGAAGGTGACGAACAGCGTATGAACCGTAACTTGGAAACTTTGATGCAGAAAC
GTGTAGACGGTTTGTTGTTGTTGTGTACTGAAACTCACCAGCCGTCCCGTGAAATCAT
GCAGCGTTACCCGACTGTACCGACTGTAATGATGGACTGGGCTCCGTTCGACGGTGA
CTCTGACTTGATCCAGGACAACTCTTTGTTGGGTGGTGACTTGGCTACTCAGTACTTG
ATCGACAAAGGTCACACTCGTATCGCTTGTATCACTGGTCCGTTGGACAAAACTCCGG
CTCGTTTGCGTTTGGAAGGTTACCGTGCTGCTATGAAACGTGCTGGTTTGAACATCCC
GGACGGTTACGAAGTAACTGGTGACTTCGAATTCAACGGTGGTTTCGACGCTATGCG
TCAGTTGTTGTCTCACCCGTTGCGTCCGCAGGCTGTATTCACTGGTAACGACGCTATG
GCTGTAGGTGTATACCAGGCTTTGTACCAGGCTGAATTGCAGGTACCGCAGGACATCG
CTGTAATCGGTTACGACGACATCGAATTGGCTTCTTTCATGACTCCGCCGTTGACTACT
ATCCACCAGCCGAAAGACGAATTGGGTGAATTGGCTATCGACGTATTGATCCACCGTA
TCACTCAGCCGACTTTGCAGCAGCAGCGTTTGCAGTTGACTCCGATCTTGATGGAAC
GTGGTTCTGCTTAA
RA(2)YQR
SEQ ID NO: 57
GTGAAACCAGTAACGTTATACGATGTCGCAGAGTATGCCGGTGTCTCTTATCAGACCG
TTTCCCGCGTGGTGAACCAGGCCAGCCACGTTTCTGCGAAAACGCGGGAAAAAGTG
GAAGCGGCGATGGCGGAGCTCAATTACATTCCCAACCGCGTGGCACAACAACTGGCG
GGCAAAGCGTCGCATACCATTGGCATGTTGATCACTGCCAGTACCAATCCTTTCTATTC
AGAACTGGTGCGTGGCGTTGAACGCAGCTGCTTCGAACGCGGTTATAGTCTCGTCCC
TTGCAATACCGAAGGCGATGAACAGCGGATGAATCGCAATCTGGAAACGCTGATGCA
AAAACGCGTTGATGGCTTGCTGTTACTGTGCACCGAAACGCATCAACCTTCGCGTGA
AATCATGCAACGTTATCCGACAGTGCCTACTGTGATGATGGACTGGGCTCCGTTCGAT
GGCGACAGCGATCTTATTCAGGATAACTCGTTGCTGGGCGGAGACTTAGCAACGCAA
TATCTGATCGATAAAGGTCATACCCGTATCGCCTGTATTACCGGCCCGCTGGATAAAAC
TCCGGCGCGCCTGCGGTTGGAAGGTTATCGGGCGGCGATGAAACGTGCGGGTCTCAA
CATTCCTGATGGCTATGAAGTCACTGGTGATTTTGAATTTAACGGCGGGTTTGACGCT
ATGCGCCAACTGCTATCACATCCGCTGCGTCCTCAGGCCGTCTTTACCGGAAATGACG
CTATGGCTGTTGGCGTTTACCAGGCGTTATATCAGGCAGAGTTACAGGTTCCGCAGGA
TATCGCGGTGATTGGCTATGACGATATCGAACTGGCAAGCTTTATGACGCCACCATTAA
CCACTATCCACCAACCGAAAGATGAACTGGGGGAGCTGGCGATTGATGTACTCATCC
ATCGGATAACCCAGCCGACCCTTCAGCAACAACGATTACAACTTACTCCGATTCTGAT
GGAACGCGGTTCGGCTTAGATGAAACCAGTAACGTTATACGATGTCGCAGAGTATGCC
GGTGTCTCTACCGCGACCGTTTCCAACGTGGTGAACCAGGCCAGCCACGTTTCTGCG
AAAACGCGGGAAAAAGTGGAAGCGGCGATCAAAGAGCTGGGCTACGTGCCGAACC
GCGCAGCCCGCACCC
E⁺TAN
SEQ ID NO: 58
TGGTCACCCGACGTACCGACACCGTAGCCCTGGTGGTGTCGGAAAACAACCAGAAG
CTCTTCGCCGAACCCTTCTATGCCGGGATCGTGCTCGGCGTGGGGGTTGCTCTGTCCG
AACGGGGATTCCAGTTCGTCCTGGCCACGGGCCGCTCCGGGATAGAGCATGAGCGGC
TGGGCGGCTACCTGGCCGGACAGCACGTCGACGGGGTCCTCCTGCTGTCGCTCCACC
GCGACGACCCGCTGCCGCAGATGCTGGACGAGGCCGGGGTGCCGTACGTCTACGGC
GGCCGTCCGCTCGGCGTCCCCGAAGAACAGGTGTCCTATGTCGATATCGACAACATC
GGCGGGGGACGCCAGGCCACCCAGCGGCTGATCGAGACCGGGCACCGGCGGATCGC
TACGATCGCGGGCCCGCAGGACATGGTCGCTGGTGTGGAACGCCTCCAGGGGTATCG
CGAAGCACTGCTCGCCGCGGGGATGGAGTACGACGAAACACTGGTGAGCTACGGTG
ACTTCACCTACGACAGCGGGGTGGCCGCGATGCGGGAGCTGCTGGATCGGGCCCCCG
ACGTGGACGCCGTGTTCGCGGCCTCCGACTTGATGGGGCTGGCCGCGCTGCGGGTGC
TGCGTGCTTCGGGACGCCGCGTGCCCGAGGATGTGGCGGTGGTCGGCTACGACGACT
CGACCGTAGCCGAGCACGCCGAACCGCCGATGACCAGCGTCAACCAGCCCACCGAG
CTGATGGGCCGGGAGATGGCCCGGCTGCTCGTGGACCGGATCACCGGGGAGACCAC
CGAACCGGTGCGGCTGGTGCTGGAGACCCATTTGATGGTGCGGGAATCCGGGTGA
PhlFAM
SEQ ID NO: 59
ATGGCACGTACCCCGAGCCGTAGCAGCATTGGTAGCCTGCGTAGTCCGCATACCCATA
AAGCAATTCTGACCAGCACCATTGAAATCCTGAAAGAATGTGGTTATAGCGGTCTGA
GCATTGAAAGCGTGGCACGTCGCGCCGGTGCAGGCAAACCGACCATTTATCGTTGGT
GGACCAACAAAGCAGCACTGATTGCCGAAGTGTATGAAAATGAAATCGAACAGGTAC
GTAAATTTCCGGATTTGGGTAGCTTTAAAGCCGATCTGGATTTTCTGCTGCATAATCTG
TGGAAAGTTTGGCGTGAAACCATTTGTGGTGAAGCATTTCGTTGTGTTATTGCAGAAG
CACAGTTGGACCCTGTAACCCTGACCCAACTGAAAGATCAGTTTATGGAACGTCGTC
GTGAGATACCGAAAAAACTGGTTGAAGATGCCATTAGCAATGGTGAACTGCCGAAAG
ATATCAATCGTGAACTGCTGCTGGATATGATTTTTGGTTTTTGTTGGTATCGCCTGCTG
ACCGAACAGTTGACCGTTGAACAGGATATTGAAGAATTTACCTTCCTGCTGATTAATG
GTGTTTGTCCGGGTACACAGTGTTGA
A118 Recombinase
SEQ ID NO: 60
GTGAAGGCAGCTATTTATATACGTGTTTCTACTCAAGAGCAAGTAGAAAATTATTCAAT
ACAAGCTCAAACTGAAAAACTAACAGGATTGTGCCGCTCGAAGGACTGGGACGTATA
CGATATTTTCATTGACGGCGGATACTCCGGCTCAAATATGAATCGTCCCGCATTAAATG
AAATGCTAAGTAAACTACACGAAATTGATGCTGTAGTCGTATATCGATTAGACAGACT
ATCCCGCTCACAAAGAGACACAATAACGCTTATTGAAGAATACTTCTTAAAAAACAAT
GTAGAGTTTGTTAGTTTGTCTGAAACGCTTGATACTAGTTCCCCTTTCGGTCGTGCAAT
GATTGGTATATTATCAGTATTCGCACAGCTAGAGCGCGAAACAATCCGAGATCGTATGG
TGATGGGGAAAATTAAGCGTATTGAAGCAGGTCTTCCGTTAACAACTGCGAAAGGTA
GAACGTTCGGCTATGATGTTATAGATACAAAATTATACATTAATGAAGAAGAAGCAAA
ACAGTTACAACTGATTTATGATATTTTCGAAGAAGAACAAAGTATAACTTTTTTACAGA
AAAGACTAAAAAAATTAGGCTTTAAAGTTAGAACATATAATCGCTATAACAACTGGCT
AACTAATGATTTGTATTGTGGTTATGTTTCATATAAAGATAAAGTTCATGTAAAAGGTAT
TCATGAACCTATCATCAGTGAAGAGCAATTCTATAGAGTTCAAGAAATATTTACTCGTA
TGGGTAAAAATCCGAACATGAATAGAGATTCAGCATCGTTGCTAAATAATTTAGTAGTT
TGTAGTAAATGCGGGTTAGGCTTTGTTCATCGTAGAAAAGATACAATGTCGCGTGGTA
AAAAATATCATTATAGATATTATAGTTGCAAGACTTATAAACATACTCATGAACTCGAA
AAATGCGGGAATAAAATTTGGAGAGCTGACAAACTTGAAGAATTAATTATTAATCGTG
TGAATAATTATAGTTTCGCTTCCAGAAATGTAGATAAAGAAGATGAATTAGATAGCTTA
AATGAAAAGCTTAAAATAGAACATGCAAAGAAAAAACGATTATTTGATTTATATATAA
ATGGCTCGTATGAAGTTTCAGAACTTGATTCTATGATGAATGATATTGATGCTCAAATT
AATTATTATGAATCACAAATAGAAGCTAACGAAGAATTGAAGAAAAACAAAAAGATA
CAAGAAAATTTAGCTGATTTAGCAACAGTTGATTTTGACTCTTTAGAGTTCAGAGAAA
AGCAACTTTATTTAAAATCACTAATAAACAAAATTTATATTGATGGTGAACAAGTTACT
ATTGAATGGCTC
dCas9
SEQ ID NO: 61
ATGGATAAGAAATACTCAATAGGCTTAGCTATCGGCACAAATAGCGTCGGATGGGCGG
TGATCACTGATGAATATAAGGTTCCGTCTAAAAAGTTCAAGGTTCTGGGAAATACAGA
CCGCCACAGTATCAAAAAAAATCTTATAGGGGCTCTTTTATTTGACAGTGGAGAGACA
GCGGAAGCGACTCGTCTCAAACGGACAGCTCGTAGAAGGTATACACGTCGGAAGAAT
CGTATTTGTTATCTACAGGAGATTTTTTCAAATGAGATGGCGAAAGTAGATGATAGTTT
CTTTCATCGACTTGAAGAGTCTTTTTTGGTGGAAGAAGACAAGAAGCATGAACGTCA
TCCTATTTTTGGAAATATAGTAGATGAAGTTGCTTATCATGAGAAATATCCAACTATCTA
TCATCTGCGAAAAAAATTGGTAGATTCTACTGATAAAGCGGATTTGCGCTTAATCTATT
TGGCCTTAGCGCATATGATTAAGTTTCGTGGTCATTTTTTGATTGAGGGAGATTTAAAT
CCTGATAATAGTGATGTGGACAAACTATTTATCCAGTTGGTACAAACCTACAATCAATT
ATTTGAAGAAAACCCTATTAACGCAAGTGGAGTAGATGCTAAAGCGATTCTTTCTGCA
CGATTGAGTAAATCAAGACGATTAGAAAATCTCATTGCTCAGCTCCCCGGTGAGAAG
AAAAATGGCTTATTTGGGAATCTCATTGCTTTGTCATTGGGTTTGACCCCTAATTTTAA
ATCAAATTTTGATTTGGCAGAAGATGCTAAATTACAGCTTTCAAAAGATACTTACGATG
ATGATTTAGATAATTTATTGGCGCAAATTGGAGATCAATATGCTGATTTGTTTTTGGCAG
CTAAGAATTTATCAGATGCTATTTTACTTTCAGATATCCTAAGAGTAAATACTGAAATAA
CTAAGGCTCCCCTATCAGCTTCAATGATTAAACGCTACGATGAACATCATCAAGACTT
GACTCTTTTAAAAGCTTTAGTTCGACAACAACTTCCAGAAAAGTATAAAGAAATCTTT
TTTGATCAATCAAAAAACGGATATGCAGGTTATATTGATGGGGGAGCTAGCCAAGAAG
AATTTTATAAATTTATCAAACCAATTTTAGAAAAAATGGATGGTACTGAGGAATTATTG
GTGAAACTAAATCGTGAAGATTTGCTGCGCAAGCAACGGACCTTTGACAACGGCTCT
ATTCCCCATCAAATTCACTTGGGTGAGCTGCATGCTATTTTGAGAAGACAAGAAGACT
TTTATCCATTTTTAAAAGACAATCGTGAGAAGATTGAAAAAATCTTGACTTTTCGAATT
CCTTATTATGTTGGTCCATTGGCGCGTGGCAATAGTCGTTTTGCATGGATGACTCGGAA
GTCTGAAGAAACAATTACCCCATGGAATTTTGAAGAAGTTGTCGATAAAGGTGCTTC
AGCTCAATCATTTATTGAACGCATGACAAACTTTGATAAAAATCTTCCAAATGAAAAA
GTACTACCAAAACATAGTTTGCTTTATGAGTATTTTACGGTTTATAACGAATTGACAAA
GGTCAAATATGTTACTGAAGGAATGCGAAAACCAGCATTTCTTTCAGGTGAACAGAA
GAAAGCCATTGTTGATTTACTCTTCAAAACAAATCGAAAAGTAACCGTTAAGCAATTA
AAAGAAGATTATTTCAAAAAAATAGAATGTTTTGATAGTGTTGAAATTTCAGGAGTTG
AAGATAGATTTAATGCTTCATTAGGTACCTACCATGATTTGCTAAAAATTATTAAAGATA
AAGATTTTTTGGATAATGAAGAAAATGAAGATATCTTAGAGGATATTGTTTTAACATTG
ACCTTATTTGAAGATAGGGAGATGATTGAGGAAAGACTTAAAACATATGCTCACCTCT
TTGATGATAAGGTGATGAAACAGCTTAAACGTCGCCGTTATACTGGTTGGGGACGTTT
GTCTCGAAAATTGATTAATGGTATTAGGGATAAGCAATCTGGCAAAACAATATTAGATT
TTTTGAAATCAGATGGTTTTGCCAATCGCAATTTTATGCAGCTGATCCATGATGATAGT
TTGACATTTAAAGAAGACATTCAAAAAGCACAAGTGTCTGGACAAGGCGATAGTTTA
CATGAACATATTGCAAATTTAGCTGGTAGCCCTGCTATTAAAAAAGGTATTTTACAGAC
TGTAAAAGTTGTTGATGAATTGGTCAAAGTAATGGGGCGGCATAAGCCAGAAAATATC
GTTATTGAAATGGCACGTGAAAATCAGACAACTCAAAAGGGCCAGAAAAATTCGCGA
GAGCGTATGAAACGAATCGAAGAAGGTATCAAAGAATTAGGAAGTCAGATTCTTAAA
GAGCATCCTGTTGAAAATACTCAATTGCAAAATGAAAAGCTCTATCTCTATTATCTCCA
AAATGGAAGAGACATGTATGTGGACCAAGAATTAGATATTAATCGTTTAAGTGATTATG
ATGTCGATGCCATTGTTCCACAAAGTTTCCTTAAAGACGATTCAATAGACAATAAGGT
CTTAACGCGTTCTGATAAAAATCGTGGTAAATCGGATAACGTTCCAAGTGAAGAAGTA
GTCAAAAAGATGAAAAACTATTGGAGACAACTTCTAAACGCCAAGTTAATCACTCAA
CGTAAGTTTGATAATTTAACGAAAGCTGAACGTGGTGGTTTGAGTGAACTTGATAAAG
CTGGTTTTATCAAACGCCAATTGGTTGAAACTCGCCAAATCACTAAGCATGTGGCACA
AATTTTGGATAGTCGCATGAATACTAAATACGATGAAAATGATAAACTTATTCGAGAGG
TTAAAGTGATTACCTTAAAATCTAAATTAGTTTCTGACTTCCGAAAAGATTTCCAATTC
TATAAAGTACGTGAGATTAACAATTACCATCATGCCCATGATGCGTATCTAAATGCCGT
CGTTGGAACTGCTTTGATTAAGAAATATCCAAAACTTGAATCGGAGTTTGTCTATGGT
GATTATAAAGTTTATGATGTTCGTAAAATGATTGCTAAGTCTGAGCAAGAAATAGGCA
AAGCAACCGCAAAATATTTCTTTTACTCTAATATCATGAACTTCTTCAAAACAGAAATT
ACACTTGCAAATGGAGAGATTCGCAAACGCCCTCTAATCGAAACTAATGGGGAAACT
GGAGAAATTGTCTGGGATAAAGGGCGAGATTTTGCCACAGTGCGCAAAGTATTGTCC
ATGCCCCAAGTCAATATTGTCAAGAAAACAGAAGTACAGACAGGCGAATTCTCCAAG
GAGTCAATTTTACCAAAAAGAAATTCGGACAAGCTTATTGCTCGTAAAAAAGACTGG
GATCCAAAAAAATATGGTGGTTTTGATAGTCCAACGGTAGCTTATTCAGTCCTAGTGG
TTGCTAAGGTGGAAAAAGGGAAATCGAAGAAGTTAAAATCCGTTAAAGAGTTACTAG
GGATCACAATTATGGAAAGAAGTTCCTTTGAAAAAAATCCGATTGACTTTTTAGAAGC
TAAAGGATATAAGGAAGTTAAAAAAGACTTAATCATTAAACTACCTAAATATAGTCTTT
TTGAGTTAGAAAACGGTCGTAAACGGATGCTGGCTAGTGCCGGAGAATTACAAAAAG
GAAATGAGCTGGCTCTGCCAAGCAAATATGTGAATTTTTTATATTTAGCTAGTCATTAT
GAAAAGTTGAAGGGTAGTCCAGAAGATAACGAACAAAAACAATTGTTTGTGGAGCA
GCATAAGCATTATTTAGATGAGATTATTGAGCAAATCAGTGAATTTTCTAAGCGTGTTA
TTTTAGCAGATGCCAATTTAGATAAAGTTCTTAGTGCATATAACAAACATAGAGACAA
ACCAATACGTGAACAAGCAGAAAATATTATTCATTTATTTACGTTGACGAATCTTGGAG
CTCCCGCTGCTTTTAAATATTTTGATACAACAATTGATCGTAAACGATATACGTCTACA
AAAGAAGTTTTAGATGCCACTCTTATCCATCAATCCATCACTGGTCTTTATGAAACACG
CATTGATTTGAGTCAGCTAGGAGGTGACTGA
engRBS3 sgRNA (non-coding)
SEQ ID NO: 62
CTCATGAATACCTCCTTAAGGTTTTAGAGCTAGAAATAGCAAGTTAAAATAAGGCTAG
TCCGTTATCAACTTGAAAAAGTGGCACCGAGTCGGTGCTTTTTT
BBa_B0034 sgRNA (non-coding)
SEQ ID NO: 63
TGGTACCTTTCTCCTCTTTAGTTTTAGAGCTAGAAATAGCAAGTTAAAATAAGGCTAGT
CCGTTATCAACTTGAAAAAGTGGCACCGAGTCGGTGCTTTTTT
PBD(10)rep top
SEQ ID NO: 64
TTGCAAATTAATCATCCGGCTATTATAATTAGGACTGAGCTAGCTTGCAA
PBD(10)rep bottom
SEQ ID NO: 65
AACGTTTAATTAGTAGGCCGATAATATTAATCCTGACTCGATCGAACGTT
PBD(17)rep top
SEQ ID NO: 66
ATTATACCTAGGTTGCAATTAAATTGCAAGGCTCGTATAAT
PBD(17)rep bottom
SEQ ID NO: 67
TAATATGGATCCAACGTTAATTTAACGTTCCGAGCATATTA
PBD(35)rep bottom
SEQ ID NO: 68
TAATATGGATCCTGACTCGATCGAACGTTTAATTAGTAGGCCGAGCATATTA

What is claimed is:

1. A gene circuit capable of synthetic biological program operations comprising a quantum logic gate, wherein the quantum logic gate comprises at least one input, a bi-directional promoter, at least two synthetic transcription factors, and at least one cognate synthetic promoter.

2. The gene circuit of claim 1, wherein the synthetic biological program operation is a biological QUBIT or PAULI-X logic gate.

3. The gene circuit of claim 1, wherein one input results in two outputs.

4. The gene circuit of claim 1, wherein the input is isopropyl β-D-1-thiogalactopyranoside (IPTG), D-ribose, fructose, or cellobiose.

5. The gene circuit of claim 1, wherein the bi-directional promoter comprises SEQ ID NO: 9, SEQ ID NO: 64, SEQ ID NO: 66, or a sequence at least 90% identical to SEQ ID NO: 9, SEQ ID NO: 64, or SEQ ID NO: 66.

6. The gene circuit of claim 1, wherein the at least two synthetic transcription factors comprise a repressor and an anti-repressor.

7. The gene circuit of claim 1, wherein the gene circuit comprises at least one additional quantum logic gate layered to engineer additional synthetic biological program operations of increasing complexity.

8. The gene circuit of claim 7, wherein the synthetic biological program operation has a quantum-inspired programming logic gate equivalent to a FEYNMAN or TOFFOLI gate.

9. The gene circuit of claim 7, wherein two inputs result in four outputs.

10. The gene circuit of claim 9, wherein the inputs are selected from isopropyl β-D-1-thiogalactopyranoside (IPTG), D-ribose, fructose, and cellobiose.

11. The gene circuit of claim 9, wherein the gene circuit performs a swap-of-function memory operation, wherein the regulatory control of the output channels is swapped.

12. The gene circuit of claim 11, wherein the swap-of-function memory operation is performed by at least one recombinase.

13. A bi-directional promoter comprising SEQ ID NO: 9, SEQ ID NO: 64, SEQ ID NO: 66, or a sequence at least 90% identical to SEQ ID NO: 9, SEQ ID NO: 64, or SEQ ID NO: 66.

14. The bi-directional promoter of claim 13, wherein the bi-directional promoter reduces cellular resource competition.

15. The bi-directional promoter of claim 14, wherein the bi-directional promoter is oriented divergently with a single −35 hexamer.

16. A cell comprising a plurality of nucleic acid sequences, encoding a gene circuit comprising a bi-directional promoter, at least two synthetic transcription factors responsive to input, at least two cognate synthetic promoters, and at least two outputs.

17. The cell of claim 14, wherein the cell is a bacterial cell.

18. The cell of claim 14, wherein the plurality of nucleic acid sequences are operably linked.

19. The cell of claim 14, wherein the bi-directional promoter comprises SEQ ID NO: 9, SEQ ID NO: 64, SEQ ID NO: 66, or a sequence at least 90% identical to SEQ ID NO: 9, SEQ ID NO: 64, or SEQ ID NO: 66.

20. The cell of claim 14, wherein the at least two synthetic transcription factors responsive to input are a repressor and an anti-repressor.


SEQUENCE LISTING (68 sequences)

SEQ ID NO: 1 - DNA, 29 bp, Escherichia coli
  misc_feature 1..29: note = Description of sequence: oligonucleotide, name: PlacI
  source 1..29: mol_type = genomic DNA
gcgcaaaacctttcgcggtatggcatgat

SEQ ID NO: 2 - DNA, 29 bp, synthetic construct
  misc_feature 1..29: note = Description of sequence: oligonucleotide, name: PlacIQ
  source 1..29: mol_type = other DNA
gtgcaaaacctttcgcggtatggcatgat

SEQ ID NO: 3 - DNA, 29 bp, synthetic construct
  misc_feature 1..29: note = Description of sequence: oligonucleotide, name: PRA2HQN
  source 1..29: mol_type = other DNA
cgccaaaacctttcgcggtatggcatgat

SEQ ID NO: 4 - DNA, 29 bp, synthetic construct
  misc_feature 1..29: note = Description of sequence: oligonucleotide, name: Pta
  source 1..29: mol_type = other DNA
ttgacattttaagcgcttaaaattataat

SEQ ID NO: 5 - DNA, 29 bp, synthetic construct
  misc_feature 1..29: note = Description of sequence: oligonucleotide, name: PmJ23119
  source 1..29: mol_type = other DNA
tgtacagctagctcagtcctaggtataat

SEQ ID NO: 6 - DNA, 30 bp, synthetic construct
  misc_feature 1..30: note = Description of sequence: polypeptide, name: PTrc
  source 1..30: mol_type = other DNA
ttgacaattaatcatccggctcgtataatg

SEQ ID NO: 7 - DNA, 35 bp, synthetic construct
  misc_feature 1..35: note = Description of sequence: polypeptide, name: PPhlF
  source 1..35: mol_type = other DNA
ttgacatgatacgaaacgtaccgtatcgttaaggt

SEQ ID NO: 8 - DNA, 29 bp, synthetic construct
  misc_feature 1..29: note = Description of sequence: oligonucleotide, name: PdCas9
  source 1..29: mol_type = other DNA
ttgacagatagttgcatctgtgctataat

SEQ ID NO: 9 - DNA, 52 bp, synthetic construct
  misc_feature 1..52: note = Description of sequence: polypeptide, name: PBD(35)rep
  source 1..52: mol_type = other DNA
attatacctaggactgagctagcttgcaaattaatcatccggctcgtataat

SEQ ID NO: 10 - DNA, 52 bp, synthetic construct
  misc_feature 1..52: note = Description of sequence: polypeptide, name: PBD(35)lac0
  source 1..52: mol_type = other DNA
atcatgccataccgcgaaaggtttgtacaaacctttcgcggtatggcatgat

SEQ ID NO: 11 - DNA, 52 bp, synthetic construct
  misc_feature 1..52: note = Description of sequence: polypeptide, name: PBD(35)lac1
  source 1..52: mol_type = other DNA
atcatgccatatactgaaaggtttgtacaaacctttcgcggtatggcatgat

SEQ ID NO: 12 - DNA, 52 bp, synthetic construct
  misc_feature 1..52: note = Description of sequence: polypeptide, name: PBD(35)lac2
  source 1..52: mol_type = other DNA
atcatgccataaaattaaaggtttgtacaaacctttcgcggtatggcatgat

SEQ ID NO: 13 - DNA, 20 bp, synthetic construct
  misc_feature 1..20: note = Description of sequence: oligonucleotide, name: OSYM
  source 1..20: mol_type = other DNA
aattgtgagcgctcacaatt

SEQ ID NO: 14 - DNA, 20 bp, synthetic construct
  misc_feature 1..20: note = Description of sequence: oligonucleotide, name: Oagg
  source 1..20: mol_type = other DNA
aattaggagcgctcctaatt

SEQ ID NO: 15 - DNA, 20 bp, synthetic construct
  misc_feature 1..20: note = Description of sequence: oligonucleotide, name: Ottg
  source 1..20: mol_type = other DNA
aattttgagcgctcaaaatt

SEQ ID NO: 16 - DNA, 20 bp, synthetic construct
  misc_feature 1..20: note = Description of sequence: oligonucleotide, name: Otta
  source 1..20: mol_type = other DNA
aattttaagcgcttaaaatt

SEQ ID NO: 17 - DNA, 20 bp, synthetic construct
  misc_feature 1..20: note = Description of sequence: oligonucleotide, name: Ogta
  source 1..20: mol_type = other DNA
aattgtaagcgcttacaatt

SEQ ID NO: 18 - DNA, 28 bp, Escherichia coli
  misc_feature 1..28: note = Description of sequence: oligonucleotide, name: lac
  source 1..28: mol_type = genomic DNA
ggaagagagtcaattcagggtggtgaat

SEQ ID NO: 19 - DNA, 12 bp, synthetic construct
  misc_feature 1..12: note = Description of sequence: oligonucleotide, name: BBa_B0034
  source 1..12: mol_type = other DNA
aaagaggagaaa

SEQ ID NO: 20 - DNA, 25 bp, synthetic construct
  misc_feature 1..25: note = Description of sequence: oligonucleotide, name: C12
  source 1..25: mol_type = other DNA
gctgggaaggaatacgtaagaatta

SEQ ID NO: 21 - DNA, 28 bp, synthetic construct
  misc_feature 1..28: note = Description of sequence: oligonucleotide, name: ph12
  source 1..28: mol_type = other DNA
ggaagagagtcaattcatgggggtgaat

SEQ ID NO: 22 - DNA, 28 bp, synthetic construct
  misc_feature 1..28: note = Description of sequence: oligonucleotide, name: H8
  source 1..28: mol_type = other DNA
ggaagagagtcaattcatgggggtgaat

SEQ ID NO: 23 - DNA, 50 bp, synthetic construct
  misc_feature 1..50: note = Description of sequence: polypeptide, name: E21
  source 1..50: mol_type = other DNA
gctgggaaggaatccgtatgaattagctgggaaggaatgcgtaagaatta

SEQ ID NO: 24 - DNA, 25 bp, synthetic construct
  misc_feature 1..25: note = Description of sequence: oligonucleotide, name: F9
  source 1..25: mol_type = other DNA
cctgggaaggaatgcgtaggaatta

SEQ ID NO: 25 - DNA, 26 bp, synthetic construct
  misc_feature 1..26: note = Description of sequence: oligonucleotide, name: a118
  source 1..26: mol_type = other DNA
caaccatcgtattgaaggtggctaac

SEQ ID NO: 26 - DNA, 30 bp, synthetic construct
  misc_feature 1..30: note = Description of sequence: polypeptide, name: prgJ
  source 1..30: mol_type = other DNA
ctttaatttaacgtaaataaggaagtcatt

SEQ ID NO: 27 - DNA, 22 bp, synthetic construct
  misc_feature 1..22: note = Description of sequence: oligonucleotide, name: eng1
  source 1..22: mol_type = other DNA
aacaaaatgaggaggtactgag

SEQ ID NO: 28 - DNA, 19 bp, synthetic construct
  misc_feature 1..19: note = Description of sequence: oligonucleotide, name: eng3
  source 1..19: mol_type = other DNA
acaacttaaggaggtattc

SEQ ID NO: 29 - DNA, 17 bp, synthetic construct
  misc_feature 1..17: note = Description of sequence: oligonucleotide, name: eng5
  source 1..17: mol_type = other DNA
ctggtgaggaggtgaga

SEQ ID NO: 30 - DNA, 19 bp, synthetic construct
  misc_feature 1..19: note = Description of sequence: oligonucleotide, name: eng6
  source 1..19: mol_type = other DNA
aaagtaaggaggcgcggct

SEQ ID NO: 31 - DNA, 75 bp, synthetic construct
  misc_feature 1..75: note = Description of sequence: polypeptide, name: RiboJ
  source 1..75: mol_type = other DNA
agctgtcaccggatgtgctttccggtctgatgagtccgtgaggacgaaacagcctctacaaataattttgtttaa

SEQ ID NO: 32 - DNA, 75 bp, synthetic construct
  misc_feature 1..75: note = Description of sequence: polypeptide, name: RiboJ10
  source 1..75: mol_type = other DNA
agcgctcaacgggtgtgcttcccgttctgatgagtccgtgaggacgaaagcgcctctacaaataattttgtttaa

SEQ ID NO: 33 - DNA, 78 bp, synthetic construct
  misc_feature 1..78: note = Description of sequence: polypeptide, name: AraJ
  source 1..78: mol_type = other DNA
agtggtcgtgatctgaaactcgatcacctgatgagctcaaggcagagcgaaaccacctctacaaataattttgtttaa

SEQ ID NO: 34 - DNA, 76 bp, synthetic construct
  misc_feature 1..76: note = Description of sequence: polypeptide, name: BydvJ
  source 1..76: mol_type = other DNA
agggtgtctcaaggtgcgtaccttgactgatgagtccgaaaggacgaaacacccctctacaaataattttgtttaa

SEQ ID NO: 35 - DNA, 78 bp, synthetic construct
  misc_feature 1..78: note = Description of sequence: polypeptide, name: ElvJ
  source 1..78: mol_type = other DNA
agccccatagggtggtgtgtaccacccctgatgagtccaaaaggacgaaatggggcctctacaaataattttgtttaa

SEQ ID NO: 36 - DNA, 81 bp, synthetic construct
  misc_feature 1..81: note = Description of sequence: polypeptide, name: PlmJ
  source 1..81: mol_type = other DNA
agtcataagtctgggctaagcccactgatgagtcgctgaaatgcgacgaaacttatgacctctacaaataattttgtttaa

SEQ ID NO: 37 - DNA, 108 bp, synthetic construct
  misc_feature 1..108: note = Description of sequence: polypeptide, name: CchJ
  source 1..108: mol_type = other DNA
agttccagtcgagacctgaagtgggtttcctgatgaggctgtggagagagcgaaagctttactcccgcacaagccgaaactggaacctctacaaataattttgtttaa

SEQ ID NO: 38 - DNA, 43 bp, synthetic construct
  misc_feature 1..43: note = Description of sequence: polypeptide, name: HH1
  source 1..43: mol_type = other DNA
catgagctgatgagtccgtgaggacgaaacgagtaagctcgtc

SEQ ID NO: 39 - DNA, 39 bp, synthetic construct
  misc_feature 1..39: note = Description of sequence: polypeptide, name: HH2
  source 1..39: mol_type = other DNA
gcagtaagctgcctgaagagctgagtgcagcgaaaaaaa

SEQ ID NO: 40 - DNA, 43 bp, synthetic construct
  misc_feature 1..43: note = Description of sequence: polypeptide, name: HH3
  source 1..43: mol_type = other DNA
gtaccactgatgagtccgtgaggacgaaacgagtaagctcgtc

SEQ ID NO: 41 - DNA, 68 bp, synthetic construct
  misc_feature 1..68: note = Description of sequence: polypeptide, name: HDV
  source 1..68: mol_type = other DNA
ggccggcatggtcccagcctcctcgctggcgccggctgggcaacatgcttcggcatggcgaatgggac

SEQ ID NO: 42 - DNA, 72 bp, Escherichia coli
  misc_feature 1..72: note = Description of sequence: polypeptide, name: rrnBT1
  source 1..72: mol_type = genomic DNA
caaataaaacgaaaggctcagtcgaaagactgggcctttcgttttatctgttgtttgtcggtgaacgctctc

SEQ ID NO: 43 - DNA, 57 bp, synthetic construct
  misc_feature 1..57: note = Description of sequence: polypeptide, name: L3S2P11
  source 1..57: mol_type = other DNA
ctcggtaccaaattccagaaaagagacgctttcgagcgtcttttttcgttttggtcc

SEQ ID NO: 44 - DNA, 90 bp, synthetic construct
  misc_feature 1..90: note = Description of sequence: polypeptide, name: ECK120029600
  source 1..90: mol_type = other DNA
ttcagccaaaaaacttaagaccgccggtcttgtccactaccttgcagtaatgcggtggacaggatcggcggttttcttttctcttctcaa

SEQ ID NO: 45 - DNA, 57 bp, synthetic construct
  misc_feature 1..57: note = Description of sequence: polypeptide, name: ECK120033737
  source 1..57: mol_type = other DNA
ggaaacacagaaaaaagcccgcacctgacagtgcgggctttttttttcgaccaaagg

SEQ ID NO: 46 - DNA, 53 bp, synthetic construct
  misc_feature 1..53: note = Description of sequence: polypeptide, name: ECK120033736
  source 1..53: mol_type = other DNA
aacgcatgagaaagcccccggaagatcaccttccgggggcttttttattgcgc

SEQ ID NO: 47 - DNA, 54 bp, synthetic construct
  misc_feature 1..54: note = Description of sequence: polypeptide, name: ECK120010818
  source 1..54: mol_type = other DNA
gtcagtttcacctgttttacgtaaaaacccgcttcggcgggtttttacttttgg

SEQ ID NO: 48 - DNA, 714 bp, synthetic construct
  misc_feature 1..714: note = Description of sequence: polypeptide, name: sfGFP (GFP)
  source 1..714: mol_type = other DNA
atgagcaaaggagaagaacttttcactggagttgtcccaattcttgttgaattagatggtgatgttaatgggcacaaattttctgtccgtggagagggtgaaggtgatgctacaaacggaaagcttacccttaaatttatttgcactactggaaaactacctgttccatggccaacacttgtcactactctcacttatggtgttcaatgcttttcccgttatccggatcatatgaaacggcatgactttttcaagagtgccatgcccgaaggttatgtacaggaacgcactatatctttcaaagatgacgggacttacaagacgcgtgctgaagtcaagtttgaaggtgatacccttgttaatcgtatcgagttaaaaggtattgattttaaagaagatggaaacattctcggacacaaacttgagtacaactttaactcacacaatgtatacatcacggcagacaaacaaaagaatggaatcaaagctaacttcaaaattcgccacaacgttgaagatggatccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaa

SEQ ID NO: 49 - DNA, 711 bp, synthetic construct
  misc_feature 1..711: note = Description of sequence: polypeptide, name: mCherry (RFP)
  source 1..711: mol_type = other DNA
atggttagcaagggcgaggaagacaacatggcgatcatcaaagagttcatgcgttttaaggttcacatggaaggttctgttaacggtcacgagttcgaaatcgaaggtgaaggcgaaggccgtccgtacgaaggtacccagaccgcgaaactcaaggttaccaaaggtggtccgctgccgttcgcgtgggacatcctctctccgcaattcatgtacggttctaaagcgtacgttaaacatccggcagatatcccggactacctgaaactgtctttcccggaaggcttcaaatgggaacgtgttatgaactttgaagacggcggtgttgttaccgttacccaggactcttctctgcaggacggtgaatttatctataaggttaaactccgtggtactaacttcccgtctgacggtccggttatgcagaaaaagaccatgggttgggaagcgtcttctgaacgtatgtacccggaggatggtgcgctgaaaggcgagatcaaacagcgtctcaaactgaaagacggtggtcactacgacgcggaagtaaaaaccacctacaaagctaaaaagccagttcagctgccgggtgcgtacaacgtgaacattaaactggacatcacctctcacaacgaagactacaccattgttgaacagtacgaacgtgcggagggtcgtcactctaccggtggtatggacgaactgtacaaataa

SEQ ID NO: 50 - DNA, 693 bp, synthetic construct
  misc_feature 1..693: note = Description of sequence: polypeptide, name: tagBFP2 (BFP)
  source 1..693: mol_type = other DNA
atgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcactaa

SEQ ID NO: 51 - DNA, 705 bp, synthetic construct
  misc_feature 1..705: note = Description of sequence: polypeptide, name: phiYFP (YFP)
  source 1..705: mol_type = other DNA
atgagctccggcgcgctgctgtttcacggtaagatcccgtacgtggttgagatggagggcaacgttgacggtcataccttctccatccgtggtaaaggttatggcgacgctagcgtaggtaaagtggacgcgcagttcatctgcactactggtgacgtgccggttccgtggtctactctggtgactaccctgacctatggtgcccagtgcttcgccaaatacggtccagaactgaaagatttctacaaaagctgcatgcctgaaggctatgttcaggaacgtaccattaccttcgaaggcgacggtgtgttcaaaacccgcgcggaagtaacctttgaaaacggctccgtttacaaccgcgtgaaactgaacggtcaaggcttcaaaaaagacggtcacgtgctgggcaagaacctggaattcaactttaccccgcactgcctgtacatctggggtgaccaggcgaaccatggcctgaaatctgcgttcaagatcatgcacgagatcactggctctaaggaagactttatcgtggccgaccacactcagatgaacacgccaattggcggtggtccggttcacgtgccggaatatcaccacatcacgtaccacgttaccctgtccaaagacgtgactgatcaccgtgacaacatgtctctggttgaaactgttcgtgcggtggattgccgtaaaacgtacctgtaa

SEQ ID NO: 52 - DNA, 1083 bp, synthetic construct
  misc_feature 1..1083: note = Description of sequence: polypeptide, name: I+YQR
  source 1..1083: mol_type = other DNA
atgaaaccggtaacgctttacgacgtagccgaatacgccggggtatcctaccagaccgtgagtcgtgtcgtaaatcaggcctcccatgtaagcgctaagacaagagagaaagtggaagcggctatggcagagttgaattacatccccaaccgtgtcgcccaacagctagcgggtaaacagtcattacttatcggcgttgcgacaagctctttggcgttacacgcccccagtcagatagttgcagccatcaagagtagggctgaccagctaggcgcaagtgtcgtagtgtcaatggttgagagatcaggggtcgaggcttgtaagacggctgtgcataacttacttgcccaaagagtctctggtttaataataaattacccattggatgaccaagatgcgatagcggttgaggcagcttgcacaaatgtccccgcccttttcttagacgtgtccgatcaaacgccgatcaactccattatcttctctcacgaggacggcactagattaggcgtggaacatcttgtagcgctggggcaccagcaaatcgcattgctagcgggaccgctatcttccgtttcagctcgtctaagacttgcgggttggcataagtatttgacaagaaatcaaattcagccgatagccgagcgtgagggggattggtcagcaatgtcaggtttccagcagacgatgcagatgctgaacgaagggattgtcccaactgccatgcttgtcgctaacgaccagatggcgctgggcgccatgcgtgctataaccgagtcaggcttaagggtgggcgcggacatctcagttgtgggatacgacgatacagaggattcatcctgttacataccgcctttgaccacgattaagcaagactttcgtctattaggacagacgtcagtagacaggttattgcagctgagtcagggacaggcagtcaaaggtaaccagcttctacctgtctctctagtcaagagaaagacgaccctggctccgaatacgcagacagcctcaccgcgtgctcttgcggactcacttatgcagctggccaggcaggtttccagactagaaagcggacaatga

SEQ ID NO: 53 - DNA, 1083 bp, synthetic construct
  misc_feature 1..1083: note = Description of sequence: polypeptide, name: I+YQR L241A
  source 1..1083: mol_type = other DNA
atgaaaccggtaacgctttacgacgtagccgaatacgccggggtatcctaccagaccgtgagtcgtgtcgtaaatcaggcctcccatgtaagcgctaagacaagagagaaagtggaagcggctatggcagagttgaattacatccccaaccgtgtcgcccaacagctagcgggtaaacagtcattacttatcggcgttgcgacaagctctttggcgttacacgcccccagtcagatagttgcagccatcaagagtagggctgaccagctaggcgcaagtgtcgtagtgtcaatggttgagagatcaggggtcgaggcttgtaagacggctgtgcataacttacttgcccaaagagtctctggtttaataataaattacccattggatgaccaagatgcgatagcggttgaggcagcttgcacaaatgtccccgcccttttcttagacgtgtccgatcaaacgccgatcaactccattatcttctctcacgaggacggcactagattaggcgtggaacatcttgtagcgctggggcaccagcaaatcgcattgctagcgggaccgctatcttccgtttcagctcgtctaagacttgcgggttggcataagtatttgacaagaaatcaaattcagccgatagccgagcgtgagggggattggtcagcaatgtcaggtttccagcagacgatgcagatgctgaacgaagggattgtcccaactgccatgcttgtcgctaacgaccagatggcggcgggcgccatgcgtgctataaccgagtcaggcttaagggtgggcgcggacatctcagttgtgggatacgacgatacagaggattcatcctgttacataccgcctttgaccacgattaagcaagactttcgtctattaggacagacgtcagtagacaggttattgcagctgagtcagggacaggcagtcaaaggtaaccagcttctacctgtctctctagtcaagagaaagacgaccctggctccgaatacgcagacagcctcaccgcgtgctcttgcggactcacttatgcagctggccaggcaggtttccagactagaaagcggacaatga

SEQ ID NO: 54 - DNA, 1083 bp, synthetic construct
  misc_feature 1..1083: note = Description of sequence: polypeptide, name: IA(9)YQR
  source 1..1083: mol_type = other DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgctgtcgcaaattgtcgcggcgattaaatctcgcgcctatcaactgggtgccagcgtgttcgtgtcgatggtagaacgaagcggcatcgaagcctgtaaaacggcggtgcacaatcttctcgcgcaacgcgtcagtgggctgatcattaactatccgctggataaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagaccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 55 - DNA, 1047 bp, synthetic construct
  misc_feature 1..1047: note = Description of sequence: polypeptide, name: IA(9)YQR L349*
  source 1..1047: mol_type = other DNA
atgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgctgtcgcaaattgtcgcggcgattaaatctcgcgcctatcaactgggtgccagcgtgttcgtgtcgatggtagaacgaagcggcatcgaagcctgtaaaacggcggtgcacaatcttctcgcgcaacgcgtcagtgggctgatcattaactatccgctggataaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagaccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgccagtatcactggtgaaaagaaaaaccaccctggcgcccaatacgcaaaccgcctctccccgcgcgttggccgattcattaatgcagtaa

SEQ ID NO: 56 - DNA, 999 bp, synthetic construct
  misc_feature 1..999: note = Description of sequence: polypeptide, name: R+YQR
  source 1..999: mol_type = other DNA
atgaaaccggtaactttgtacgacgtagctgaatacgctggtgtatcttaccagactgtatctcgtgtagtaaaccaggcttctcacgtatctgctaaaactcgtgaaaaagtagaagctgctatggctgaattgaactacatcccgaaccgtgtagctcagcagttggctggtaaagcttctcacactatcggtatgttgatcactgcttctactaacccgttctactctgaattggtacgtggtgtagaacgttcttgtttcgaacgtggttactctttggtattgtgtaacactgaaggtgacgaacagcgtatgaaccgtaacttggaaactttgatgcagaaacgtgtagacggtttgttgttgttgtgtactgaaactcaccagccgtcccgtgaaatcatgcagcgttacccgactgtaccgactgtaatgatggactgggctccgttcgacggtgactctgacttgatccaggacaactctttgttgggtggtgacttggctactcagtacttgatcgacaaaggtcacactcgtatcgcttgtatcactggtccgttggacaaaactccggctcgtttgcgtttggaaggttaccgtgctgctatgaaacgtgctggtttgaacatcccggacggttacgaagtaactggtgacttcgaattcaacggtggtttcgacgctatgcgtcagttgttgtctcacccgttgcgtccgcaggctgtattcactggtaacgacgctatggctgtaggtgtataccaggctttgtaccaggctgaattgcaggtaccgcaggacatcgctgtaatcggttacgacgacatcgaattggcttctttcatgactccgccgttgactactatccaccagccgaaagacgaattgggtgaattggctatcgacgtattgatccaccgtatcactcagccgactttgcagcagcagcgtttgcagttgactccgatcttgatggaacgtggttctgcttaa

SEQ ID NO: 57 - DNA, 1165 bp, synthetic construct
  misc_feature 1..1165: note = Description of sequence: polypeptide, name: RA(2)YQR
  source 1..1165: mol_type = other DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctcaattacattcccaaccgcgtggcacaacaactggcgggcaaagcgtcgcataccattggcatgttgatcactgccagtaccaatcctttctattcagaactggtgcgtggcgttgaacgcagctgcttcgaacgcggttatagtctcgtcccttgcaataccgaaggcgatgaacagcggatgaatcgcaatctggaaacgctgatgcaaaaacgcgttgatggcttgctgttactgtgcaccgaaacgcatcaaccttcgcgtgaaatcatgcaacgttatccgacagtgcctactgtgatgatggactgggctccgttcgatggcgacagcgatcttattcaggataactcgttgctgggcggagacttagcaacgcaatatctgatcgataaaggtcatacccgtatcgcctgtattaccggcccgctggataaaactccggcgcgcctgcggttggaaggttatcgggcggcgatgaaacgtgcgggtctcaacattcctgatggctatgaagtcactggtgattttgaatttaacggcgggtttgacgctatgcgccaactgctatcacatccgctgcgtcctcaggccgtctttaccggaaatgacgctatggctgttggcgtttaccaggcgttatatcaggcagagttacaggttccgcaggatatcgcggtgattggctatgacgatatcgaactggcaagctttatgacgccaccattaaccactatccaccaaccgaaagatgaactgggggagctggcgattgatgtactcatccatcggataacccagccgacccttcagcaacaacgattacaacttactccgattctgatggaacgcggttcggcttagatgaaaccagtaacgttatacgatgtcgcagagtatgccggtgtctctaccgcgaccgtttccaacgtggtgaaccaggccagccacgtttctgcgaaaacgcgggaaaaagtggaagcggcgatcaaagagctgggctacgtgccgaaccgcgcagcccgcaccc

SEQ ID NO: 58 - DNA, 848 bp, synthetic construct
  misc_feature 1..848: note = Description of sequence: polypeptide, name: E+TAN
  source 1..848: mol_type = other DNA
tggtcacccgacgtaccgacaccgtagccctggtggtgtcggaaaacaaccagaagctcttcgccgaacccttctatgccgggatcgtgctcggcgtgggggttgctctgtccgaacggggattccagttcgtcctggccacgggccgctccgggatagagcatgagcggctgggcggctacctggccggacagcacgtcgacggggtcctcctgctgtcgctccaccgcgacgacccgctgccgcagatgctggacgaggccggggtgccgtacgtctacggcggccgtccgctcggcgtccccgaagaacaggtgtcctatgtcgatatcgacaacatcggcgggggacgccaggccacccagcggctgatcgagaccgggcaccggcggatcgctacgatcgcgggcccgcaggacatggtcgctggtgtggaacgcctccaggggtatcgcgaagcactgctcgccgcggggatggagtacgacgaaacactggtgagctacggtgacttcacctacgacagcggggtggccgcgatgcgggagctgctggatcgggcccccgacgtggacgccgtgttcgcggcctccgacttgatggggctggccgcgctgcgggtgctgcgtgcttcgggacgccgcgtgcccgaggatgtggcggtggtcggctacgacgactcgaccgtagccgagcacgccgaaccgccgatgaccagcgtcaaccagcccaccgagctgatgggccgggagatggcccggctgctcgtggaccggatcaccggggagaccaccgaaccggtgcggctggtgctggagacccatttgatggtgcgggaatccgggtga

SEQ ID NO: 59 - DNA, 603 bp, synthetic construct
  misc_feature 1..603: note = Description of sequence: polypeptide, name: PhlFAM
  source 1..603: mol_type = other DNA
atggcacgtaccccgagccgtagcagcattggtagcctgcgtagtccgcatacccataaagcaattctgaccagcaccattgaaatcctgaaagaatgtggttatagcggtctgagcattgaaagcgtggcacgtcgcgccggtgcaggcaaaccgaccatttatcgttggtggaccaacaaagcagcactgattgccgaagtgtatgaaaatgaaatcgaacaggtacgtaaatttccggatttgggtagctttaaagccgatctggattttctgctgcataatctgtggaaagtttggcgtgaaaccatttgtggtgaagcatttcgttgtgttattgcagaagcacagttggaccctgtaaccctgacccaactgaaagatcagtttatggaacgtcgtcgtgagataccgaaaaaactggttgaagatgccattagcaatggtgaactgccgaaagatatcaatcgtgaactgctgctggatatgatttttggtttttgttggtatcgcctgctgaccgaacagttgaccgttgaacaggatattgaagaatttaccttcctgctgattaatggtgtttgtccgggtacacagtgttga

SEQ ID NO: 60 - DNA, 1356 bp, synthetic construct
  misc_feature 1..1356: note = Description of sequence: polypeptide, name: A118 Recombinase
  source 1..1356: mol_type = other DNA
gtgaaggcagctatttatatacgtgtttctactcaagagcaagtagaaaattattcaatacaagctcaaactgaaaaactaacaggattgtgccgctcgaaggactgggacgtatacgatattttcattgacggcggatactccggctcaaatatgaatcgtcccgcattaaatgaaatgctaagtaaactacacgaaattgatgctgtagtcgtatatcgattagacagactatcccgctcacaaagagacacaataacgcttattgaagaatacttcttaaaaaacaatgtagagtttgttagtttgtctgaaacgcttgatactagttcccctttcggtcgtgcaatgattggtatattatcagtattcgcacagctagagcgcgaaacaatccgagatcgtatggtgatggggaaaattaagcgtattgaagcaggtcttccgttaacaactgcgaaaggtagaacgttcggctatgatgttatagatacaaaattatacattaatgaagaagaagcaaaacagttacaactgatttatgatattttcgaagaagaacaaagtataacttttttacagaaaagactaaaaaaattaggctttaaagttagaacatataatcgctataacaactggctaactaatgatttgtattgtggttatgtttcatataaagataaagttcatgtaaaaggtattcatgaacctatcatcagtgaagagcaattctatagagttcaagaaatatttactcgtatgggtaaaaatccgaacatgaatagagattcagcatcgttgctaaataatttagtagtttgtagtaaatgcgggttaggctttgttcatcgtagaaaagatacaatgtcgcgtggtaaaaaatatcattatagatattatagttgcaagacttataaacatactcatgaactcgaaaaatgcgggaataaaatttggagagctgacaaacttgaagaattaattattaatcgtgtgaataattatagtttcgcttccagaaatgtagataaagaagatgaattagatagcttaaatgaaaagcttaaaatagaacatgcaaagaaaaaacgattatttgatttatatataaatggctcgtatgaagtttcagaacttgattctatgatgaatgatattgatgctcaaattaattattatgaatcacaaatagaagctaacgaagaattgaagaaaaacaaaaagatacaagaaaatttagctgatttagcaacagttgattttgactctttagagttcagagaaaagcaactttatttaaaatcactaataaacaaaatttatattgatggtgaacaagttactattgaatggctc

SEQ ID NO: 61 - DNA, 4107 bp, synthetic construct
  misc_feature 1..4107: note = Description of sequence: polypeptide, name: dCas9
  source 1..4107: mol_type = other DNA
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggtggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcgaattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 62 - DNA, 102 bp, synthetic construct
  misc_feature 1..102: note = Description of sequence: polypeptide, name: engRBS3 sgRNA (non-coding)
  source 1..102: mol_type = other DNA
ctcatgaatacctccttaaggttttagagctagaaatagcaagttaaaataaggctagtccgttatcaacttgaaaaagtggcaccgagtcggtgctttttt

SEQ ID NO: 63 - DNA, 102 bp, synthetic construct
  misc_feature 1..102: note = Description of sequence: polypeptide, name: BBa_B0034 sgRNA (non-coding)
  source 1..102: mol_type = other DNA
tggtacctttctcctctttagttttagagctagaaatagcaagttaaaataaggctagtccgttatcaacttgaaaaagtggcaccgagtcggtgctttttt

SEQ ID NO: 64 - DNA, 50 bp, synthetic construct
  misc_feature 1..50: note = Description of sequence: polypeptide, name: PBD(10)rep top
  source 1..50: mol_type = other DNA
ttgcaaattaatcatccggctattataattaggactgagctagcttgcaa

SEQ ID NO: 65 - DNA, 50 bp, synthetic construct
  misc_feature 1..50: note = Description of sequence: polypeptide, name: PBD(10)rep bottom
  source 1..50: mol_type = other DNA
aacgtttaattagtaggccgataatattaatcctgactcgatcgaacgtt

SEQ ID NO: 66 - DNA, 41 bp, synthetic construct
  misc_feature 1..41: note = Description of sequence: polypeptide, name: PBD(17)rep top
  source 1..41: mol_type = other DNA
attatacctaggttgcaattaaattgcaaggctcgtataat

SEQ ID NO: 67 - DNA, 41 bp, synthetic construct
  misc_feature 1..41: note = Description of sequence: polypeptide, name: PBD(17)rep bottom
  source 1..41: mol_type = other DNA
taatatggatccaacgttaatttaacgttccgagcatatta

SEQ ID NO: 68 - DNA, 52 bp, synthetic construct
  misc_feature 1..52: note = Description of sequence: polypeptide, name: PBD(35)rep bottom
  source 1..52: mol_type = other DNA
taatatggatcctgactcgatcgaacgtttaattagtaggccgagcatatta